(19) United States
(12) Patent Application Publication
(10) Pub. No.: US 2011/0046185 A1
(43) Pub. Date: Feb. 24, 2011

(54) Arylchalcogenoarylalkyl-substituted imidazolidine-2,4-diones, process for preparation thereof, medicaments comprising these compounds and use thereof

(75) Inventors: Gerhard JAEHNE, Frankfurt (DE); Peter BELOW, Frankfurt (DE); Siegfried STENGELIN, Eppstein-Bremthal (DE); Matthias GOSSEL, Hofheim (DE); Thomas KLABUNDE, Frankfurt (DE); Irvin WINKLER, Liederbach (DE)

Correspondence Address: Scully, Scott, Murphy & Presser, P.C., 400 Garden City Plaza, Suite 300, Garden City, NY, 11530, US

(73) Assignee: SANOFI-AVENTIS, Paris (FR)

(21) Appl. No.: 12/852,188

(22) Filed: Aug. 6, 2010

Related U.S. Application Data
(63) Continuation of application No. PCT/EP2009/000591, filed on Jan. 30, 2009.

(30) Foreign Application Priority Data
Feb. 7, 2008 (EP) 08290131.5

(51) Int. Cl. A61K 31/4439; C07D 233/72; A61K 31/4166; A61P 3/10; A61P 3/04; A61P 25/32; A61P 25/34; A61P 25/18; A61P 25/28; C07D 401/04; C07D 409/04; C07D 417/10; A61K 31/428

(52) U.S. Cl. 514/341; 548/321.1; 514/391; 548/316.1; 546/274.4; 548/315.1; 548/180; 514/367

(57) ABSTRACT

The invention relates to compounds of formula (I) wherein the groups R and R′ A, D, E, G, L, p and R1 to R10 have the stated meanings and to their physiologically compatible salts. Said compounds are suitable, for example, as anti-obesity drugs.

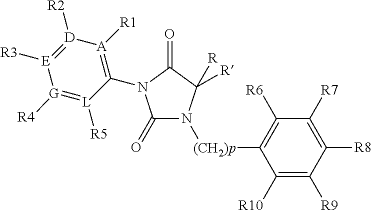

The invention relates to imidazolidine-2,4-diones which are substituted by an aralkyl radical and to the physiologically compatible salts thereof.

Structurally similar imidazoline-2,4-diones have already been described in U.S. Pat. No. 5,411,981. WO2004/031160A2 discloses structurally similar compounds, but these always bear a thiocarbonyl group on the imidazolidine ring. The most structurally similar examples 42, 90 and 116 from WO2004/031160A were, like the inventive compounds, tested in the hCB1R FLIPR assay and exhibited an IC50 value of >10 μm and therefore have to be considered as inactive.

It was an object of the invention to provide compounds which display a therapeutically utilizable action. In particular, it was an object of the invention to find novel compounds which are suitable for the treatment of metabolic syndrome, of type II diabetes and of obesity.

The invention therefore relates to compounds of the formula I

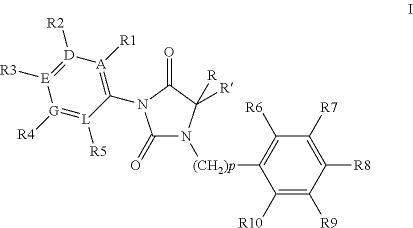

in which
R, R′ are each independently H, (CH₂)ₙ-aryl, (C₁-C₆)-alkyl, where (C₁-C₆)-alkyl or the aryl radical may be substituted by halogen, O-R14, S(O)ₘ-R12 or NR13R15;
or R and R′ together form a ring having from three to eight carbon atoms, where one carbon atom may be replaced by O, S(O)ₘ, N—(CH₂)ₙ—CO—NH-aryl, NR13 or NR15;
m is 0, 1, 2;
n is 0, 1, 2, 3, 4;
p is 1, 2, 3, 4, 5;
q is 1, 2, 3, 4;
r is 2, 3, 4, 5, 6;
v is 0, 1, 2, 3, 4;
A, D, E, G, L are each independently C or N, where there is no corresponding R1, R2, R3, R4, R5 substituent when they are defined as N, or R2-D=E-R3 or R4-G=L-R5 is defined as S or O;
R1, R2, R3, R4, R5 are each independently H, F, Cl, Br, I, CN, N₃, NC, NO₂, CF₃, (C₁-C₈)-alkyl, (C₃-C₈)-cycloalkyl, (CH₂)_{q}-[(C₃-C₈)-cycloalkyl], (CH₂)ₙ—[(C₃-C₈)-cycloalkenyl], (CH₂)ₙ-[(C₇-C₁₂)-bicycloalkyl], (CH₂)ₙ-[(C₇-C₁₂)-bicycloalkenyl], (CH₂)ₙ—[(C₇-C₁₂)-tricycloalkyl], adamantan-1-yl, adamantan-2-yl, (CH₂)ₙ-aryl, (CH₂)ₙ-heteroaryl, OCF₃, O-R11, NR13R15, NH—CN, S(O)ₘ-R12, SO₂—NH₂, SO₂—N═CH—N(CH₃)₂, SO₂—NH—CO-R12, SO₂—NH—CO—NHR12, SO₂—NH—CO-R16, SO₂—NH—[(C₁-C₈)-alkyl], SO₂—NH—[(C₃-C₈)-cycloalkyl], SO₂—NH—(CH₂)ᵣ—OH, SO₂—NH—(CH₂)ₙ-aryl, SO₂—NH—(CH₂)ₙ-heteroaryl, SO₂—N[(C₁-C₈)-alkyl]₂, SO₂—N[(CH₂)ₙ-aryl][(CH₂)ₙ-heteroaryl], SO₂-R16, SF₅, CO—O[(C₁-C₈)-alkyl], CO—O[(C₃-C₈)-cycloalkyl], CO—O—(CH₂)ᵣ—NH₂, CO—O—(CH₂)ₙ-aryl, CO—O—(CH₂)ₙ-heteroaryl, CO—NH₂, CO—NH—CN, CO—NH—[(C₁-C₈)-alkyl], CO—NH—(CH₂)ᵣ—OH, CO—N[(C₁-C₈)-alkyl]₂, CO—NH—[(C₃-C₈)-cycloalkyl], CO—N[(C₃-C₈)-cycloalkyl]₂, C(═NH)—O—[(C₁-C₆-alkyl)], C(═NH)—NH₂, C(═NH)—NR12R13, C(═NH)—R16, C(═NR13)-NR12R13, (CH₂)ₙ—C(═NSO₂-R12)NH₂, CO—NH—SO₂-R16, CO—NH—SO₂—NHR12, CO-R16, COOH, CO—(C₁-C₈)-alkyl, CO—(C₃-C₈)-cycloalkyl, CO—(CH₂)ₙ-[(C₇-C₁₂)-bicycloalkyl], CO—(CH₂)ₙ-[(C₇-C₁₂)-tricycloalkyl], CO-aryl, CO-heteroaryl, CH(OH)-aryl, CH(OH)-heteroaryl, CH[O—(C₁-C₆)-alkyl]-aryl, CH[O—(C₁-C₆)-alkyl]-heteroaryl, CHF-aryl, CHF-heteroaryl, CF₂-aryl, CF₂-heteroaryl, CHO, CH₂—OH, CH₂—CN, CH₂—O-R12, CH₂—O—(CH₂)ₙ—CO—O[(C₁-C₈)-alkyl], CH₂—O—(CH₂)ₙ—CO—NH₂, CH₂—O—(CH₂)_{q}—COOH,
   where the alkyl, cycloalkyl, cycloalkenyl, bicycloalkyl, bicycloalkenyl and tricycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, (CH₂)ₙ-aryl, O—(CH₂)ₙ-aryl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms; and
   where the R1 and R2, R2 and R3, R3 and R4 or R4 and F5 radicals may in each case together be defined as —(CH₂)₃— or —(CH₂)₄— or —CH═CH—CH═CH—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or the X-bicyclic heterocycle may be unsubstituted or mono- or polysubstituted by
   R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH-R13, (CH₂)ₙ—N(R13)₂, (CH₂)ₙ—NH—CN, (CH₂)ₙ—NH—SO₂-R16, (CH₂)ₙ—NH—(CH₂)ₙ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ-R12, SO₂-R16, SO₂—N═CH—N(CH₃)₂,

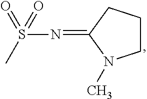

SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—-O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂—R16, SO₂—N═CH—N(CH₃)₂,

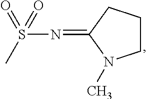

(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
   where at least one of the R6, R7, R8, R9 and R10 radicals is always defined as X-bicyclic heterocycle, X-aryl or X-heteroaryl and
   where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ
R11 is H, (C₁-C₈)-alkyl, (C₂-C₁₀)-alkenyl, (C₂-C₁₀)-alkynyl, (C₃-C₈)-cycloalkyl, (CH₂)_{q}-[(C₃-C₈)-cycloalkyl], (CH₂)ₙ-[(C₇-C₁₂)-bicycloalkyl], (CH₂)ₙ-[(C₃-C₁₀)-cycloalkenyl], (CH₂)ₙ-[(C₃-C₁₀)-bicycloalkenyl], (CH₂)ₙ-[(C₇-C₁₂)-tricycloalkyl], (CH₂)ₙ-aryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)_{q}—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (CH₂)ₙ—CO—N[(C₃-C₈)-cycloalkyl]₂, (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CR21[(CO—O(C₁-C₆)-alkyl)]₂, (CH₂)ₙ—CR21 (CONH₂)₂, (CH₂)ₙ—CR21 (COOH)₂, (CH₂)ₙ—CR21R22CO—O[(C₁-C₆)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₈)-alkyl], (CH₂)ₙ—C(CH₃)₂—CO—O[(C₃-C₈)-cycloalkyl], (CH₂)ₙ—C(CH₃)₂—CO—O—(CH₂)ᵣ—NH₂, (CH₂)ₙ—C(CH₃)₂—CO—O—(CH₂)ₙ-aryl, (CH₂)ₙ—C(CH₃)₂—CO—O—(CH₂)ₙ-heteroaryl, (CH₂)ₙ—C(CH₃)₂—CO—NH₂, (CH₂)ₙ—C(CH₃)₂—CO—NH—[(C₁-C₈)-alkyl], (CH₂)ₙ—C(CH₃)₂—CO—NH—(CH₂)ᵣ—OH, (CH₂)ₙ—C(CH₃)₂—CO—N[(C₁-C₈)-alkyl]₂, (CH₂)ₙ—C(CH₃)₂—CO—NH—[(C₃-C₈)-cycloalkyl], (CH₂)ₙ—C(CH₃)₂—CO—N[(C₃-C₈)-cycloalkyl]₂, (CH₂)ₙ—C(CH₃)₂—COOH, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl, bicycloalkyl, cycloalkenyl and bicycloalkenyl radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R12 is H, (C₁-C₈)-alkyl, (C₃-C₈)-cycloalkyl, (CH₂)_{q}-[(C₃-C₈)-cycloalkyl], (CH₂)ₙ-[(C₇-C₁₂)-bicycloalkyl], (CH₂)ₙ—[(C₇-C₁₂)-tricycloalkyl], (CH₂)ₙ-aryl, (CH₂)ₙ-heteroaryl, where the alkyl or cycloalkyl radicals may be substituted by fluorine atoms,
   and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R13 is H, SO₂—[(C₁-C₈)-alkyl], SO₂—[(C₃-C₈)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂, where the alkyl and cycloalkyl radicals may be substituted by fluorine atoms and where the aryl or heteroaryl radical may be substituted by halogen, CN, CF₃, (C₁-C₆)-alkyl, (C₃-C₆)-cycloalkyl, O—[(C₁-C₆)-alkyl], S(O)ₘ-[(C₁-C₆)-alkyl], SO₂—NH₂, COOH, CONH₂, CO—[O(C₁-C₆)-alkyl], CO—(C₁-C₆)-alkyl and where the alkyl radicals may be substituted by fluorine atoms;
R14 is H, (C₁-C₈)-alkyl, (C₃-C₈)-cycloalkyl, (CH₂)_{q}-[(C₃-C₈)-cycloalkyl], (CH₂)ₙ-aryl, (CH₂)ₙ-heteroaryl, (CH₂)ₙ—CO—[O—(C₁-C₈)-alkyl], (CH₂)ₙ—CO—[O—(C₃-C₈)-cycloalkyl], (CH₂)ₙ—CO—[O—(CH₂)ₙ-aryl], (CH₂)ₙ—CO—[O—(CH₂)ₙ-heteroaryl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (CH₂)ₙ—CO—N[(C₃-C₈)-cycloalkyl]₂, (CH₂)ₙ—C(CH₃)₂—CO—O[(C₁-C₈)]-alkyl, (CH₂)ₙ—C(CH₃)₂—CO—O[(C₃-C₈)]-cycloalkyl, (CH₂)ₙ—C(CH₃)₂—CO—O—(CH₂)ₙ—NH₂, (CH₂)ₙ—C(CH₃)₂—CO—NH₂, (CH₂)ₙ—C(CH₃)₂—CO—NH—(CH₂)ᵣ—OH, (CH₂)ₙ—C(CH₃)₂—COOH, where the alkyl and cycloalkyl radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R15 is H, (C₁-C₈)-alkyl, (C₃-C₈)-cycloalkyl, (CH₂)ₙ-aryl, (CH₂)ₙ-heteroaryl, (CH₂)ₙ—CO—[O—(C₁-C₈)-alkyl], (CH₂)ₙ—CO—[O—(C₃-C₈)-cycloalkyl], (CH₂)ₙ—CO—[O—(CH₂)ₙ-aryl], (CH₂)ₙ—CO—[O—(CH₂)ₙ-heteroaryl], CO—[(C₁-C₈)-alkyl], CO—[(C₃-C₈)-cycloalkyl], CO-aryl, CO-heteroaryl, (CH₂)ₙ—CO—NH₂, (CH₂)_{q}—COOH, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (CH₂)ₙ—C(CH₃)₂—CO—NH₂, (CH₂)ₙ—C(CH₃)₂—COOH, where the alkyl and cycloalkyl radicals may be substituted by fluorine atoms and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, 2-cyanopyrrolidin-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholin-4-yl, thiomorpholine-1,1-dioxid-4-yl, NH—(CH₂)ₙ-aryl-(CH₂)ₙ-aryl, NH—(CH₂)ᵣ—OH, NH—CH(CH₂OH)₂, NH—C(CH₂OH)₃, N[(C₁-C₆)-alkyl-OH]₂, N[(C₁-C₆)-alkyl][(C₁-C₆)-alkyl-OH], D-glucamin-N-yl, N-methyl-D-glucamin-N-yl, NH—[(C₁-C₈)-alkyl]-CO—O(C₁-C₆)-alkyl, NH—[(C₁-C₈)-alkyl]-COOH, NH—[(C₁-C₈)-alkyl]-CONH₂, N[(C₁-C₆)-alkyl][(C₁-C₈)-alkyl]-CO—O(C₁-C₆)-alkyl, N[(C₁-C₆)-alkyl][(C₁-C₈)-alkyl]-COOH, N[(C₁-C₆)-alkyl][(C₁-C₈)-alkyl]-CONH₂, NH—[C(H)(aryl)]-CO—O(C₁-C₆)-alkyl, NH—[C(H)(aryl)]-COOH, NH—[C(H)(aryl)]-CONH₂, N[(C₁-C₆)-alkyl][C(H)(aryl)]-CO—O(C₁-C₆)-alkyl, N[(C₁-C₆)-alkyl][C(H)(aryl)]-COOH, N[(C₁-C₆)-alkyl][C(H)(aryl)]-CONH₂, NH—[C(H)(heteroaryl)]-CO—O(C₁-C₆)-alkyl, NH—[C(H)(heteroaryl)]-COOH, NH—[C(H)(heteroaryl)]-CONH₂, N[(C₁-C₆)-alkyl][C(H)(heteroaryl)]-CO—O(C₁-C₆)-alkyl, N[(C₁-C₆)-alkyl][C(H)(heteroaryl)]-COOH, N[(C₁-C₆)-alkyl][C(H)(heteroaryl)]-CONH₂, N[(C₁-C₆)-alkyl][(C₃-C₈)-cycloalkyl]-CO—O(C₁-C₆)-alkyl, N[(C₁-C₆)-alkyl][(C₃-C₈)-cycloalkyl]-COOH, N[(C₁-C₆)-alkyl][(C₃-C₈)-cycloalkyl]-CONH₂, NH—[(C₃-C₈)-cycloalkyl]-CO—O(C₁-C₆)-alkyl, NH—[(C₃-C₈)-cycloalkyl]-COOH, NH—[(C₃-C₈)-cycloalkyl]-CONH₂, NH—(CH₂)ᵣ—SO2-(C₁-C₆)-alkyl, NH—[(C₁-C₆)-alkyl]-SO₃H, NH—[(C₁-C₆)-alkyl]-SO₂—NH₂, N[(C₁-C₆)-alkyl]{[(C₁-C₆)-alkyl]-SO₃H},
   where the alcohol (OH) or ketone (C═O) functions may be replaced by F or CF₂;
R18 is (C₁-C₈)-alkyl, (C₃-C₈)-cycloalkyl, (CH₂)_{q}-[(C₃-C₈)-cycloalkyl], (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₆)-alkyl, O—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
R20 is H, (C₁-C₆)-alkyl, (C₃-C₈)-cycloalkyl, aryl, [(C₁-C₆)-alkyl]-aryl;
R21 is H, F, CF₃, (C₁-C₆)-alkyl, (C₃-C₈)-cycloalkyl, OH, O—(C₁-C₆)-alkyl, O—(C₃-C₈)-cycloalkyl, O—(CH₂)ₙ-aryl, O—(CO)—(C₁-C₆)-alkyl, O—(CO)—(C₃-C₈)-cycloalkyl, O—(CO)—O—(C₁-C₆)-alkyl, O—(CO)—O—(C₃-C₈)-cycloalkyl, NH—[(C₁-C₆)-alkyl]-aryl, NH₂, NH—(C₁-C₆)-alkyl, NH—(CO)—(C₁-C₆)-alkyl;
R22 is H, CF₃, (C₁-C₆)-alkyl, aryl, [(C₁-C₆)-alkyl]-aryl;
   and physiologically compatible salts thereof.

Preference is given to compounds of the formula I in which one or more radicals are each defined as follows:
R, R′ are each independently H, (CH₂)ₙ-aryl, (C₁-C₆)-alkyl, where (C₁-C₆)-alkyl or the aryl radical may be substituted by halogen,
or R and R′ together form a ring having from three to eight carbon atoms, where one carbon atom may be replaced by O, S(O)ₘ, NR13 or NR15;
m is 0, 1, 2;
n is 0, 1, 2, 3, 4;
p is 1, 2, 3;
q is 1, 2, 3;
r is 2, 3, 4;
v is 0, 1, 2, 3;
A, D, E, G, L are each independently C or N, where there is no corresponding R1, R2, R3, R4, R5 substituent when they are defined as N, or R2-D=E-R3 or R4-G=L-R5 are defined as S or O;
R1, R2, R3, R4, R5 are each independently H, F, Cl, Br, I, CN, CF₃, (C₁-C₆)-alkyl, (C₃-C₆)-cycloalkyl, (CH₂)_{q}-[(C₃-C₆)-cycloalkyl], (CH₂)ₙ-[(C₇-C₁₀)-bicycloalkyl], (CH₂)ₙ—[(C₇-C₁₂)-tricycloalkyl], adamantan-1-yl, adamantan-2-yl, (CH₂)ₙ-aryl, (CH₂)ₙ-heteroaryl, OCF₃, O-R11, NR13R15, NH—CN, S(O)ₘ-R12, SO₂—NH₂, SO₂—N═CH—N(CH₃)₂, SO₂—NH—CO-R12, SO₂—NH—CO—NHR12, SO₂—NH—CO—R16, SO₂—NH—[(C₁-C₆)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₆)-alkyl]₂, SO₂-R16, SF₅, CO—O[(C₁-C₆)-alkyl], CO—O[(C₃-C₆)-cycloalkyl], CO—O—(CH₂)ₙ-aryl, CO—O—(CH₂)ₙ-heteroaryl, CO—NH₂, CO—NH—CN, CO—NH—[(C₁-C₆)-alkyl], CO—N[(C₁-C₆)-alkyl]₂, CO—NH—[(C₃-C₆)-cycloalkyl], C(═NH)—O—[(C₁-C₆-alkyl)], C(═NH)—NH₂, C(═NH)—NR12R13, C(═NH)—R16, C(═NR13)-NR12R13, (CH₂)ₙ—C(═NSO₂-R12)NH₂, CO—NH—SO₂-R16, CO—NH—SO₂—NHR12, CO-R16, COOH, CO—(C₁-C₆)-alkyl, CO—(C₃-C₆)-cycloalkyl, CO-aryl, CO-heteroaryl, CH(OH)-aryl, CH(OH)-heteroaryl, CH[O—(C₁-C₄)-alkyl]-aryl, CH[O—(C₁-C₄)-alkyl]-heteroaryl, CHF-aryl, CHF-heteroaryl, CF₂-aryl, CF₂-heteroaryl, CHO, CH₂—OH, CH₂—CN, CH₂—O-R12, CH₂—O—(CH₂)_{q}—COOH,
   where the alkyl, cycloalkyl, cycloalkenyl, bicycloalkyl, bicycloalkenyl and tricycloalkyl radicals may be substituted by fluorine atoms and where the aryl or heteroaryl radicals may be substituted by halogen, CN, (C₁-C₄)-alkyl, (C₃-C₆)-cycloalkyl, O—(C₁-C₄)-alkyl, (CH₂)ₙ-aryl, O—(CH₂)ₙ-aryl, S(O)ₘ—(C₁-C₄)-alkyl, SO₂—NH₂, COOH, CONH₂, CO—O(C₁-C₄)-alkyl CO—(C₁-C₄)-alkyl and where the alkyl radicals may be substituted by fluorine atoms; and
   where the R1 and R2, R2 and R3, R3 and R4 or R4 and R5 radicals may in each case together be defined as —(CH₂)₃— or —(CH₂)₄— or —CH═CH—CH═CH—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or the X-bicyclic heterocycle may be unsubstituted or mono- or polysubstituted by
   R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ₙ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ-R12, SO₂-R16, SO₂—N═CH—N(CH₃)₂,

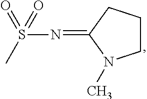

SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)—alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—_—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂,

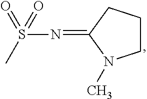

(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
   where at least one of the R6, R7, R8, R9 and R10 radicals is always defined as X-bicyclic heterocycle, X-aryl or X-heteroaryl and
   where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ,
R11 is H, (C₁-C₈)-alkyl, (C₂-C₆)-alkenyl, (C₂-C₆)-alkynyl, (C₃-C₆)-cycloalkyl, (CH₂)_{q}—[(C₃-C₆)-cycloalkyl], (CH₂)ₙ-[(C₇-C₁₂)-bicycloalkyl], (CH₂)ₙ—[(C₇-C₁₂)-tricycloalkyl], (CH₂)ₙ-aryl, (CH₂)ₙ—CO—[O—(C₁-C₆)-alkyl], (CH₂)ₙ—CO—[O—(C₃-C₆)-cycloalkyl], (CH₂)ₙ—CO—[(C₁-C₆)-alkyl], (CH₂)ₙ—CO—[(C₃-C₆)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)_{q}—CO—NH—CN, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₆)-alkyl], (CH₂)ₙ—CO—N[(C₁-C₆)-alkyl]₂, (CH₂)ₙ—CO—NH—[(C₃-C₆)-cycloalkyl], (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkenyl-COOH, (CH₂)ₙ—CR21[(CO—O(C₁-C₄)-alkyl)]₂, (CH₂)ₙ—CR21 (CONH₂)₂, (CH₂)ₙ—CR21(COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₈)-alkyl], (CH₂)ₙ—C(CH₃)₂—CO—O[(C₃-C₈)-cycloalkyl], (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₆)-alkyl], (CH₂)ₙ—C(CH₃)₂—CO—N[(C₁-C₆)-alkyl]₂, (CH₂)ₙ—C(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₆)-alkyl], (CH₂)ₙ—CO—NH—C(CH₃)₂—CONH₂, (CH₂)ₙ—CO—NH—C(CH₃)₂—COOH,
   where the alkyl, alkenyl, alkynyl and cycloalkyl and bicycloalkyl radicals may be substituted by fluorine atoms
   and where the aryl or heteroaryl radical may be substituted by halogen, CN, (C₁-C₄)-alkyl, (C₃-C₆)-cycloalkyl, O—(C₁-C₄)-alkyl, S(O)ₘ—(C₁-C₄)-alkyl, SO₂—NH₂, COOH, CONH₂, CO—O(C₁-C₆)-alkyl, CO—(C₁-C₆)-alkyl, and
   where the alkyl radicals may be substituted by fluorine atoms;
R12 is H, (C₁-C₆)-alkyl, (C₃-C₆)-cycloalkyl, (CH₂)_{q}-[(C₃-C₆)-cycloalkyl], (CH₂)ₙ-aryl, (CH₂)ₙ-heteroaryl,
   where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, CO—(C₁-C₄)-alkyl and where the alkyl radicals may be substituted by fluorine atoms;
R13 is H, SO₂—[(C₁-C₆)-alkyl], SO₂—[(C₃-C₆)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂, where the alkyl and cycloalkyl radicals may be substituted by fluorine atoms and where the aryl or heteroaryl radical may be substituted by halogen, CN, CF₃, (C₁-C₄)-alkyl, (C₃-C₆)-cycloalkyl, O—[(C₁-C₄)-alkyl], S(O)ₘ-[(C₁-C₄)-alkyl], SO₂—NH₂, COOH, CONH₂, CO—[O(C₁-C₄)-alkyl], CO—(C₁-C₄)-alkyl, and where the alkyl radicals may be substituted by fluorine atoms;
R15 is H, (C₁-C₈)-alkyl,
   where the alkyl radical may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholin-4-yl, thiomorpholine-1,1-dioxid-4-yl, NH—(CH₂)ₙ-aryl-(CH₂)ₙ-aryl, NH—(CH₂)ᵣ—OH, NH—CH(CH₂OH)₂, NH—C(CH₂OH)₃, N[(C₁-C₄)-alkyl-OH]₂, D-glucamin-N-yl, N-methyl-D-glucamin-N-yl, NH—[(C₁-C₆)-alkyl]-CO—O(C₁-C₄)-alkyl, NH—[(C₁-C₄)-alkyl]-COOH, NH—[(C₁-C₄)-alkyl]-CONH₂, N[(C₁-C₆)-alkyl][(C₁-C₈)-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(CH₂)ᵣ—SO2-(C₁-C₄)-alkyl, NH—[(C₁-C₄)-alkyl]-SO₃H, NH—[(C₁-C₄)-alkyl]-SO₂—NH₂, where the alcohol (OH) or ketone (C═O) functions may be replaced by F or CF₂;
R18 is (C₁-C₆)-alkyl, (C₃-C₆)-cycloalkyl, (CH₂)ₙ-[(C₃-C₆)-cycloalkyl], (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₄)-alkyl, O—(C₁-C₄)-alkyl, SO₂—NH₂, COOH, CONH₂, CO—[O(C₁-C₄)-alkyl], CO—(C₁-C₄)-alkyl, and where the alkyl radicals may be substituted by fluorine atoms;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
   and the physiologically compatible salts thereof.

Particular preference is given to compounds of the formula I in which one or more radicals are each defined as follows:
R, R′ are each independently H, (CH₂)ₙ-aryl (C₁-C₄)-alkyl where (C₁-C₄)-alkyl or the aryl radical may be substituted by halogen
or R and R′ together form a ring having from three to eight carbon atoms where one carbon atom may be replaced by O, S(O)ₘ, NR13 or NR15;
m is 0, 1, 2;
n is 0, 1, 2;
p is 1, 2, 3;
q is 1, 2, 3;
r is 2, 3, 4;
v is 0, 1, 2;
A, D, E, G, L are each independently C or N, where there is no corresponding R1, R2, R3, R4, R5 substituents when they are defined as N, or R2-D=E-R3 or R4-G=L-R5 are defined as S or O;
R1, R2, R3, R4, R5 are each independently H, F, Cl, Br, I, CN, CF₃, (C₁-C₄)-alkyl, (C₃-C₆)-cycloalkyl, adamantan-1-yl, adamantan-2-yl, (CH₂)ₙ-aryl, (CH₂)ₙ-heteroaryl, OCF₃, O-R11, NR13R15, S(O)ₘ-R12, SO₂—NH₂, SO₂—N═CH—N(CH₃)₂, SO₂—NH—CO-R12, SO₂—NH—CO—NHR12, SO₂—NH—CO-R16, SO₂—NH—[(C₁-C₄)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₄)-alkyl]₂, SO₂-R16, SF₅, CO—O[(C₁-C₄)-alkyl], CO—O[(C₃-C₄)-cycloalkyl], CO—NH₂, CO—NH—[(C₁-C₄)-alkyl], CO—N[(C₁-C₄)-alkyl]₂, CO—NH—[(C₃-C₆)-cycloalkyl], C(═NH)—O—[(C₁-C₄-alkyl)], C(═NH)—NH₂, C(═NH)—NR12R13, C(═NH)—R16, C(═NR13)-NR12R13, (CH₂)ₙ—C(═NSO₂-R12)NH₂, CO—NH—SO₂-R16, CO—NH—SO₂—NHR12, CO-R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-Aryl, CF₂-heteroaryl, CH₂—OH, CH₂—CN, CH₂—O-R12, CH₂—O—(CH₂)_{q}—COOH,
   where the alkyl and cycloalkyl radicals may be substituted by fluorine atoms and where the aryl or heteroaryl radicals may be substituted by halogen, CN, (C₁-C₄)-alkyl, (C₃-C₆)-cycloalkyl, O—(C₁-C₄)-alkyl, (CH₂)ₙ-aryl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl, and where the alkyl radicals may be substituted by fluorine atoms; and
   where the R1 and R2, R2 and R3, R3 and R4 or R4 and R5 radicals may in each case together be defined as —(CH₂)₃— or —(CH₂)₄— or —CH═CH—CH═CH—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or the X-bicyclic heterocycle may be unsubstituted or mono- or polysubstituted by
   R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ₙ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ₙ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ-R12, SO₂-R16, SO₂—N═CH—N(CH₃)₂,

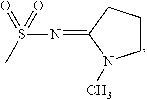

SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—N[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂—R16, SO₂—N═CH—N(CH₃)₂,

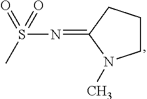

(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
   where at least one of the R6, R7, R8, R9 and R10 radicals is always defined as X-bicyclic heterocycle, X-aryl or X-heteroaryl and
   where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ
R11 is H, (C₁-C₈)-alkyl, (C₂-C₆)-alkynyl, (C₃-C₆)-cycloalkyl, (CH₂)ₙ-aryl, (CH₂)ₙ—CO—[O—(C₁-C₄)-alkyl], (CH₂)ₙ—CO—[O—(C₃-C₆)-cycloalkyl], (CH₂)ₙ—CO—[(C₁-C₄)-alkyl], (CH₂)ₙ—CO—[(C₃-C₆)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₄)]-alkyl, (CH₂)ₙ—C(CH₃)₂—CO—O[(C₃-C₆)]-cycloalkyl, (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₄)-alkyl], (CH₂)ₙ—C(CH₃)₂—CO—N[(C₁-C₄)-alkyl]₂, (CH₂)ₙ—(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₄)-alkyl], (CH₂)ₙ—CO—NH—C(CH₃)₂—CONH₂, (CH₂)ₙ—CO—NH—C(CH₃)₂—COOH,
   where the alkyl, alkenyl, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₆)-alkyl, and where the alkyl radicals may be substituted by fluorine atoms;
R12 is H, (C₁-C₃)-alkyl, (C₃-C₆)-cycloalkyl, (CH₂)_{q}-[(C₃-C₆)-cycloalkyl], (CH₂)ₙ-aryl, (CH₂)ₙ-heteroaryl,
   where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂,
   where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ-[(C₁-C₄)-alkyl], SO₂—NH₂, COOH, CONH₂, CO—[O(C₁-C₄)-alkyl], and where the alkyl radicals may be substituted by fluorine atoms;
R15 is H, (C₁-C₈)-alkyl,
   where the alkyl radical may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholine-1,1-dioxid-4-yl, NH—(CH₂)ᵣ—OH, NH—CH(CH₂OH)₂, NH—C(CH₂OH)₃, N[(C₁-C₄)-alkyl-OH]₂, NH—[(C₁-C₄)-alkyl]-COOH, NH—[(C₁-C₄)-alkyl]-CONH₂, N[(C₁-C₆)-alkyl][C₁-C₈-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(CH₂)ₙ—SO₂—(C₁-C₄)-alkyl, NH—[(C₁-C₄)-alkyl]-SO₃H, NH—[(C₁-C₄)-alkyl]-SO₂—NH₂, where the alcohol (OH) or ketone (C═O) functions may be replaced by F or CF₂;
R18 is (C₁-C₄)-alkyl, (C₃-C₆)-cycloalkyl, (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₄)-alkyl, O—(C₁-C₄)-alkyl, SO₂—NH₂, COOH, CONH₂, CO—[O(C₁-C₄)-alkyl], and where the alkyl radicals may be substituted by fluorine atoms;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
   and the physiologically compatible salts thereof.

Very particular preference is given to compounds of the formula I in which one or more radicals are each defined as follows:
R, R′ are each independently H, aryl, (C₁-C₄)-alkyl, where (C₁-C₄)-alkyl or the aryl radical may be substituted by halogen;
or R and R′ together form a ring having from three to eight carbon atoms, where one carbon atom may be replaced by O, S(O)ₘ, NR13 or NR15;
m is 0, 1, 2;
n is 0, 1, 2;
p is 1, 2, 3;
q is 1, 2, 3;
r is 2, 3;
v is 0, 1, 2;
A, D, E, G, L are each independently C or N, where there is no corresponding R1, R2, R3, R4, R5 substituent when they are defined as N or R2-D=E-R3 or R4-G=L-R5 are defined as S or O;
R1, R2, R3, R4, R5 are each independently H, F, Cl, Br, I, CN, CF₃, (C₁-C₄)-alkyl, (C₃-C₆)-cycloalkyl, (CH₂)ₙ-aryl, (CH₂)ₙ-heteroaryl, OCF₃, O-R11, NR13R15, S(O)ₘ-R12, SO₂—NH₂, SO₂—NH—CO-R12, SO₂—NH—CO—NHR12, SO₂—NH—CO-R16, SO₂—NH—[(C₁-C₄)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₄)-alkyl]₂, SO₂-R16, SF₅, CO—O[(C₁-C₄)-alkyl], CO—O[(C₃-C₄)-cycloalkyl], CO—NH₂, CO—NH—[(C₁-C₄)-alkyl], CO—N[(C₁-C₄)-alkyl]₂, C(═NH)—NH₂, C(═NH)—NR12R13, C(═NH)—R16, (CH₂)ₙ—C(═NSO₂-R12)NH₂, CO—NH—SO₂-R16, CO—NH—SO₂—NHR12, CO-R16, COOH, CO—(C₁-C₄)-alkyl, CO—(C₃-C₆)-cycloalkyl, CO-Aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O-R12, CH₂—O—(CH₂)_{q}—COOH,
   where the alkyl or cycloalkyl radicals may be substituted by fluorine atoms and where the aryl or heteroaryl radicals may be substituted by halogen, CN, (C₁-C₄)-alkyl, O—(C₁-C₄)-alkyl, (CH₂)ₙ-aryl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl, and where the alkyl radicals may be substituted by fluorine atoms; and
   where the radicals R1 and R2, R2 and R3, R3 and R4 or R4 and R5 may be in each case together be defined as —(CH₂)₃— or —(CH₂)₄—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or X-bicyclic heterocycle may be unsubstituted or mono- or polysubstituted by
   R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ₙ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH₂]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ₙ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ-R12, SO₂-R16, SO₂—N═CH—N(CH₃)₂,

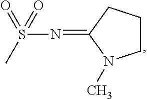

SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—-[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—N[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂,

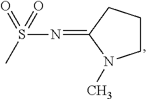

(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
   where at least one of the R6, R7, R8, R9 and R10 radicals is always defined as X-bicyclic heterocycle, X-aryl or X-heteroaryl and
   where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ,
R11 is H, (C₁-C₈)-alkyl, (C₂-C₆)-alkynyl, (C₃-C₆)-cycloalkyl, (CH₂)ₙ-aryl, (CH₂)ₙ—CO—[O—(C₁-C₄)-alkyl], (CH₂)ₙ—CO—[O—(C₃-C₆)-cycloalkyl], (CH₂)ₙ—CO—[(C₁-C₄)-alkyl], (CH₂)ₙ—CO—[(C₃-C₆)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₄)]-alkyl, (CH₂)ₙ—C(CH₃)₂—CO—O[(C₃-C₆)]-cycloalkyl, (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₄)-alkyl], (CH₂)ₙ—C(CH₃)₂—CO—N[(C₁-C₄)-alkyl]₂, (CH₂)ₙ—(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₄)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₆)-alkyl, and where the alkyl radicals may be substituted by fluorine atoms;
R12 is H, (C₁-C₄)-alkyl, (C₃-C₆)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂, where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ-[(C₁-C₄)-alkyl], SO₂—NH₂, COOH, CONH₂, CO—[O(C₁-C₄)-alkyl], and where the alkyl radicals may be substituted by fluorine atoms;
R15 is H, (C₁-C₈)-alkyl,
   where the alkyl radical may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholine-1,1-dioxid-4-yl, NH—(CH₂)ᵣ—OH, NH—CH(CH₂OH)₂, NH—C(CH₂OH)₃, N[(C₁-C₄)-alkyl-OH]₂, NH—[(C₁-C₄)-alkyl]-COOH, NH—[(C₁-C₄)-alkyl]-CONH₂, N[(C₁-C₆)-alkyl][C₁-C₈-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(CH₂)ᵣ—SO₂—(C₁-C₄)-alkyl, NH—[(C₁-C₄)-alkyl]-SO₃H, NH—[(C₁-C₄)-alkyl]-SO₂—NH₂, where the alcohol (OH) or ketone (C═O) functions may be replaced by F or CF₂;
R18 is (C₁-C₄)-alkyl, (C₃-C₆)-cycloalkyl, (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₄)-alkyl, O—(C₁-C₄)-alkyl, SO₂—NH₂, COOH, CONH₂, CO—[O(C₁-C₄)-alkyl], and where the alkyl radicals may be substituted by fluorine atoms;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
   and the physiologically compatible salts thereof.

Preference is further given to compounds of the formula Ia

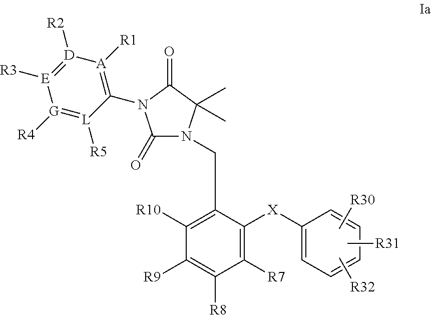

in which
m is 0, 1, 2;
n is 0, 1, 2;
q is 1, 2, 3;
r is 2, 3;
v is 0, 1, 2;
X is O, S(O)ₘ
A, D, E, G, L are each independently C or N, where there is no corresponding R1, R2, R3, R4, R5 substituent when they are defined as N, or R2-D=E-R3 or R4-G=L-R5 are defined as S or O;
R1, R2, R3, R4, R5 are each independently H, F, Cl, Br, I, CN, CF₃, (C₁-C₄)-alkyl, (C₃-C₆)-cycloalkyl, (CH₂)ₙ-aryl, (CH₂)ₙ-heteroaryl, OCF₃, O-R11, NR13R15, S(O)ₘ—R12, SO₂—NH₂, SO₂—NH—CO-R12, SO₂—NH—CO—NHR12, SO₂—NH—CO-R16, SO₂—NH—[(C₁-C₄)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₄)-alkyl]₂, SO₂-R16, SF₅, CO—O[(C₁-C₄)-alkyl], CO—O[(C₃-C₄)-cycloalkyl], CO—NH₂, CO—NH—[(C₁-C₄)-alkyl], CO—N[(C₁-C₄)-alkyl]₂, C(═NH)—NH₂, C(═NH)—NR12R13, C(═NH)—R16, (CH₂)ₙ—C(═NSO₂-R12)NH₂, CO—NH—SO₂-R16, CO—NH—SO₂—NHR12, CO-R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O-R12, CH₂—O—(CH₂)_{q}—COOH,
   where the alkyl and cycloalkyl radicals may be substituted by fluorine atoms and where the aryl or heteroaryl radicals may be substituted by halogen, CN, (C₁-C₄)-alkyl, O—(C₁-C₄)-alkyl, (CH₂)ₙ-aryl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl, and where the alkyl radicals may be substituted by fluorine atoms; and
   where the R1 and R2, R2 and R3, R3 and R4 or R4 and R5 radicals may each together be defined as —(CH₂)₃— or —(CH₂)₄—;
R7, R8, R9, R10 are each independently H, F, Cl, Br, I, CN, CF₃, (C₁-C₄)-alkyl, (C₂-C₄)-alkynyl, (C₃-C₆)-cycloalkyl, aryl, heteroaryl, (CH₂)ₙ—CO—[O—(C₁-C₄)-alkyl], (CH₂)ₙ—CO—[(C₁-C₄)-alkyl], (CH₂)ₙ—CO—NH₂, (CH₂)ₙ—COOH, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (CH₂)ₙ—CO-R16, (CH₂)ₙ—OH, (CH₂)ₙ—O—(C₁-C₄)-alkyl, (CH₂)ₙ—O—(C₃-C₆)-cycloalkyl, (CH₂)ₙ—O—(CH₂)ₙ—CO—[O—(C₁-C₄)-alkyl], (CH₂)ₙ—O—(CH₂)ₙ—CO—[(C₁-C₄)-alkyl], (CH₂)ₙ—O—(CH₂)_{q}—COOH, (CH₂)ₙ—O—(CH₂)ₙ—P(O)(OH)[O—(C₁-C₄)-alkyl], (CH₂)ₙ—O—(CH₂)ₙ—P(O)[O—(C₁-C₄)-alkyl]₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₄)-alkyl], (CH₂)ₙ—O—(CH₂)ₙ—CR21R22-CO—P[(C₁-C₄)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₄)-alkyl, (CH₂)ₙ—NH—(C₃-C₆)-cycloalkyl, (CH₂)ₙ—NH—(CH₂)ₙ—CO—[(C₁-C₄)-alkyl], (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₄)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₄)-alkyl], (CH₂)ₙ—NH—(CH₂)ₙ—SO₂—[(C₃-C₆)-cycloalkyl], (CH₂)ₙ—NH—SO₂—(CH₂)ₙ—NH—(C₁-C₄)-alkyl, (CH₂)ₙ—NH—SO₂—(CH₂)ₙ—NH—(C₃-C₆)-cycloalkyl, (CH₂)ₙ—NH—SO₂—(CH₂)ₙ—N[(C₁-C₄)-alkyl]₂, (CH₂)ₙ—NH—SO₂-R16, (CH₂)ₙ—NR12-CO—NH—(C₁-C₄)-alkyl, (CH₂)ₙ—NR12-CO—NH—(C₃-C₆)-cycloalkyl, (CH₂)ₙ—NR12-CO—NH₂, (CH₂)ₙ—NR12-CO—NH—SO₂—(C₁-C₄)-alkyl, (CH₂)ₙ—NH—CO—NH—(CH₂)ₙ—CO—[O—(C₁-C₄)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₄)-alkyl], (CH₂)ₙ—NH—C(═N—SO₂—(C₁-C₆)-alkyl)-NH₂, (CH₂)ₙ—NH—C(═N—SO₂—(C₁-C₄)-alkyl)-NH—[(C₁-C₄)-alkyl], (CH₂)ₙ—NH—C(═N—SO₂—NH₂)—NH₂, (CH₂)ₙ—NH—C(═N—SO₂—NH₂)—NH[(C₁-C₄)-alkyl], (CH₂)ₙ—NH—C(═NH)—N[(C₁-C₄)-alkyl]₂, (CH₂)ₙ—NH—C(═N—SO₂—(C₁-C₄)-alkyl)-N[(C₁-C₄)-alkyl]₂, (CH₂)ₙ—NH—(CH₂)ₙ—CO—NH—[(C₁-C₄)-alkyl], (CH₂)ₙ—NH—(CH₂)ₙ—CO—NH—(CH₂)ᵣ—OH, (CH₂)ₙ—S(O)ₘ—(C₁-C₄)-alkyl, (CH₂)ₙ—S(O)ₘ—(C₃-C₆)-cycloalkyl, SO₂—N═CH—N(CH₃)₂, (CH₂)ₙ—SO₂—NH—CO—(C₁-C₄)-alkyl, (CH₂)ₙ—SO₂—NH—CO—(C₃-C₆)-cycloalkyl, (CH₂)ₙ—SO₂—NH—(C₁-C₄)-alkyl, (CH₂)ₙ—SO₂—NH—(C₃-C₆)-cycloalkyl, SO₂—NH—(CH₂)ᵣ—OH, SO₂—NH—(CH₂)ᵣ—NH₂, SF₅, (CH₂)_{q}—CN, (CH₂)ₙ—CO—NH—SO₂—NHR12, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, where the alkyl and cycloalkyl radicals may be substituted by fluorine atoms, and where the aryl or heteroaryl radicals may be substituted by halogen, CN, (C₁-C₄)-alkyl, (C₃-C₆)-cycloalkyl, O—(C₁-C₄)-alkyl, S(O)ₘ—(C₁-C₄)-Alkyl, SO₂—NH₂, COOH, CONH₂, CO—[O(C₁-C₄)-alkyl], CO—(C₁-C₄)-alkyl, and where the alkyl radicals may be substituted by fluorine atoms;
R11 is H, (C₁-C₈)-alkyl, (C₂-C₆)-alkynyl, (C₃-C₆)-cycloalkyl, (CH₂)ₙ-aryl, (CH₂)ₙ—CO—[O—(C₁-C₄)-alkyl], (CH₂)ₙ—CO—[O—(C₃-C₆)-cycloalkyl], (CH₂)ₙ—CO—[(C₁-C₄)-alkyl], (CH₂)ₙ—CO—[(C₃-C₆)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₄)]-alkyl, (CH₂)ₙ—C(CH₃)₂—CO—O[(C₃-C₆)]-cycloalkyl, (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₄)-alkyl], (CH₂)ₙ—C(CH₃)₂—CO—N[(C₁-C₄)-alkyl]₂, (CH₂)ₙ—(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₄)-alkyl], (CH₂)ₙ—CO—NH—C(CH₃)₂—CONH₂, (CH₂)ₙ—CO—NH—C(CH₃)₂—COOH,
   where the alkyl, alkenyl, alkynyl and cycloalkyl radicals may be substituted by fluorine atoms, and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₆)-alkyl, and where the alkyl radicals may be substituted by fluorine atoms;
R30, R31, R32 are each independently R11, F, Cl, Br, I, CN, CF₃, (CH₂)ₙ—O-R11, O—R13, OCF₃, (CH₂)ₙ—NH—R11, (CH₂)ₙ—N[(CH₂)_{q}—CO—O(C₁-C₄)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—SO₂-R16, (CH₂)ₙ—NH—(CH₂)ₙ—SO₂—R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NH—(CH₂)ₙ—CO—NH—[(C₁-C₄)-alkyl], (CH₂)ₙ—NH—(CH₂)ₙ—CO—N[(C₁-C₄)-alkyl]₂, (CH₂)ₙ—NH—C(CH₃)₂—CO—O(C₁-C₄)-alkyl, (CH₂)ₙ—NH—C(CH₃)₂—CO—O(C₃-C₆)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₄)-alkyl], (CH₂)ₙ—NH—C(CH₃)₂—CO—N[(C₁-C₄)-alkyl]₂, (CH₂)ₙ—NH—C(CH₃)₂—COOH, S(O)ₘ-R12, SO₂-R16, SO₂—N═CH—N(CH₃)₂, SO₂—NH—CO-R12, SO₂—NHR12, SO₂—N[(C₁-C₄)-alkyl]₂, SF₅, COOH, CO—NH₂, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—C(═NH)—NHOH, (CH₂)ₙ—C(═NR13)NHR12, (CH₂)ₙ—C(═NR12)NR12R13, (CH₂)ₙ—C(═NSO₂-R12)NH₂,
   where the alkyl and cycloalkyl radicals may be substituted by fluorine atoms, and where the aryl or heteroaryl radicals may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₄)-alkyl, and where the alkyl radicals may be substituted by fluorine atoms,
      and where, when R3 is CN, NO₂ or halogen and R4 is CF₃ or halogen,
      R30, R31 or R32 is a substituent other than hydrogen;
      and the physiologically compatible salts thereof.

Preference is further given to compounds of the formula Ia

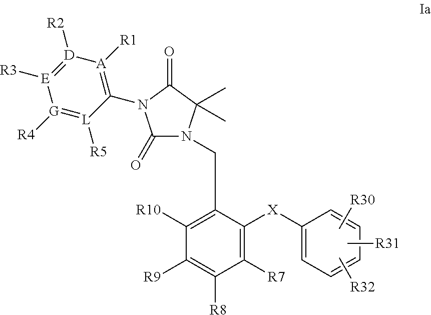

in which
m is 0, 1, 2;
n is 0, 1, 2;
X is O, S(O)ₘ
A, D, E, G, L are each independently C or N, where the corresponding R1, R2, R3, R4, R5 substituent is absent when they are defined as N, or R4-G=L-R5 is defined as S;
R1, R2, R3, R4, R5 are each independently H, F, Cl, Br, I, CN, CF₃, (C₁-C₄)-alkyl, (CH₂)ₙ-aryl, —O—(CH₂)ₙ-aryl, OCF₃, O—(C₁-C₈)-alkyl, S(O)ₘ—(C₁-C₈)-alkyl, CO—O[(C₁-C₄)-alkyl], CO—(C₁-C₄)-alkyl, CO-Aryl, CH₂—CN;
   where the alkyl radicals may be substituted by fluorine atoms;
R7, R8, R9, R10 are each H;
R30, R31, R32 are each independently H, (C₁-C₈)-alkyl, F, Cl, Br, —COOH, —COO(C₁-C₈)-alkyl, (CH₂)ₙ—CONH₂;
   and where, when R3 is CN, NO₂ or halogen and R4 is CF₃ or halogen, one of the R30, R31 or R32 radicals is a substituent other than hydrogen;
   and the physiologically compatible salts thereof.

In one embodiment, preference is given to compounds of the formula I in which p is 1.

In one embodiment, preference is given to compounds of the formula I in which R6 is O-aryl, where aryl may be substituted.

In one embodiment, preference is given to compounds of the formula I in which R6 is S(O)ₘ-aryl where m may be 0, 1 or 2 and where aryl may be substituted.

In one embodiment, preference is given to compounds of the formula I in which R7 is O-aryl, where aryl may be substituted.

In one embodiment, preference is given to compounds of the formula I in which R7 is S(O)ₘ-aryl where m may be 0, 1 or 2 and where aryl may be substituted.

In one embodiment, preference is given to compounds of the formula I in which R and R′ are each methyl.

In one embodiment, preference is given to compounds of the formula I in which A is CH.

In one embodiment, preference is given to compounds of the formula I in which A is N.

In one embodiment, preference is given to compounds of the formula I in which D is CH.

In one embodiment, preference is given to compounds of the formula I in which D is N.

In one embodiment, preference is given to compounds of the formula I in which E is CH.

In one embodiment, preference is given to compounds of the formula I in which E is N.

In one embodiment, preference is given to compounds of the formula I in which R4-G=L-R5 is defined as S.

In one embodiment, preference is given to compounds of the formula I in which one of the R1, R2, R3, R4 and R5 radicals is not H.

When radicals or substituents (for example R12) can occur more than once in the compounds of the formula I, they may all each independently be defined as specified and be the same or different.

The invention further provides both stereoisomer mixtures of the formula I and the pure stereoisomers of the formula I, and also diastereoisomer mixtures of the formula I and the pure diastereoisomers. The mixtures are separated, for example, by a chromatographic route.

The invention relates to compounds of the formula I in the form of their tautomers, racemates, racemic mixtures, stereoisomer mixtures, pure stereoisomers, diastereoisomer mixtures, pure diastereoisomers. The mixtures are separated, for example, by a chromatographic route.

The alkyl radicals in the substituents R1 to R18 and R and R′ may be either straight-chain or branched.

Owing to their high water solubility, pharmaceutically acceptable salts are particularly suitable for medical applications compared to the starting or base compounds. These salts must have a pharmaceutically acceptable anion or cation. Suitable pharmaceutically acceptable acid addition salts of the inventive compounds are salts of inorganic acids, such as hydrochloric acid, hydrobromic acid, phosphoric acid, metaphosphoric acid, nitric acid and sulfuric acid, and also organic acids, for example acetic acid, benzenesulfonic acid, benzoic acid, citric acid, ethanesulfonic acid, fumaric acid, gluconic acid, glycolic acid, isethionic acid, lactic acid, lactobionic acid, maleic acid, malic acid, methanesulfonic acid, succinic acid, p-toluenesulfonic acid and tartaric acid. Suitable pharmaceutically acceptable basic salts are ammonium salts, alkali metal salts (such as sodium and potassium salts), alkaline earth metal salts (such as magnesium and calcium salts), trometamol (2-amino-2-hydroxymethyl-1,3-propanediol), diethanolamine, lysine or ethylenediamine.

Salts with a pharmaceutically unacceptable anion, for example trifluoroacetate, are also included within the scope of the invention as useful intermediates for the preparation or purification of pharmaceutically acceptable salts and/or for use in non-therapeutic applications, for example in vitro applications.

The inventive compounds may also be present in different polymorphic forms, for example as amorphous and crystalline polymorphic forms. All polymorphic forms of the inventive compounds are included within the scope of the invention and are a further aspect of the invention.

Hereinafter, all references to “compound(s) of the formula I” relate to compound(s) of the formula I as described above, and to their salts and solvates as described herein.

An alkyl radical is understood to mean a straight-chain or branched hydrocarbon chain having from one to eight carbons, for example methyl, ethyl, isopropyl, tert-butyl, hexyl, heptyl, octyl. The alkyl radicals may be mono- or polysubstituted as described above.

A cycloalkyl radical is understood to mean a ring system which comprises one or more rings, is present in saturated or partially unsaturated form (with one or two double bonds) and is formed exclusively from carbon atoms, for example cyclopropyl, cyclopentyl, cyclopentenyl, cyclohexyl or adamantyl.

The cycloalkyl radicals may be mono- or polysubstituted by suitable groups as described above.

An aryl radical is understood to mean a phenyl, naphthyl, biphenyl, tetrahydronaphthyl, alpha- or beta-tetralonyl, indanyl or indan-1-onyl radical.

The aryl radicals may be mono- or polysubstituted by suitable groups as described above.

A heteroaryl radical is understood to mean aromatic rings and ring systems which, apart from carbon, also contain heteroatoms, for example nitrogen, oxygen or sulfur. This definition also includes ring systems in which the heteroaryl radical is fused to benzene rings. This likewise includes systems in which one or more CH group(s) has/have been replaced by CO═O or C═S, preferably C═O.

Suitable heteroaryl radicals are, for example, furyl, imidazolyl, benzimidazolyl, benzothiazolyl, indolyl, indolinyl, pyrimidinyl, pyridyl, pyrazinyl, pyrrolyl, thiazolyl, oxazolyl, isoxazolyl, thienyl, 1,2,3-triazolyl, 1,2,4-triazolyl, tetrazolyl, isoxazolyl, pyridazinyl, 1,3,5-triazinyl, 1,2,4-triazinyl; the 2H-pyridazin-3-one, dihydropyridazine-3,6-dione, imidazolidin-2-one, 1,3-dihydroimidazol-2-one, imidazolidin-2,5-dione, quinoline, isoquinoline, quinoxaline, quinazoline, benzo[1,3]dioxole, 2,3-dihydrobenzo[1,4]dioxin, 4-H-benzo[1,3]dioxin or 3,4-dihydro-2H-benzo[b][1,4]dioxepine system.

The linkage to the heteroaryl radicals may be at any of the possible atoms; for example, pyridyl may be 2-, 3- or 4-pyridyl; thienyl may be 2- or 3-thienyl; furyl may be 2- or 3-furyl.

Also included are the corresponding N-oxides of these compounds, i.e., for example, 1-oxy-2-, -3- or -4-pyridyl.

The heteroaryl radicals may be mono- or polysubstituted by suitable groups as described above.

The invention also encompasses solvates or hydrates of the compounds of the formula I.

The compounds of the formula I are cannabinoid 1 receptor (CB1R) modulators and are, as such, suitable in humans and in animals for the treatment or for the prevention of diseases which are based on disruption of the endocannabinoid system.

For example, and without restriction, the compounds of the formula I are useful as psychotropic medicaments, especially for the treatment of psychiatric disorders including states of anxiety, depressions, disorders of the mind, insomnia, deliria, obsessive-compulsive neuroses, general psychoses, schizophrenia, attention deficit hyperactivity disorder (ADHD) in hyperkinetic children, and for the treatment of disorders in connection with the use of psychotropic substances, especially in the case of abuse of a substance and/or dependence on such a substance, including alcohol dependence and nicotine dependence, but also dependence on cocaine, methamphetamine and heroin (see, for example, Behavioural Pharmacology 2005, 16:275-296). Reviews of CBR1-mediated means of therapeutic intervention can be found, for example, in Ken Mackie: Annu Rev. Pharmacol. Toxicol. 46, 101-122 (2006), S. C. Black: Curr. Opin. Investig. Drugs 5, 389-394 (2004), V. Di Marzio et al.: Nat. Rev. Drug Discov. 3, 771-784 (2004), B. Le Foll et al.: J. Pharmacol. Exp. Ther. 312, 875-883 (2005) or L. Walter et al.: Br. J. Pharmacol. 141, 775-785 (2004).

The inventive compounds of the formula I may be used as medicaments for the treatment of migraine, stress, disorders of psychosomatic origin, panic attacks, epilepsy, disrupted movement, especially dyskinesias or Parkinson's disease, trembling and dystonia.

The inventive compounds of the formula I can also be used as medicaments for the treatment of disorders of memory, mental defects, especially for the treatment of age-related dementia, of Alzheimer's disease and for the treatment of reduced alertness or wakefulness.

In addition, it is also possible to use the compounds of the formula I as neuroprotectors, for the treatment of ischemia, cranial injuries and the treatment of neurodegenerative disorders, including chorea, Huntington's chorea, Tourette's syndrome.

The inventive compounds of the formula I can also be used as medicaments in the treatment of pain; this includes neuropathic pain, acute peripheral pain, chronic pain of inflammatory origin.

The inventive compounds of the formula I may also serve as medicaments for the treatment of eating disorders (for example binge eating disorders, anorexia and bulimia), for the treatment of addiction to confectionery, carbohydrates, drugs, alcohol or other addictive substances.

The inventive compounds of the formula I are particularly suitable for the treatment of obesity or of bulimia, and for the treatment of type II diabetes and also for the treatment of dyslipidemias and of metabolic syndrome. The inventive compounds of the formula I are therefore useful for the treatment of obesity and of the risks associated with obesity, especially the cardiovascular risks.

Moreover, the inventive compounds of the formula I may be used as medicaments for the treatment of gastrointestinal disorders, for the treatment of diarrhea, of gastric and intestinal ulcers, of vomiting, of bladder trouble and disorders of urination, of disorders of endocrine origin, of cardiovascular problems, of low blood pressure, of hemmorrhagic shock, of septic shock, chronic liver cirrhosis, liver steatosis, of nonalcoholic steatohepatitis, of asthma, of Raynaud's syndrome, of glaucoma, of fertility problems, termination of pregnancy, early birth, inflammatory symptoms, disorders of the immune system, especially autoimmune and neuroinflammatory disorders, for example rheumatic inflammation of joints, reactive arthritis, of disorders which lead to demyelinization, of multiple sclerosis, of infection disorders and viral disorders, for example encephalitis, ischemic stroke, and as medicaments for chemotherapy of cancer, for the treatment of Guillain-Barré syndrome and for the treatment of osteoporosis. The inventive compounds of the formula I may also find use as medicaments for the treatment of polycystic ovary syndrome (PCOS).

According to the present invention, the compounds of the formula I are particularly useful for the treatment of psychotic complaints, especially of schizophrenia, reduced alertness and hyperactivity (ADHD) in hyperkinetic children, for the treatment of eating disorders and of obesity, for the treatment of type II diabetes, for the treatment of deficits of memory and cognitive deficits, for the treatment of alcohol addiction, of nicotine addiction, i.e. for alcohol and tobacco withdrawal.

The inventive compounds of the formula I are very particularly useful for the treatment and prevention of eating disorders, appetite disorders, metabolic disorders, gastrointestinal disorders, inflammation symptoms, disorders of the immune system, psychotic disorders, alcohol addiction and nicotine addiction.

According to one of its aspects, the invention relates to the use of a compound of the formula I, the pharmaceutically acceptable salts thereof and the solvates or hydrates thereof for the treatment of the above-specified disorders and diseases.

The compound(s) of the formula I may also be administered in combination with further active ingredients.

The amount of a compound of the formula I which is required in order to achieve the desired biological effect is dependent upon a series of factors, for example the specific compound selected, the intended use, the mode of administration and the clinical condition of the patient. The daily dose is generally in the range from 0.3 mg to 100 mg (typically from 3 mg to 50 mg) per day per kilogram of bodyweight, for example 3-10 mg/kg/day. An intravenous dose may, for example, be in the range from 0.3 mg to 1.0 mg/kg and may suitably be administered as an infusion of from 10 ng to 100 ng per kilogram per minute. Suitable infusion solutions for these purposes may, for example, contain from 0.1 ng to 10 mg, typically from 1 ng to 10 mg, per milliliter. Single doses may contain, for example, from 1 mg to 10 g of the active ingredient. Ampoules for injections may therefore contain, for example, from 1 mg to 100 mg, and single dose formulations which can be administered orally, for example tablets or capsules, may contain, for example, from 1.0 to 1000 mg, typically from 10 to 600 mg. The compounds of the formula I may be used for therapy of the above-mentioned conditions as the compounds themselves, although they are preferably in the form of a pharmaceutical composition with an acceptable carrier. The carrier of course has to be acceptable, in the sense that it is compatible with the other constituents of the composition and is not damaging to the health of the patient. The carrier may be a solid or a liquid or both and is preferably formulated with the compound as a single dose, for example as a tablet, which may contain from 0.05 to 95% by weight of the active ingredient. Further pharmaceutically active substances may likewise be present, including further compounds of the formula I. The inventive pharmaceutical compositions may be produced by one of the known pharmaceutical methods which consist essentially in mixing the constituents with pharmacologically acceptable carriers and/or excipients.

Inventive pharmaceutical compositions are those which are suitable for oral, rectal, topical, peroral (for example sublingual) and parenteral (for example subcutaneous, intramuscular, intradermal or intravenous) administration, although the most suitable mode of administration depends in each individual case on the nature and severity of the condition to be treated and on the type of the compound of the formula I used in each case. Coated formulations and coated slow-release formulations are also encompassed by the scope of the invention. Preference is given to acid- and gastric fluid-resistant formulations. Suitable gastric fluid-resistant coatings include cellulose acetate phthalate, polyvinyl acetate phthalate, hydroxypropylmethylcellulose phthalate and anionic polymers of methacrylic acid and methyl methacrylate.

Suitable pharmaceutical preparations for oral administration may be in the form of separate units, for example capsules, cachets, lozenges or tablets, each of which contains a certain amount of the compound of the formula I; as powder or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional constituents) are brought into contact. In general, the compositions are prepared by uniform and homogeneous mixing of the active ingredient with a liquid carrier and/or finely divided solid carrier, after which the product is shaped if necessary. For example, a tablet can thus be produced by compressing or shaping a powder or granules of the compound, optionally with one or more additional constituents. Compressed tablets can be prepared by tableting the compound in free-flowing form, for example a powder or granules, optionally mixed with a binder, lubricant, inert diluent and/or one (or more) surfactants/dispersants in a suitable machine. Shaped tablets can be prepared by shaping the pulverulent compound moistened with an inert liquid diluent in a suitable machine.

Pharmaceutical compositions which are suitable for peroral (sublingual) administration include lozenges which contain a compound of the formula I with a flavoring, customarily sucrose, and gum arabic or tragacanth, and pastilles which include the compound in an inert base, such as gelatin and glycerol or sucrose and gum arabic.

Suitable pharmaceutical compositions for parenteral administration include preferably sterile aqueous preparations of a compound of the formula I which are preferably isotonic with the blood of the intended recipient. These preparations are preferably administered intravenously, although the administration may also be subcutaneous, intramuscular or intradermal as an injection. These preparations can preferably be produced by mixing the compound with water and making the solution obtained sterile and isotonic with the blood. Injectable compositions according to the invention generally contain from 0.1 to 5% by weight of the active compound.

Suitable pharmaceutical compositions for rectal administration are preferably in the form of single dose suppositories. These can be prepared by mixing a compound of the formula I with one or more conventional solid carriers, for example cocoa butter, and shaping the resulting mixture.

Suitable pharmaceutical compositions for topical application on the skin are preferably in the form of an ointment, cream, lotion, paste, spray, aerosol or oil. Useful carriers include petroleum jelly, lanolin, polyethylene glycols, alcohols and combinations of two or more of these substances. The active ingredient is generally present in a concentration of from 0.1 to 15% by weight of the composition, for example from 0.5 to 2%.

Transdermal administration is also possible. Suitable pharmaceutical compositions for transdermal applications may be in the form of single plasters which are suitable for long-term close contact with the epidermis of the patient. Such plasters suitably contain the active ingredient in an optionally buffered aqueous solution, dissolved and/or dispersed in an adhesive or dispersed in a polymer. A suitable active ingredient concentration is from approx. 1% to 35%, preferably from approx. 3% to 15%. A particular means of releasing the active ingredient may be by electrotransport or iontophoresis, as described, for example, in Pharmaceutical Research, 2(6): 318 (1986).

Further suitable active ingredients for the combination preparations are:

All antidiabetics which are mentioned in the Rote Liste 2007, chapter 12; all weight-reducing agents/appetite suppressants which are mentioned in the Rote Liste 2007, chapter 1; all diuretics which are mentioned in the Rote Liste 2007, chapter 36; all lipid-lowering agents which are mentioned in the Rote Liste 2007, chapter 58. They can be combined with the compound of the invention of the formula I in particular for a synergistic improvement in action. The active ingredient combination can be administered either by separate administration of the active ingredients to the patient or in the form of combination products in which a plurality of active ingredients are present in one pharmaceutical preparation. If the active ingredients are administered separately, this can be done simultaneously or successively. Most of the active ingredients mentioned hereinafter are disclosed in the USP Dictionary of USAN and International Drug Names, US Pharmacopeia, Rockville 2006.

Antidiabetics include insulin and insulin derivatives, for example Lantus® (see www.lantus.com) or HMR 1964 or Levemir® (insulin detemir), Humalog® (Insulin Lispro), Humulin®, VIAject™, SuliXen® or those as described in WO2005005477 (Novo Nordisk), fast-acting insulins (see U.S. Pat. No. 6,221,633), inhalable insulins, for example Exubera®, Nasulin™, or oral insulins, for example IN-105 (Nobex) or Oral-lyn™ (Generex Biotechnology), or Technosphere® Insulin (MannKind) or Cobalamin™ oral insulin, or insulins as described in WO2007128815, WO2007128817, WO2008034881, WO2008049711, or insulins which can be administered transdermally;

GLP-1 derivatives and GLP-1 agonists, for example exenatide or specific formulations thereof, as described, for example, in WO2008061355, liraglutide, taspoglutide (R-1583), albiglutide, lixisenatide or those which have been disclosed in WO 98/08871, WO2005027978, WO2006037811, WO2006037810 by Novo Nordisk A/S, in WO 01/04156 by Zealand or in WO 00/34331 by Beaufour-Ipsen, pramlintide acetate (Symlin; Amylin Pharmaceuticals), AVE-0010, BIM-51077 (R-1583, ITM-077), PC-DAC:Exendin-4 (an exendin-4 analog which is bonded covalently to recombinant human albumin), CVX-73, CVX-98 and CVx-96 (GLP-1 analogs which are bonded covalently to a monoclonal antibody which has specific binding sites for the GLP-1 peptide), CNTO-736 (a GLP-1 analog which is bonded to a domain which includes the Fc portion of an antibody), PGC-GLP-1 (GLP-1 bonded to a nanocarrier), agonists, as described, for example, in D. Chen et al., Proc. Natl. Acad. Sci. USA 104 (2007) 943, those as described in WO2006124529, WO2007124461, WO2008062457, WO2008082274, WO2008101017, WO2008081418, WO2008112939, WO2008112941, WO2008113601, WO2008116294, WO2008116648, WO2008119238, peptides, for example obinepitide (TM-30338), amylin receptor agonists, as described, for example, in WO2007104789, analogs of the human GLP-1, as described in WO2007120899, WO2008022015, WO2008056726, and orally active hypoglycemic ingredients.

Antidiabetics also include agonists of the glucose-dependent insulinotropic polypeptide (GIP) receptor, as described, for example, in WO2006121860.

Antidiabetics also include the glucose-dependent insulinotropic polypeptide (GIP), and also analogous compounds, as described, for example, in WO2008021560.

Antidiabetics also include analogs and derivatives of fibroblast growth factor 21 (FGF-21).

The orally active hypoglycemic ingredients preferably include

sulfonylureas,

biguanidines,

meglitinides,

oxadiazolidinediones,

thiazolidinediones,

PPAR and RXR modulators,

glucosidase inhibitors,

inhibitors of glycogen phosphorylase,

glucagon receptor antagonists,

glucokinase activators,

inhibitors of fructose 1,6-bisphosphatase

modulators of glucose transporter 4 (GLUT4),

inhibitors of glutamine-fructose-6-phosphate amidotransferase (GFAT),

GLP-1 agonists,

potassium channel openers, for example pinacidil, cromakalim, diazoxide, or those as described in R. D. Carr et al., Diabetes 52, 2003, 2513.2518, in J. B. Hansen et al., Current Medicinal Chemistry 11, 2004, 1595-1615, in T. M. Tagmose et al., J. Med. Chem. 47, 2004, 3202-3211 or in M. J. Coghlan et al., J. Med. Chem. 44, 2001, 1627-1653, or those which have been disclosed in WO 97/26265 and WO 99/03861 by Novo Nordisk A/S, active ingredients which act on the ATP-dependent potassium channel of the beta cells,

inhibitors of dipeptidylpeptidase IV (DPP-IV),

insulin sensitizers,

inhibitors of liver enzymes involved in stimulating gluconeogenesis and/or glycogenolysis,

modulators of glucose uptake, of glucose transport and of glucose reabsorption,

modulators of sodium-dependent glucose transporter 1 or 2 (SGLT1, SGLT2),

inhibitors of 11-beta-hydroxysteroid dehydrogenase-1 (11β-HSD1),

inhibitors of protein tyrosine phosphatase 1B (PTP-1B),

nicotinic acid receptor agonists,

inhibitors of hormone-sensitive or endothelial lipases,

inhibitors of acetyl-CoA carboxylase (ACC1 and/or ACC2) or

inhibitors of GSK-3 beta.

Also included are compounds which modify the metabolism, such as active antihyperlipidemic ingredients and active antilipidemic ingredients,

HMGCoA reductase inhibitors,

farnesoid X receptor (FXR) modulators,

fibrates,

cholesterol reabsorption inhibitors,

CETP inhibitors,

bile acid reabsorption inhibitors,

MTP inhibitors,

agonists of estrogen receptor gamma (ERRγ agonists),

sigma-1 receptor antagonists,

antagonists of the somatostatin 5 receptor (SSTS receptor);

compounds which reduce food intake, and

compounds which increase thermogenesis.

In one embodiment of the invention, the compound of the formula I is administered in combination with insulin.

In one embodiment, the compound of the formula I is administered in combination with an active ingredient which acts on the ATP-dependent potassium channel of the beta cells, for example sulfonylureas, for example tolbutamide, glibenclamide, glipizide, gliclazide or glimepiride.

In one embodiment, the compound of the formula I is administered in combination with a tablet which comprises both glimepiride, which is released rapidly, and metformin, which is released over a longer period (as described, for example, in US2007264331, WO2008050987, WO2008062273).

In one embodiment, the compound of the formula I is administered in combination with a biguanide, for example metformin.

In another embodiment, the compound of the formula I is administered in combination with a meglitinide, for example repaglinide, nateglinide or mitiglinide.

In a further embodiment, the compound of the formula I is administered with a combination of mitiglinide with a glitazone, e.g. pioglitazone hydrochloride.

In a further embodiment, the compound of the formula I is administered with a combination of mitiglinide with an alpha-glucosidase inhibitor.

In a further embodiment, the compound of the formula I is administered in combination with antidiabetic compounds, as described in WO2007095462, WO2007101060, WO2007105650.

In a further embodiment, the compound of the formula I is administered in combination with antihypoglycemic compounds, as described in WO2007137008, WO2008020607.

In one embodiment, the compound of the formula I is administered in combination with a thiazolidinedione, for example troglitazone, ciglitazone, pioglitazone, rosiglitazone or the compounds disclosed in WO 97/41097 by Dr. Reddy's Research Foundation, especially 5-[[4-[(3,4-dihydro-3-methyl-4-oxo-2-quinazolinylmethoxy]phenyl]methyl]-2,4-thiazolidinedione.

In one embodiment of the invention, the compound of the formula I is administered in combination with a PPAR gamma agonist, for example rosiglitazone, pioglitazone, JTT-501, Gl 262570, R-483, CS-011 (rivoglitazone), DRL-17564, DRF-2593 (balaglitazone), INT-131, T-2384, or those as described in WO2005086904, WO2007060992, WO2007100027, WO2007103252, WO2007122970, WO2007138485, WO2008006319, WO2008006969, WO2008010238, WO2008017398, WO2008028188, WO2008066356, WO2008084303, WO2008089461-WO2008089464, WO2008093639, WO2008096769, WO2008096820, WO2008096829, US2008194617, WO2008099944, WO2008108602, WO2008109334, WO2008126731, WO2008126732.

In one embodiment of the invention, the compound of the formula I is administered in combination with Competact™, a solid combination of pioglitazone hydrochloride with metformin hydrochloride.

In one embodiment of the invention, the compound of the formula I is administered in combination with Tandemact™, a solid combination of pioglitazone with glimepiride.

In a further embodiment of the invention, the compound of the formula I is administered in combination with a solid combination of pioglitazone hydrochloride with an angiotensin II agonist, for example TAK-536.

In one embodiment of the invention, the compound of the formula I is administered in combination with a PPAR alpha agonist or mixed PPAR alpha/PPAR delta agonist, for example GW9578, GW-590735, K-111, LY-674, KRP-101, DRF-10945, LY-518674, CP-900691, BMS-687453, BMS-711939, or those as described in WO2001040207, WO2002096894, WO2005097076, WO2007056771, WO2007087448, WO2007089667, WO2007089557, WO2007102515, WO2007103252, JP2007246474, WO2007118963, WO2007118964, WO2007126043, WO2008006043, WO2008006044, WO2008012470, WO2008035359, WO2008087365, WO2008087366, WO2008087367, WO2008117982.

In one embodiment of the invention, the compound of the formula I is administered in combination with a mixed PPAR alpha/gamma agonist, for example naveglitazar, LY-510929, ONO-5129, E-3030, AVE 8042, AVE 8134, AVE 0847, CKD-501 (lobeglitazone sulfate), MBX-213, KY-201 or as described in WO 00/64888, WO 00/64876, WO03/020269, WO2004024726, WO2007099553, US2007276041, WO2007085135, WO2007085136, WO2007141423, WO2008016175, WO2008053331, WO2008109697, WO2008109700, WO2008108735 or in J. P. Berger et al., TRENDS in Pharmacological Sciences 28(5), 244-251, 2005.

In one embodiment of the invention, the compound of the formula I is administered in combination with a PPAR delta agonist, for example GW-501516, or as described in WO2006059744, WO2006084176, WO2006029699, WO2007039172-WO2007039178, WO2007071766, WO2007101864, US2007244094, WO2007119887, WO2007141423, US2008004281, WO2008016175, WO2008066356, WO2008071311, WO2008084962, US2008176861.

In one embodiment of the invention, the compound of the formula I is administered in combination with a pan-SPPARM (selective PPAR modulator alpha, gamma, delta), for example GFT-505, or those as described in WO2008035359.

In one embodiment, the compound of the formula I is administered in combination with metaglidasen or with MBX-2044 or other partial PPAR gamma agonists/antagonists.

In one embodiment, the compound of the formula I is administered in combination with an α-glucosidase inhibitor, for example miglitol or acarbose, or those as described, for example, in WO2007114532, WO2007140230, US2007287674, US2008103201, WO2008065796, WO2008082017.

In one embodiment, the compound of the formula I is administered in combination with an inhibitor of glycogen phosphorylase, for example PSN-357 or FR-258900, or those as described in WO2003084922, WO2004007455, WO2005073229-31, WO2005067932, WO2008062739, WO2008099000, WO2008113760.

In one embodiment, the compound of the formula I is administered in combination with glucagon receptor antagonists, for example A-770077 or NNC-25-2504 or as described in WO2004100875, WO2005065680, WO2006086488, WO2007047177, WO2007106181, WO2007111864, WO2007120270, WO2007120284, WO2007123581, WO2007136577, WO2008042223, WO2008098244.

In a further embodiment, the compound of the formula I is administered in combination with an antisense compound, e.g. ISIS-325568, which inhibits the production of the glucagon receptor.

In one embodiment, the compound of the formula I is administered in combination with activators of glucokinase, for example LY-2121260 (WO2004063179), PSN-105, PSN-110, GKA-50, or those as described, for example, in WO2004072031, WO2004072066, WO2005080360, WO2005044801, WO2006016194, WO2006058923, WO2006112549, WO2006125972, WO2007017549, WO2007017649, WO2007007910, WO2007007040-42, WO2007006760-61, WO2007006814, WO2007007886, WO2007028135, WO2007031739, WO2007041365, WO2007041366, WO2007037534, WO2007043638, WO2007053345, WO2007051846, WO2007051845, WO2007053765, WO2007051847, WO2007061923, WO2007075847, WO2007089512, WO2007104034, WO2007117381, WO2007122482, WO2007125103, WO2007125105, US2007281942, WO2008005914, WO2008005964, WO2008043701, WO2008044777, WO2008047821, US2008096877, WO2008050117, WO2008050101, WO2008059625, US2008146625, WO2008078674, WO2008079787, WO2008084043, WO2008084044, WO2008084872, WO2008089892, WO2008091770, WO2008075073, WO2008084043, WO2008084044, WO2008084872, WO2008084873, WO2008089892, WO2008091770, JP2008189659, WO2008104994, WO2008111473, WO2008116107, WO2008118718, WO2008120754.

In one embodiment, the compound of the formula I is administered in combination with an inhibitor of gluconeogenesis, as described, for example, in FR-225654, WO2008053446.

In one embodiment, the compound of the formula I is administered in combination with inhibitors of fructose 1,6-bisphosphatase (FBPase), for example MB-07729, CS-917 (MB-06322) or MB-07803, or those as described in WO2006023515, WO2006104030, WO2007014619, WO2007137962, WO2008019309, WO2008037628.

In one embodiment, the compound of the formula I is administered in combination with modulators of glucose transporter 4 (GLUT4), for example KST-48 (D.-O. Lee et al.: Arzneim.-Forsch. Drug Res. 54 (12), 835 (2004)).

In one embodiment, the compound of the formula I is administered in combination with inhibitors of glutamine: fructose-6-phosphate amidotransferase (GFAT), as described, for example, in WO2004101528.

In one embodiment, the compound of the formula I is administered in combination with inhibitors of dipeptidyl peptidase IV (DPP-IV), for example vildagliptin (LAF-237), sitagliptin (MK-0431), sitagliptin phosphate, saxagliptin ((BMS-477118), GSK-823093, PSN-9301, SYR-322, SYR-619, TA-6666, TS-021, GRC-8200 (melogliptin), GW-825964X, KRP-104, DP-893, ABT-341, ABT-279 or another salt thereof, S-40010, S-40755, PF-00734200, BI-1356, PHX-1149, alogliptin benzoate, linagliptin, melogliptin, or those compounds as described in WO2003074500, WO2003106456, WO2004037169, WO200450658, WO2005037828, WO2005058901, WO2005012312, WO2005/012308, WO2006039325, WO2006058064, WO2006015691, WO2006015701, WO2006015699, WO2006015700, WO2006018117, WO2006099943, WO2006099941, JP2006160733, WO2006071752, WO2006065826, WO2006078676, WO2006073167, WO2006068163, WO2006085685, WO2006090915, WO2006104356, WO2006127530, WO2006111261, US2006890898, US2006803357, US2006303661, WO2007015767 (LY-2463665), WO2007024993, WO2007029086, WO2007063928, WO2007070434, WO2007071738, WO2007071576, WO2007077508, WO2007087231, WO2007097931, WO2007099385, WO2007100374, WO2007112347, WO2007112669, WO2007113226, WO2007113634, WO2007115821, WO2007116092, US2007259900, EP1852108, US2007270492, WO2007126745, WO2007136603, WO2007142253, WO2007148185, WO2008017670, US2008051452, WO2008027273, WO2008028662, WO2008029217, JP2008031064, JP2008063256, WO2008033851, WO2008040974, WO2008040995, WO2008060488, WO2008064107, WO2008066070, WO2008077597, JP2008156318, WO2008087560, WO2008089636, WO2008093960, WO2008096841, WO2008101953, WO2008118848, WO2008119005, WO2008119208, WO2008120813, WO2008121506.

In one embodiment, the compound of the formula I is administered in combination with Janumet™, a solid combination of sitagliptin phosphate with metformin hydrochloride.

In one embodiment, the compound of the formula I is administered in combination with Eucreas®, a solid combination of vildagliptin with metformin hydrochloride.

In a further embodiment, the compound of the formula I is administered in combination with a solid combination of alogliptin benzoate with pioglitazone.

In one embodiment, the compound of the formula I is administered in combination with a solid combination of a salt of sitagliptin with metformin hydrochloride.

In one embodiment, the compound of the formula I is administered in combination with a combination of a DPP-IV inhibitor with omega-3 fatty acids or omega-3 fatty acid esters, as described, for example, in WO2007128801.

In one embodiment, the compound of the formula I is administered in combination with a solid combination of a salt of sitagliptin with metformin hydrochloride.

In one embodiment, the compound of the formula I is administered in combination with a substance which enhances insulin secretion, for example KCP-265 (WO2003097064), or those as described in WO2007026761, WO2008045484, US2008194617.

In one embodiment, the compound of the formula I is administered in combination with agonists of the glucose-dependent insulinotropic receptor (GDIR), for example APD-668.

In one embodiment of the invention, the compound of the formula I is administered in combination with an ATP citrate lyase inhibitor, for example SB-204990.

In one embodiment, the compound of the formula I is administered in combination with modulators of the sodium-dependent glucose transporter 1 or 2 (SGLT1, SGLT2), for example KGA-2727, T-1095, SGL-0010, AVE 2268, SAR 7226, SGL-5083, SGL-5085, SGL-5094, ISIS-388626, sergliflozin or dapagliflozin, or as described, for example, in WO2004007517, WO200452903, WO200452902, PCT/EP2005/005959, WO2005085237, JP2004359630, WO2005121161, WO2006018150, WO2006035796, WO2006062224, WO2006058597, WO2006073197, WO2006080577, WO2006087997, WO2006108842, WO2007000445, WO2007014895, WO2007080170, WO2007093610, WO2007126117, WO2007128480, WO2007129668, US2007275907, WO2007136116, WO2007143316, WO2007147478, WO2008001864, WO2008002824, WO2008013277, WO2008013280, WO2008013321, WO2008013322, WO2008016132, WO2008020011, JP2008031161, WO2008034859, WO2008042688, WO2008044762, WO2008046497, WO2008049923, WO2008055870, WO2008055940, WO2008069327, WO2008070609, WO2008071288, WO2008072726, WO2008083200, WO2008090209, WO2008090210, WO2008101586, WO2008101939, WO2008116179, WO2008116195, US2008242596 or by A. L. Handlon in Expert Opin. Ther. Patents (2005) 15(11), 1531-1540.

In one embodiment, the compound of the formula I is administered in combination with inhibitors of 11-beta-hydroxysteroid dehydrogenase 1 (11β-HSD1), for example BVT-2733, JNJ-25918646, NCB-13739, INCB-20817, DIO-92 ((−)-ketoconazole) or those as described, for example, in WO200190090-94, WO200343999, WO2004112782, WO200344000, WO200344009, WO2004112779, WO2004113310, WO2004103980, WO2004112784, WO2003065983, WO2003104207, WO2003104208, WO2004106294, WO2004011410, WO2004033427, WO2004041264, WO2004037251, WO2004056744, WO2004058730, WO2004065351, WO2004089367, WO2004089380, WO2004089470-71, WO2004089896, WO2005016877, WO2005063247, WO2005097759, WO2006010546, WO2006012227, WO2006012173, WO2006017542, WO2006034804, WO2006040329, WO2006051662, WO2006048750, WO2006049952, WO2006048331, WO2006050908, WO2006024627, WO2006040329, WO2006066109, WO2006074244, WO2006078006, WO2006106423, WO2006132436, WO2006134481, WO2006134467, WO2006135795, WO2006136502, WO2006138508, WO2006138695, WO2006133926, WO2007003521, WO2007007688, US2007066584, WO2007029021, WO2007047625, WO2007051811, WO2007051810, WO2007057768, WO2007058346, WO2007061661, WO2007068330, WO2007070506, WO2007087150, WO2007092435, WO2007089683, WO2007101270, WO2007105753, WO2007107470, WO2007107550, WO2007111921, US2007207985, US2007208001, WO2007115935, WO2007118185, WO2007122411, WO2007124329, WO2007124337, WO2007124254, WO2007127688, WO2007127693, WO2007127704, WO2007127726, WO2007127763, WO2007127765, WO2007127901, US2007270424, JP2007291075, WO2007130898, WO2007135427, WO2007139992, WO2007144394, WO2007145834, WO2007145835, WO2007146761, WO2008000950, WO2008000951, WO2008003611, WO2008005910, WO2008006702, WO2008006703, WO2008011453, WO2008012532, WO2008024497, WO2008024892, WO2008032164, WO2008034032, WO2008043544, WO2008044656, WO2008046758, WO2008052638, WO2008053194, WO2008071169, WO2008074384, WO2008076336, WO2008076862, WO2008078725, WO2008087654, WO2008088540, WO2008099145, WO2008101885, WO2008101886, WO2008101907, WO2008101914, WO2008106128, WO2008110196, WO2008119017, WO2008120655, WO2008127924.

In one embodiment, the compound of the formula I is administered in combination with inhibitors of protein tyrosine phosphatase 1B (PTP-1B), as described, for example, in WO200119830-31, WO200117516, WO2004506446, WO2005012295, WO2005116003, WO2005116003, WO2006007959, DE 10 2004 060542.4, WO2007009911, WO2007028145, WO2007067612-615, WO2007081755, WO2007115058, US2008004325, WO2008033455, WO2008033931, WO2008033932, WO2008033934, WO2008089581.

In one embodiment of the invention, the compound of the formula I is administered in combination with an agonist of GPR109A (HM74A receptor agonists; NAR agonists (nicotinic acid receptor agonists)), for example nicotinic acid or “extended release niacin” in conjunction with MK-0524A (laropiprant) or MK-0524, or those compounds as described in WO2004041274, WO2006045565, WO2006045564, WO2006069242, WO2006085108, WO2006085112, WO2006085113, WO2006124490, WO2006113150, WO2007017261, WO2007017262, WO2007017265, WO2007015744, WO2007027532, WO2007092364, WO2007120575, WO2007134986, WO2007150025, WO2007150026, WO2008016968, WO2008051403, WO2008086949, WO2008091338, WO2008097535, WO2008099448, US2008234277, WO2008127591.

In another embodiment of the invention, the compound of the formula I is administered in combination with a solid combination of niacin with simvastatin.

In another embodiment of the invention, the compound of the formula I is administered in combination with nicotinic acid or “extended release niacin” in conjunction with MK-0524A (laropiprant).

In a further embodiment of the invention, the compound of the formula I is administered in combination with nicotinic acid or “extended release niacin” in conjunction with MK-0524A (laropiprant) and with simvastatin.

In one embodiment of the invention, the compound of the formula I is administered in combination with nicotinic acid or another nicotinic acid receptor agonist and a prostaglandin DP receptor antagonist, for example those as described in WO2008039882.

In another embodiment of the invention, the compound of the formula I is administered in combination with an agonist of GPR116, as described, for example, in WO2006067531, WO2006067532.

In one embodiment, the compound of the formula I is administered in combination with modulators of GPR40, as described, for example, in WO2007013689, WO2007033002, WO2007106469, US2007265332, WO2007123225, WO2007131619, WO2007131620, WO2007131621, US2007265332, WO2007131622, WO2007136572, WO2008001931, WO2008030520, WO2008030618, WO2008054674, WO2008054675, WO2008066097, US2008176912.

In one embodiment, the compound of the formula I is administered in combination with modulators of GPR119 (G-protein-coupled glucose-dependent insulinotropic receptor), for example PSN-119-1, PSN-821, PSN-119-2, MBX-2982 or those as described, for example, in WO2004065380, WO2005061489 (PSN-632408), WO2006083491, WO2007003960-62 and WO2007003964, WO2007035355, WO2007116229, WO2007116230, WO2008005569, WO2008005576, WO2008008887, WO2008008895, WO2008025798, WO2008025799, WO2008025800, WO2008070692, WO2008076243, WO200807692, WO2008081204, WO2008081205, WO2008081206, WO2008081207, WO2008081208, WO2008083238, WO2008085316, WO2008109702.

In a further embodiment, the compound of the formula I is administered in combination with modulators of GPR120, as described, for example, in EP1688138, WO2008066131, WO2008066131, WO2008103500, WO2008103501.

In one embodiment, the compound of the formula I is administered in combination with inhibitors of hormone-sensitive lipase (HSL) and/or phospholipases, as described, for example, in WO2005073199, WO2006074957, WO2006087309, WO2006111321, WO2007042178, WO2007119837, WO2008122352, WO2008122357.

In one embodiment, the compound of the formula I is administered in combination with inhibitors of endothelial lipase, as described, for example, in WO2007110216.

In one embodiment, the compound of the formula I is administered in combination with a phospholipase A2 inhibitor, for example darapladib or A-002, or those as described in WO2008048866, WO20080488867.

In one embodiment, the compound of the formula I is administered in combination with myricitrin, a lipase inhibitor (WO2007119827).

In one embodiment, the compound of the formula I is administered in combination with an inhibitor of glycogen synthase kinase-3 beta (GSK-3 beta), as described, for example, in US2005222220, WO2005085230, WO2005111018, WO2003078403, WO2004022544, WO2003106410, WO2005058908, US2005038023, WO2005009997, US2005026984, WO2005000836, WO2004106343, EP1460075, WO2004014910, WO2003076442, WO2005087727, WO2004046117, WO2007073117, WO2007083978, WO2007120102, WO2007122634, WO2007125109, WO2007125110, US2007281949, WO2008002244, WO2008002245, WO2008016123, WO2008023239, WO2008044700, WO2008056266, WO2008057940, WO2008077138, EP1939191, EP1939192, WO2008078196, WO2008094992, WO2008112642, WO2008112651, WO2008113469, WO2008121063, WO2008121064.

In one embodiment, the compound of the formula I is administered in combination with an inhibitor of phosphoenolpyruvate carboxykinase (PEPCK), for example those as described in WO2004074288.

In one embodiment, the compound of the formula I is administered in combination with an inhibitor of phosphoinositide kinase-3 (PI3K), for example those as described in WO2008027584, WO2008070150, WO2008125833, WO2008125835, WO2008125839.

In one embodiment, the compound of the formula I is administered in combination with an inhibitor of serum/glucocorticoid-regulated kinase (SGK), as described, for example, in WO2006072354, WO2007093264, WO2008009335, WO2008086854.

In one embodiment, the compound of the formula I is administered in combination with a modulator of the glucocorticoid receptor, as described, for example, in WO2008057855, WO2008057856, WO2008057857, WO2008057859, WO2008057862, WO2008059867, WO2008059866, WO2008059865, WO2008070507, WO2008124665, WO2008124745.

In one embodiment, the compound of the formula I is administered in combination with a modulator of the mineralocorticoid receptor (MR), for example drospirenone, or those as described in WO2008104306, WO2008119918.

In one embodiment, the compound of the formula I is administered in combination with an inhibitor of protein kinase C beta (PKC beta), for example ruboxistaurin, or those as described in WO2008096260, WO2008125945.

In one embodiment, the compound of the formula I is administered in combination with an inhibitor of protein kinase D, for example doxazosin (WO2008088006).

In a further embodiment, the compound of the formula I is administered in combination with an activator of the AMP-activated protein kinase (AMPK), as described, for example, in WO2007062568, WO2008006432, WO2008016278, WO2008016730, WO2008083124.

In one embodiment, the compound of the formula I is administered in combination with an inhibitor of ceramide kinase, as described, for example, in WO2007112914, WO2007149865.

In a further embodiment, the compound of the formula I is administered in combination with an inhibitor of MAPK-interacting kinase 1 or 2 (MNK1 or 2), as described, for example, in WO2007104053, WO2007115822, WO2008008547, WO2008075741.

In one embodiment, the compound of the formula I is administered in combination with inhibitors of “I-kappaB kinase” (IKK inhibitors), as described, for example, in WO2001000610, WO2001030774, WO2004022057, WO2004022553, WO2005097129, WO2005113544, US2007244140, WO2008099072, WO2008099073, WO2008099073, WO2008099074, WO2008099075.

In another embodiment, the compound of the formula I is administered in combination with inhibitors of NF-kappaB (NFKB) activation, for example salsalate.

In a further embodiment, the compound of the formula I is administered in combination with inhibitors of ASK-1 (apoptosis signal-regulating kinase 1), as described, for example, in WO2008016131.

In one embodiment of the invention, the compound of the formula I is administered in combination with an HMG-CoA reductase inhibitor such as simvastatin, fluvastatin, pravastatin, lovastatin, atorvastatin, cerivastatin, rosuvastatin, pitavastatin, L-659699, BMS-644950, or those as described in US2007249583, WO2008083551.

In a further embodiment of the invention, the compound of the formula I is administered in combination with a farnesoid X receptor (FXR) modulator, for example WAY-362450 or those as described in WO2003099821, WO2005056554, WO2007052843, WO2007070796, WO2007092751, JP2007230909, WO2007095174, WO2007140174, WO2007140183, WO2008000643, WO2008002573, WO2008025539, WO2008025540, JP2008214222.

In another embodiment of the invention, the compound of the formula I is administered in combination with a ligand of the liver X receptor (LXR), as described, for example, in WO2007092965, WO2008041003, WO2008049047, WO2008065754, WO2008073825, US2008242677.

In one embodiment of the invention, the compound of the formula I is administered in combination with a fibrate, for example fenofibrate, clofibrate, bezafibrate, or those as described in WO2008093655.

In one embodiment of the invention, the compound of the formula I is administered in combination with fibrates, for example the choline salt of fenofibrate (SLV-348).

In one embodiment of the invention, the compound of the formula I is administered in combination with fibrates, for example the choline salt of fenofibrate and an HMG-CoA reductase inhibitor, for example rosuvastatin.

In a further embodiment of the invention, the compound of the formula I is administered in combination with bezafibrate and diflunisal.

In a further embodiment of the invention, the compound of the formula I is administered in combination with a solid combination of fenofibrate or a salt thereof with simvastatin, rosuvastatin, fluvastatin, lovastatin, cerivastatin, pravastatin, pitavastatin or atorvastatin.

In a further embodiment of the invention, the compound of the formula I is administered in combination with Synordia®, a solid combination of fenofibrate with metformin.

In one embodiment of the invention, the compound of the formula I is administered in combination with a cholesterol reabsorption inhibitor, for example ezetimibe, tiqueside, pamaqueside, FM-VP4 (sitostanol/campesterol ascorbyl phosphate; Forbes Medi-Tech, WO2005042692, WO2005005453), MD-0727 (Microbia Inc., WO2005021497, WO2005021495) or with compounds as described in WO2002066464, WO2005000353 (Kotobuki Pharmaceutical Co. Ltd.) or WO2005044256 or WO2005062824 (Merck & Co.) or WO2005061451 and WO2005061452 (AstraZeneca AB) and WO2006017257 (Phenomix) or WO2005033100 (Lipideon Biotechnology AG), or as described in WO2002050060, WO2002050068, WO2004000803, WO2004000804, WO2004000805, WO2004087655, WO2004097655, WO2005047248, WO2006086562, WO2006102674, WO2006116499, WO2006121861, WO2006122186, WO2006122216, WO2006127893, WO2006137794, WO2006137796, WO2006137782, WO2006137793, WO2006137797, WO2006137795, WO2006137792, WO2006138163, WO2007059871, US2007232688, WO2007126358, WO2008033431, WO2008033465, WO2008052658, WO2008057336, WO2008085300.

In one embodiment of the invention, the compound of the formula I is administered in combination with an NPC1L1 antagonist, for example those as described in WO2008033464, WO2008033465.

In one embodiment of the invention, the compound of the formula I is administered in combination with Vytorin™, a solid combination of ezetimibe with simvastatin.

In one embodiment of the invention, the compound of the formula I is administered in combination with a solid combination of ezetimibe with atorvastatin.

In one embodiment of the invention, the compound of the formula I is administered in combination with a solid combination of ezetimibe with fenofibrate.

In one embodiment of the invention, the further active ingredient is a diphenylazetidinone derivative, as described, for example, in U.S. Pat. No. 6,992,067 or U.S. Pat. No. 7,205,290.

In a further embodiment of the invention, the further active ingredient is a diphenylazetidinone derivative, as described, for example, in U.S. Pat. No. 6,992,067 or U.S. Pat. No. 7,205,290, combined with a statin, for example simvastatin, fluvastatin, pravastatin, lovastatin, cerivastatin, atorvastatin, pitavastatin or rosuvastatin.

In one embodiment of the invention, the compound of the formula I is administered in combination with a solid combination of lapaquistat, a squalene synthase inhibitor, with atorvastatin.

In one embodiment of the invention, the compound of the formula I is administered in combination with a CETP inhibitor, for example torcetrapib, anacetrapib or JTT-705 (dalcetrapib), or those as described in WO2006002342, WO2006010422, WO2006012093, WO2006073973, WO2006072362, WO2007088996, WO2007088999, US2007185058, US2007185113, US2007185154, US2007185182, WO2006097169, WO2007041494, WO2007090752, WO2007107243, WO2007120621, US2007265252, US2007265304, WO2007128568, WO2007132906, WO2008006257, WO2008009435, WO2008018529, WO2008058961, WO2008058967, WO2008059513, WO2008070496, WO2008115442, WO2008111604.

In one embodiment of the invention, the compound of the formula I is administered in combination with bile acid reabsorption inhibitors (inhibitors of the intestinal bile acid transporter (IBAT)) (see, for example, U.S. Pat. No. 6,245,744, U.S. Pat. No. 6,221,897 or WO00/61568), for example HMR 1741, or those as described in DE 10 2005 033099.1 and DE 10 2005 033100.9, DE 10 2006 053635, DE 10 2006 053637, WO2007009655-56, WO2008058628, WO2008058629, WO2008058630, WO2008058631.

In one embodiment, the compound of the formula I is administered in combination with agonists of GPBAR1 (G-protein-coupled bile acid receptor-1; TGR5), as described, for example, in US20060199795, WO2007110237, WO2007127505, WO2008009407, WO2008067219, WO2008067222, FR2908310, WO2008091540, WO2008097976.

In one embodiment of the invention, the compound of the formula I is administered in combination with inhibitors of the TRPM5 channel (TRP cation channel M5), as described, for example, in WO2008097504.

In one embodiment of the invention, the compound of the formula I is administered in combination with a polymeric bile acid adsorber, for example cholestyramine, colesevelam hydrochloride.

In one embodiment of the invention, the compound of the formula I is administered in combination with colesevelam hydrochloride and metformin or a sulfonylurea or insulin.

In one embodiment of the invention, the compound of the formula I is administered in combination with a chewing gum comprising phytosterols (Reductol™)

In one embodiment of the invention, the compound of the formula I is administered in combination with an inhibitor of the microsomal triglyceride transfer protein (MTP inhibitor), for example implitapide, BMS-201038, R-103757, AS-1552133, SLx-4090, AEGR-733, or those as described in WO2005085226, WO2005121091, WO2006010423, WO2006113910, WO2007143164, WO2008049806, WO2008049808, WO2008090198, WO2008100423.

In a further embodiment of the invention, the compound of the formula I is administered in combination with a combination of a cholesterol absorption inhibitor, for example ezetimibe, and an inhibitor of the triglyceride transfer protein (MTP inhibitor), for example implitapide, as described in WO2008030382 or in WO2008079398.

In one embodiment of the invention, the compound of the formula I is administered in combination with an active antihypertriglyceridemic ingredient, for example those as described in WO2008032980.

In another embodiment of the invention, the compound of the formula I is administered in combination with an antagonist of the somatostatin 5 receptor (SSTS receptor), for example those as described in WO2006094682.

In one embodiment of the invention, the compound of the formula I is administered in combination with an ACAT inhibitor, for example avasimibe, SMP-797 or KY-382, or those as described in WO2008087029, WO2008087030, WO2008095189.

In a further embodiment of the invention, the compound of the formula I is administered in combination with an inhibitor of liver carnitine palmitoyltransferase 1 (L-CPT1), as described, for example, in WO2007063012, WO2007096251 (ST-3473), WO2008015081, US2008103182, WO2008074692.

In a further embodiment of the invention, the compound of the formula I is administered in combination with a modulator of serine palmitoyltransferase (SPT), as described, for example, in WO2008031032, WO2008046071, WO2008083280, WO2008084300.

In one embodiment of the invention, the compound of the formula I is administered in combination with a squalene synthetase inhibitor, for example BMS-188494, TAK-475 (lapaquistat acetate), or as described in WO2005077907, JP2007022943, WO2008003424.

In one embodiment of the invention, the compound of the formula I is administered in combination with ISIS-301012 (mipomersen), an antisense oligonucleotide which is capable of regulating the apolipoprotein B gene.

In one embodiment of the invention, the compound of the formula I is administered in combination with a stimulator of the ApoA-1 gene, as described, for example in WO2008092231.

In one embodiment of the invention, the compound of the formula I is administered in combination with an LDL receptor inducer (see U.S. Pat. No. 6,342,512), for example HMR1171, HMR1586, or those as described in WO2005097738, WO2008020607.

In another embodiment of the invention, the compound of the formula I is administered in combination with an HDL cholesterol-elevating agent, for example those as described in WO2008040651, WO2008099278.

In one embodiment of the invention, the compound of the formula I is administered in combination with an ABCA1 expression enhancer, as described, for example, in WO2006072393, WO2008062830.

In one embodiment of the invention, the compound of the formula I is administered in combination with a lipoprotein lipase modulator, for example ibrolipim (NO-1886).

In one embodiment of the invention, the compound of the formula I is administered in combination with a lipoprotein(a) antagonist, for example gemcabene (CI-1027).

In one embodiment of the invention, the compound of the formula I is administered in combination with a lipase inhibitor, for example orlistat or cetilistat (ATL-962).

In one embodiment of the invention, the compound of the formula I is administered in combination with an adenosine A1 receptor agonist (adenosine A1 R), as described, for example, in EP1258247, EP1375508, WO2008028590, WO2008077050.

In one embodiment of the invention, the compound of the formula I is administered in combination with an adenosine A2B receptor agonist (adenosine A2B R), for example ATL-801.

In another embodiment of the invention, the compound of the formula I is administered in combination with a modulator of adenosine A2A and/or adenosine A3 receptors, as described, for example, in WO2007111954, WO2007121918, WO2007121921, WO2007121923, WO2008070661.

In a further embodiment of the invention, the compound of the formula I is administered in combination with an agonist of the adenosine A1/A2B receptors, as described, for example, in WO2008064788, WO2008064789.

In one embodiment of the invention, the compound of the formula I is administered in combination with an adenosine A2B receptor antagonist (adenosine A2B R), as described in US2007270433, WO2008027585, WO2008080461.

In one embodiment, the compound of the formula I is administered in combination with inhibitors of acetyl-CoA carboxylase (ACC1 and/or ACC2), for example those as described in WO199946262, WO200372197, WO2003072197, WO2005044814, WO2005108370, JP2006131559, WO2007011809, WO2007011811, WO2007013691, WO2007095601-603, WO2007119833, WO2008065508, WO2008069500, WO2008070609, WO2008072850, WO2008079610, WO2008088688, WO2008088689, WO2008088692, US2008171761, WO2008090944, JP2008179621, US2008200461, WO2008102749, WO2008103382, WO2008121592.

In another embodiment, the compound of the formula I is administered in combination with modulators of microsomal acyl-CoA:glycerol-3-phosphate acyltransferase 3 (GPAT3, described in WO2007100789) or with modulators of microsomal acyl-CoA:glycerol-3-phosphate acyltransferase 4 (GPAT4, described in WO2007100833).

In a further embodiment, the compound of the formula I is administered in combination with modulators of xanthine oxidoreductase (XOR).

In another embodiment, the compound of the formula I is administered in combination with inhibitors of soluble epoxide hydrolase (sEH), as described, for example, in WO2008051873, WO2008051875, WO2008073623, WO2008094869, WO2008112022.

In a further embodiment, the compound of the formula I is administered in combination with CART modulators (see “Cocaine-amphetamine-regulated transcript influences energy metabolism, anxiety and gastric emptying in mice” Asakawa, A. et al.: Hormone and Metabolic Research (2001), 33(9), 554-558);

NPY antagonists, for example N-{-4-[(4-aminoquinazolin-2-ylamino)methyl]-cyclohexylmethyl}naphthalene-1-sulfonamide hydrochloride (CGP 71683A) or velneperit;

NPY-5 receptor antagonists, such as L-152804 or the compound “NPY-5-BY” from Banyu, or as described, for example, in WO2006001318, WO2007103295, WO2007125952, WO2008026563, WO2008026564, WO2008052769, WO2008092887, WO2008092888, WO2008092891;

NPY-4 receptor antagonists, as described, for example, in WO2007038942;

NPY-2 receptor antagonists, as described, for example, in WO2007038943;

peptide YY 3-36 (PYY3-36) or analogous compounds, for example CJC-1682 (PYY3-36 conjugated with human serum albumin via Cys34) or CJC-1643 (derivative of PYY3-36, which is conjugated in vivo to serum albumin), or those as described in WO2005080424, WO2006095166, WO2008003947;

derivatives of the peptide obestatin, as described by WO2006096847;

CB1R (cannabinoid receptor 1) antagonists, for example rimonabant, surinabant (SR147778), SLV-319 (ibipinabant), AVE-1625, taranabant (MK-0364) or salts thereof, otenabant (CP-945,598), rosonabant, V-24343 or those compounds as described in, for example, EP 0656354, WO 00/15609, WO2001/64632-64634, WO 02/076949, WO2005080345, WO2005080328, WO2005080343, WO2005075450, WO2005080357, WO200170700, WO2003026647-48, WO200302776, WO2003040107, WO2003007887, WO2003027069, U.S. Pat. No. 6,509,367, WO200132663, WO2003086288, WO2003087037, WO2004048317, WO2004058145, WO2003084930, WO2003084943, WO2004058744, WO2004013120, WO2004029204, WO2004035566, WO2004058249, WO2004058255, WO2004058727, WO2004069838, US20040214837, US20040214855, US20040214856, WO2004096209, WO2004096763, WO2004096794, WO2005000809, WO2004099157, US20040266845, WO2004110453, WO2004108728, WO2004000817, WO2005000820, US20050009870, WO200500974, WO2004111033-34, WO200411038-39, WO2005016286, WO2005007111, WO2005007628, US20050054679, WO2005027837, WO2005028456, WO2005063761-62, WO2005061509, WO2005077897, WO2006018662, WO2006047516, WO2006060461, WO2006067428, WO2006067443, WO2006087480, WO2006087476, WO2006100208, WO2006106054, WO2006111849, WO2006113704, WO2007009705, WO2007017124, WO2007017126, WO2007018459, WO2007018460, WO2007016460, WO2007020502, WO2007026215, WO2007028849, WO2007031720, WO2007031721, WO2007036945, WO2007038045, WO2007039740, US20070015810, WO2007046548, WO2007047737, WO2007057687, WO2007062193, WO2007064272, WO2007079681, WO2007084319, WO2007084450, WO2007086080, EP1816125, US2007213302, WO2007095513, WO2007096764, US2007254863, WO2007119001, WO2007120454, WO2007121687, WO2007123949, US2007259934, WO2007131219, WO2007133820, WO2007136571, WO2007136607, WO2007136571, U.S. Pat. No. 7,297,710, WO2007138050, WO2007139464, WO2007140385, WO2007140439, WO2007146761, WO2007148061, WO2007148062, US2007293509, WO2008004698, WO2008017381, US2008021031, WO2008024284, WO2008031734, WO2008032164, WO2008034032, WO2008035356, WO2008036021, WO2008036022, WO2008039023, WO2998043544, WO2008044111, WO2008048648, EP1921072-A1, WO2008053341, WO2008056377, WO2008059207, WO2008059335, WO2008062424, WO2008068423, WO2008068424, WO2008070305, WO2008070306, WO2008074816, WO2008074982, WO2008075012, WO2008075013, WO2008075019, WO2008075118, WO2008076754, WO2008081009, WO2008084057, EP1944295, US2008090809, US2008090810, WO2008092816, WO2008094473, WO2008094476, WO2008099076, WO2008099139, WO2008101995, US2008207704, WO2008107179, WO2008109027, WO2008112674, WO2008115705, WO2008118414, WO2008119999, WO200812000, WO2008121257, WO2008127585;

cannabinoid receptor 1/cannabinoid receptor 2 (CB1/CB2) modulating compounds, for example delta-9-tetrahydrocannabivarin, or those as described, for example, in WO2007001939, WO2007044215, WO2007047737, WO2007095513, WO2007096764, WO2007112399, WO2007112402, WO2008122618;

modulators of FAAH (fatty acid amide hydrolase), as described, for example, in WO2007140005, WO2008019357, WO2008021625, WO2008023720, WO2008030532;

inhibitors of fatty acid synthase (FAS), as described, for example, in WO2008057585, WO2008059214, WO2008075064, WO2008075070, WO2008075077;

inhibitors of LCE (long chain fatty acid elongase), as described, for example, in WO2008120653;

vanilloid-1 receptor modulators (modulators of TRPV1), as described, for example, in WO2007091948, WO2007129188, WO2007133637, WO2008007780, WO2008010061, WO2008007211, WO2008010061, WO2008015335, WO2008018827, WO2008024433, WO2008024438, WO2008032204, WO2008050199, WO2008059339, WO2008059370, WO2008066664, WO2008075150, WO2008090382, WO2008090434, WO2008093024, WO2008107543, WO2008107544, WO2008110863;

modulators, antagonists or inverse agonists of the opioid receptors, for example GSK-982 or those as described, for example, in WO2007047397, WO2008021849, WO2008021851, WO2008032156, WO2008059335;

modulators of the “orphan opioid (ORL-1) receptor”, as described, for example, in US2008249122, WO2008089201;

agonists of the prostaglandin receptor, for example bimatoprost or those compounds as described in WO2007111806;

MC4 receptor agonists (melanocortin-4 receptor agonists, MC4R agonists, for example N-[2-(3a-benzyl-2-methyl-3-oxo-2,3,3a,4,6,7-hexahydropyrazolo[4,3-c]pyridin-5-yl)-1-(4-chlorophenyl)-2-oxoethyl]-1-amino-1,2,3,4-tetrahydronaphthalene-2-carboxamide; (WO 01/91752)) or LB53280, LB53279, LB53278 or THIQ, MB243, RY764, CHIR-785, PT-141, MK-0493, or those as described in WO2005060985, WO2005009950, WO2004087159, WO2004078717, WO2004078716, WO2004024720, US20050124652, WO2005051391, WO2004112793, WOUS20050222014, US20050176728, US20050164914, US20050124636, US20050130988, US20040167201, WO2004005324, WO2004037797, WO2005042516, WO2005040109, WO2005030797, US20040224901, WO200501921, WO200509184, WO2005000339, EP1460069, WO2005047253, WO2005047251, WO2005118573, EP1538159, WO2004072076, WO2004072077, WO2006021655-57, WO2007009894, WO2007015162, WO2007041061, WO2007041052, JP2007131570, EP-1842846, WO2007096186, WO2007096763, WO2007141343, WO2008007930, WO2008017852, WO2008039418, WO2008087186, WO2008087187, WO2008087189, WO2008087186-WO2008087190, WO2008090357;

orexin receptor 1 antagonists (OX1R antagonists), orexin receptor 2 antagonists (OX2R antagonists) or mixed OX1R/OX2R antagonists (e.g. 1-(2-methylbenzoxazol-6-yl)-3-[1,5]naphthyridin-4-ylurea hydrochloride (SB-334867-A), or those as described, for example, in WO200196302, WO200185693, WO2004085403, WO2005075458, WO2006067224, WO2007085718, WO2007088276, WO2007116374; WO2007122591, WO2007126934, WO2007126935, WO2008008517, WO2008008518, WO2008008551, WO2008020405, WO2008026149, WO2008038251, US2008132490, WO2008065626, WO2008078291, WO2008087611, WO2008081399, WO2008108991, WO2008107335, US2008249125);

histamine H3 receptor antagonists/inverse agonists (e.g. 3-cyclohexyl-1-(4,4-dimethyl-1,4,6,7-tetrahydroimidazo[4,5-c]pyridin-5-yl)propan-1-one oxalic acid salt (WO 00/63208), or those as described in WO200064884, WO2005082893, US2005171181 (e.g. PF-00389027), WO2006107661, WO2007003804, WO2007016496, WO2007020213, WO2007049798, WO2007055418, WO2007057329, WO2007065820, WO2007068620, WO2007068641, WO2007075629, WO2007080140, WO2007082840, WO2007088450, WO2007088462, WO2007094962, WO2007099423, WO2007100990, WO2007105053, WO2007106349, WO2007110364, WO2007115938, WO2007131907, WO2007133561, US2007270440, WO2007135111, WO2007137955, US2007281923, WO2007137968, WO2007138431, WO2007146122, WO2008005338, WO2008012010, WO2008015125, WO2008045371, EP1757594, WO2008068173, WO2008068174, US20080171753, WO2008072703, WO2008072724, US2008188484, US2008188486, US2008188487, WO2008109333, WO2008109336);

histamine H1/histamine H3 modulators, for example betahistine or its dihydrochloride;

modulators of the histamine H3 transporter or of the histamine H3/serotonin transporter, as described, for example, in WO2008002816, WO2008002817, WO2008002818, WO2008002820;

histamine H4 modulators, as described, for example, in WO2007117399;

CRF antagonists (e.g. [2-methyl-9-(2,4,6-trimethylphenyl)-9H-1,3,9-triazafluoren-4-yl]dipropylamine (WO 00/66585) or those CRF1 antagonists as described in WO2007105113, WO2007133756, WO2008036541, WO2008036579, WO2008083070);

CRF BP antagonists (e.g. urocortin);

urocortin agonists;

modulators of the beta-3 adrenoceptor, for example 1-(4-chloro-3-methanesulfonylmethylphenyl)-2-[2-(2,3-dimethyl-1H-indol-6-yloxy)ethylamino]ethanol hydrochloride (WO 01/83451) or solabegron (GW-427353) or N-5984 (KRP-204), or those as described in JP2006111553, WO2002038543, WO2002038544, WO2007048840-843, WO2008015558, EP1947103;

MSH (melanocyte-stimulating hormone) agonists;

MCH (melanine-concentrating hormone) receptor antagonists (for example NBI-845, A-761, A-665798, A-798, ATC-0175, T-226296, T-71 (AMG-071, AMG-076), GW-856464, NGD-4715, ATC-0453, ATC-0759, GW-803430, or those compounds as described in WO2005085200, WO2005019240, WO2004011438, WO2004012648, WO2003015769, WO2004072025, WO2005070898, WO2005070925, WO2004039780, WO2004092181, WO2003033476, WO2002006245, WO2002089729, WO2002002744, WO2003004027, FR2868780, WO2006010446, WO2006038680, WO2006044293, WO2006044174, JP2006176443, WO2006018280, WO2006018279, WO2006118320, WO2006130075, WO2007018248, WO2007012661, WO2007029847, WO2007024004, WO2007039462, WO2007042660, WO2007042668, WO2007042669, US2007093508, US2007093509, WO2007048802, JP2007091649, WO2007092416; WO2007093363-366, WO2007114902, WO2007114916, WO2007141200, WO2007142217, US2007299062, WO2007146758, WO2007146759, WO2008001160, WO2008016811, WO2008020799, WO2008022979, WO2008038692, WO2008041090, WO2008044632, WO2008047544, WO2008061109, WO2008065021, WO2008068265, WO2008071646, WO2008076562, JP2008088120, WO2008086404, WO2008086409);

CCK-A (CCK-1) agonists/modulators (for example {2-[4-(4-chloro-2,5-dimethoxyphenyl)-5-(2-cyclohexylethyl)thiazol-2-ylcarbamoyl]-5,7-dimethylindol-1-yl}acetic acid trifluoroacetic acid salt (WO 99/15525) or SR-146131 (WO 0244150) or SSR-125180), or those as described in WO2005116034, WO2007120655, WO2007120688, WO2007120718, WO2008091631;

serotonin reuptake inhibitors (e.g. dexfenfluramine), or those as described in WO2007148341, WO2008034142, WO2008081477, WO2008120761;

mixed serotonin/dopamine reuptake inhibitors (e.g. bupropion), or those as described in WO2008063673, or solid combinations of bupropion with naltrexone or bupropion with zonisamide;

mixed reuptake inhibitors, for example DOV-21947;

mixed serotoninergic and noradrenergic compounds (e.g. WO 00/71549);

5-HT receptor agonists, for example 1-(3-ethylbenzofuran-7-yl)piperazine oxalic acid salt (WO 01/09111);

mixed dopamine/norepinephrine/acetylcholine reuptake inhibitors (e.g. tesofensine), or those as described, for example, in WO2006085118;

dopamine antagonists, as described, for example, in WO2008079838, WO2008079839, WO2008079847, WO2008079848;

norepinephrine reuptake inhibitors, as described, for example, in US2008076724;

5-HT2A receptor antagonists, as described, for example, in WO2007138343;

5-HT2C receptor agonists (for example lorcaserine hydrochloride (APD-356) or BVT-933, or those as described in WO200077010, WO200077001-02, WO2005019180, WO2003064423, WO200242304, WO2005035533, WO2005082859, WO2006004937, US2006025601, WO2006028961, WO2006077025, WO2006103511, WO2007028132, WO2007084622, US2007249709; WO2007132841, WO2007140213, WO2008007661, WO2008007664, WO2008009125, WO2008010073, WO2008108445);

5-HT6 receptor modulators, for example E-6837, BVT-74316 or PRX-07034, or those as described, for example, in WO2005058858, WO2007054257, WO2007107373, WO2007108569, WO2007108742-744, WO2008003703, WO2008027073, WO2008034815, WO2008054288, EP1947085, WO2008084491, WO2008084492, WO2008092665, WO2008092666, WO2008101247, WO2008110598, WO2008116831, WO2008116833;

agonists of estrogen receptor gamma (ERRγ agonists), as described, for example, in WO2007131005, WO2008052709;

agonists of estrogen receptor alpha (ERRα/ERR1 agonists), as described, for example, in WO2008109727;

sigma-1 receptor antagonists, as described, for example, in WO2007098953, WO2007098961, WO2008015266, WO2008055932, WO2008055933;

muscarin 3 receptor (M3R) antagonists, as described, for example, in WO2007110782, WO2008041184;

bombesin receptor agonists (BRS-3 agonists), as described, for example, in WO2008051404, WO2008051405, WO2008051406, WO2008073311;

galanin receptor antagonists;

growth hormone (e.g. human growth hormone or AOD-9604);

growth hormone releasing compounds (tert-butyl 6-benzyloxy-1-(2-diisopropylaminoethylcarbamoyl)-3,4-dihydro-1H-isoquinoline-2-carboxylate (WO 01/85695));

growth hormone secretagogue receptor antagonists (ghrelin antagonists), for example A-778193, or those as described in WO2005030734, WO2007127457, WO2008008286;

growth hormone secretagogue receptor modulators (ghrelin modulators), for example JMV-2959, JMV-3002, JMV-2810, JMV-2951, or those as described in WO2006012577 (e.g. YIL-781 or YIL-870), WO2007079239, WO2008092681;

TRH agonists (see, for example, EP 0 462 884);

decoupling protein 2 or 3 modulators;

chemical decouplers (e.g. WO2008059023, WO2008059024, WO2008059025, WO2008059026);

leptin agonists (see, for example, Lee, Daniel W.; Leinung, Matthew C.; Rozhayskaya-Arena, Marina; Grasso, Patricia. Leptin agonists as a potential approach to the treatment of obesity. Drugs of the Future (2001), 26(9), 873-881);

DA agonists (bromocriptin, doprexin);

lipase/amylase inhibitors (e.g. WO 00/40569, WO2008107184);

inhibitors of diacylglycerol O-acyltransferases (DGATs), for example BAY-74-4113, or as described, for example, in US2004/0224997, WO2004094618, WO200058491, WO2005044250, WO2005072740, JP2005206492, WO2005013907, WO2006004200, WO2006019020, WO2006064189, WO2006082952, WO2006120125, WO2006113919, WO2006134317, WO2007016538, WO2007060140, JP2007131584, WO2007071966, WO2007126957, WO2007137103, WO2007137107, WO2007138304, WO2007138311, WO2007141502, WO2007141517, WO2007141538, WO2007141545, WO2007144571, WO2008011130, WO2008011131, WO2008039007, WO2008048991, WO2008067257, WO2008099221;

inhibitors of monoacylglycerol acyltransferase (2-acylglycerol O-acyltransferase; MGAT), as described, for example, in WO2008038768;

inhibitors of fatty acid synthase (FAS), for example C75, or those as described in WO2004005277, WO2008006113;

inhibitors of stearoyl-CoA delta9 desaturase (SCD1), as described, for example, in WO2007009236, WO2007044085, WO2007046867, WO2007046868, WO20070501124, WO2007056846, WO2007071023, WO2007130075, WO2007134457, WO2007136746, WO2007143597, WO2007143823, WO2007143824, WO2008003753, WO2008017161, WO2008024390, WO2008029266, WO2008036715, WO2008043087, WO2008044767, WO2008046226, WO2008056687, WO2008062276, WO2008064474, WO2008074824, WO2008074832, WO2008074833, WO2008074834, WO2008074835, WO2008089580, WO2008096746, WO2008104524, WO2008116898, US2008249100, WO2008120744, WO2008120759, WO2008123469, WO2008127349;

inhibitors of fatty acid desaturase 1 (deltas desaturase), as described, for example, in WO2008089310;

hypoglycemic/hypertriglyceridemic indoline compounds, as described in WO2008039087;

inhibitors of “adipocyte fatty acid-binding protein aP2”, for example BMS-309403;

activators of adiponectin secretion, as described, for example, in WO2006082978, WO2008105533;

promoters of adiponectin secretion, as described, for example, in WO2007125946, WO2008038712;

modified adiponectins, as described, for example, in WO2008121009;

oxyntomodulin or analogs thereof;

oleoyl-estrone

or agonists or partial agonists of the thyroid hormone receptor (thyroid hormone receptor agonists), for example: KB-2115 (eprotirome), QRX-431 (sobetirome) or DITPA, or those as described in WO20058279, WO200172692, WO200194293, WO2003084915, WO2004018421, WO2005092316, WO2007003419, WO2007009913, WO2007039125, WO2007110225, WO2007110226, WO2007128492, WO2007132475, WO2007134864, WO2008001959, WO2008106213;

or agonists of the thyroid hormone receptor beta (TR-beta), for example MB-07811 or MB-07344, or those as described in WO2008062469.

In one embodiment of the invention, the compound of the formula I is administered in combination with a combination of eprotirome with ezetimibe.

In one embodiment of the invention, the compound of the formula I is administered in combination with an inhibitor of site-1 protease (SIP), for example PF-429242.

In a further embodiment of the invention, the compound of the formula I is administered in combination with a modulator of the “trace amine associated receptor 1” (TAAR1), as described, for example, in US2008146523, WO2008092785.

In one embodiment of the invention, the compound of the formula I is administered in combination with an inhibitor of growth factor receptor bound protein 2 (GRB2), as described, for example, in WO2008067270.

In a further embodiment of the invention, the compound of the formula I is administered in combination with an RNAi (siRNA) therapeutic agent directed against PCSK9 (proprotein convertase subtilisin/kexin type 9).

In one embodiment, the compound of the formula I is administered in combination with Omacor® or Lovaza™ (omega-3 fatty acid ester; highly concentrated ethyl ester of eicosapentaenoic acid and of docosahexaenoic acid).

In one embodiment, the compound of the formula I is administered in combination with lycopene.

In one embodiment of the invention, the compound of the formula I is administered in combination with an antioxidant, for example OPC-14117, AGI-1067 (succinobucol), probucol, tocopherol, ascorbic acid, β-carotene or selenium.

In one embodiment of the invention, the compound of the formula I is administered in combination with a vitamin, for example vitamin B6 or vitamin B12.

In one embodiment, the compound of the formula I is administered in combination with more than one of the aforementioned compounds, for example in combination with a sulfonylurea and metformin, a sulfonylurea and acarbose, repaglinide and metformin (PrandiMet™), insulin and a sulfonylurea, insulin and metformin, insulin and troglitazone, insulin and lovastatin, etc.

In another embodiment, the compound of the formula I is administered in combination with an inhibitor of carboanhydrase type 2 (carbonic anhydrase type 2), for example those as described in WO2007065948.

In another embodiment, the compound of the formula I is administered in combination with topiramat or a derivative thereof, as described in WO2008027557.

In a further embodiment, the compound of the formula I is administered in combination with a solid combination of topiramat with phentermin (Qnexa™)

In a further embodiment, the compound of the formula I is administered in combination with an antisense compound, e.g. ISIS-377131, which inhibits the production of the glucocorticoid receptor.

In another embodiment, the compound of the formula I is administered in combination with an aldosterone synthase inhibitor and an antagonist of the glucocorticoid receptor, a cortisol synthesis inhibitor and/or an antagonist of the corticotropin releasing factor, as described, for example, in EP1886695, WO2008119744.

In one embodiment, the compound of the formula I is administered in combination with an agonist of the RUP3 receptor, as described, for example, in WO2007035355, WO2008005576.

In another embodiment, the compound of the formula I is administered in combination with an activator of the gene which codes for ataxia telangiectasia mutated (ATM) protein kinase, for example chloroquine.

In one embodiment, the compound of the formula I is administered in combination with a tau protein kinase 1 inhibitor (TPK1 inhibitor), as described, for example, in WO2007119463.

In one embodiment, the compound of the formula I is administered in combination with a “c-Jun N-terminal kinase” inhibitor (JNK inhibitor), as described, for example, in WO2007125405, WO2008028860, WO2008118626.

In one embodiment, the compound of the formula I is administered in combination with an endothelin A receptor antagonist, for example avosentan (SPP-301).

In one embodiment, the compound of the formula I is administered in combination with modulators of the glucocorticoid receptor (GR), for example KB-3305 or those compounds as described, for example, in WO2005090336, WO2006071609, WO2006135826, WO2007105766, WO2008120661.

In one embodiment, the further active ingredient is varenicline tartrate, a partial agonist of the alpha 4-beta 2 nicotinic acetylcholine receptor.

In one embodiment, the further active ingredient is trodusquemine.

In one embodiment, the further active ingredient is a modulator of the enzyme SIRT1 and/or SIRT3 (an NAD⁺-dependent protein deacetylase); this active ingredient may, for example, be resveratrol in suitable formulations, or those compounds as specified in WO2007019416 (e.g. SRT-1720), WO2008073451.

In one embodiment of the invention, the further active ingredient is DM-71 (N-acetyl-L-cysteine with bethanechol).

In one embodiment, the compound of the formula I is administered in combination with antihypercholesterolemic compounds, as described, for example, in WO2007107587, WO2007111994, WO2008106600, WO2008113796.

In a further embodiment, the compound of the formula I is administered in combination with inhibitors of SREBP (sterol regulatory element-binding protein), as described, for example, in WO2008097835.

In another embodiment, the compound of the formula I is administered in combination with a cyclic peptide agonist of the VPAC2 receptor, as described, for example, in WO2007101146, WO2007133828.

In a further embodiment, the compound of the formula I is administered in combination with an agonist of the endothelin receptor, as described, for example, in WO2007112069.

In a further embodiment, the compound of the formula I is administered in combination with AKP-020 (bis(ethylmaltolato)oxovanadium(IV)).

In another embodiment, the compound of the formula I is administered in combination with tissue-selective androgen receptor modulators (SARM), as described, for example, in WO2007099200, WO2007137874.

In a further embodiment, the compound of the formula I is administered in combination with an AGE (advanced glycation endproduct) inhibitor, as described, for example, in JP2008024673.

In one embodiment of the invention, the further active ingredient is leptin;

see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

In another embodiment of the invention, the further active ingredient is metreleptin (recombinant methionyl-leptin) combined with pramlintide.

In a further embodiment of the invention, the further active ingredient is the tetrapeptide ISF-402.

In one embodiment, the further active ingredient is dexamphetamine or amphetamine.

In one embodiment, the further active ingredient is fenfluramine or dexfenfluramine.

In another embodiment, the further active ingredient is sibutramine or those derivatives as described in WO2008034142.

In one embodiment, the further active ingredient is mazindol or phentermin.

In a further embodiment, the further active ingredient is geniposidic acid (WO2007100104) or derivatives thereof (JP2008106008).

In one embodiment, the further active ingredient is a nasal calcium channel blocker, for example diltiazem, or those as described in U.S. Pat. No. 7,138,107.

In one embodiment, the further active ingredient is an inhibitor of sodium-calcium ion exchange, for example those as described in WO2008028958, WO2008085711.

In a further embodiment, the further active ingredient is a blocker of calcium channels, for example of CaV3.2 or CaV2.2, as described in WO2008033431, WO2008033447, WO2008033356, WO2008033460, WO2008033464, WO2008033465, WO2008033468, WO2008073461.

In one embodiment, the further active ingredient is a modulator of a calcium channel, for example those as described in WO2008073934, WO2008073936.

In one embodiment, the further active ingredient is a blocker of the “T-type calcium channel”, as described, for example, in WO2008033431, WO2008110008.

In one embodiment, the further active ingredient is an inhibitor of KCNQ potassium channel 2 or 3, for example those as described in US2008027049, US2008027090.

In one embodiment, the further active ingredient is an inhibitor of the potassium Kv1.3 ion channel, for example those as described in WO2008040057, WO2008040058, WO2008046065.

In another embodiment, the further active ingredient is a modulator of the MCP-1 receptor (monocyte chemoattractant protein-1 (MCP-1)), for example those as described in WO2008014360, WO2008014381.

In one embodiment, the further active ingredient is a modulator of somatostatin receptor 5 (SSTR5), for example those as described in WO2008019967, US2008064697, US2008249101, WO2008000692.

In one embodiment, the further active ingredient is a modulator of somatostatin receptor 2 (SSTR2), for example those as described in WO2008051272.

In one embodiment, the further active ingredient is an erythropoietin-mimetic peptide which acts as an erythropoietin (EPO) receptor agonist. Such molecules are described, for example, in WO2008042800.

In a further embodiment, the further active ingredient is an anorectic/a hypoglycemic compound, for example those as described in WO2008035305, WO2008035306, WO2008035686.

In one embodiment, the further active ingredient is an inductor of lipoic acid synthetase, for example those as described in WO2008036966, WO2008036967.

In one embodiment, the further active ingredient is a stimulator of endothelial nitric oxide synthase (eNOS), for example those as described in WO2008058641, WO2008074413.

In one embodiment, the further active ingredient is a modulator of carbohydrate and/or lipid metabolism, for example those as described in WO2008059023, WO2008059024, WO2008059025, WO2008059026.

In a further embodiment, the further active ingredient is an angiotensin II receptor antagonist, for example those as described in WO2008062905, WO2008067378.

In one embodiment, the further active ingredient is an agonist of the sphingosine-1-phosphate receptor (SIP), for example those as described in WO2008064315, WO2008074820, WO2008074821.

In one embodiment, the further active ingredient is an agent which retards gastric emptying, for example 4-hydroxyisoleucine (WO2008044770).

In one embodiment, the further active ingredient is a muscle-relaxing substance, as described, for example, in WO2008090200.

In a further embodiment, the further active ingredient is an inhibitor of monoamine oxidase B (MAO-B), for example those as described in WO2008092091.

In another embodiment, the further active ingredient is an inhibitor of the binding of cholesterol and/or triglycerides to the SCP-2 protein (sterol carrier protein-2), for example those as described in US2008194658.

In another embodiment, the further active ingredient is lisofylline, which prevents autoimmune damage to insulin-producing cells.

In one embodiment, the compound of the formula I is administered in combination with bulking agents, preferably insoluble bulking agents (see, for example, Carob/Caromax® (Zunft H J; et al., Carob pulp preparation for treatment of hypercholesterolemia, ADVANCES IN THERAPY (2001 September-October), 18(5), 230-6). Caromax is a carob-containing product from Nutrinova, Nutrition Specialties & Food Ingredients GmbH, Industriepark Hochst, 65926 Frankfurt/Main)). Combination with Caromax® is possible in one preparation or by separate administration of compounds of the formula I and Caromax®. Caromax® can in this connection also be administered in the form of food products such as, for example, in bakery products or muesli bars.

It will be appreciated that every suitable combination of the compounds of the invention with one or more of the aforementioned compounds and optionally one or more other pharmacologically active substances is considered to be covered within the scope of protection conferred by the present invention.

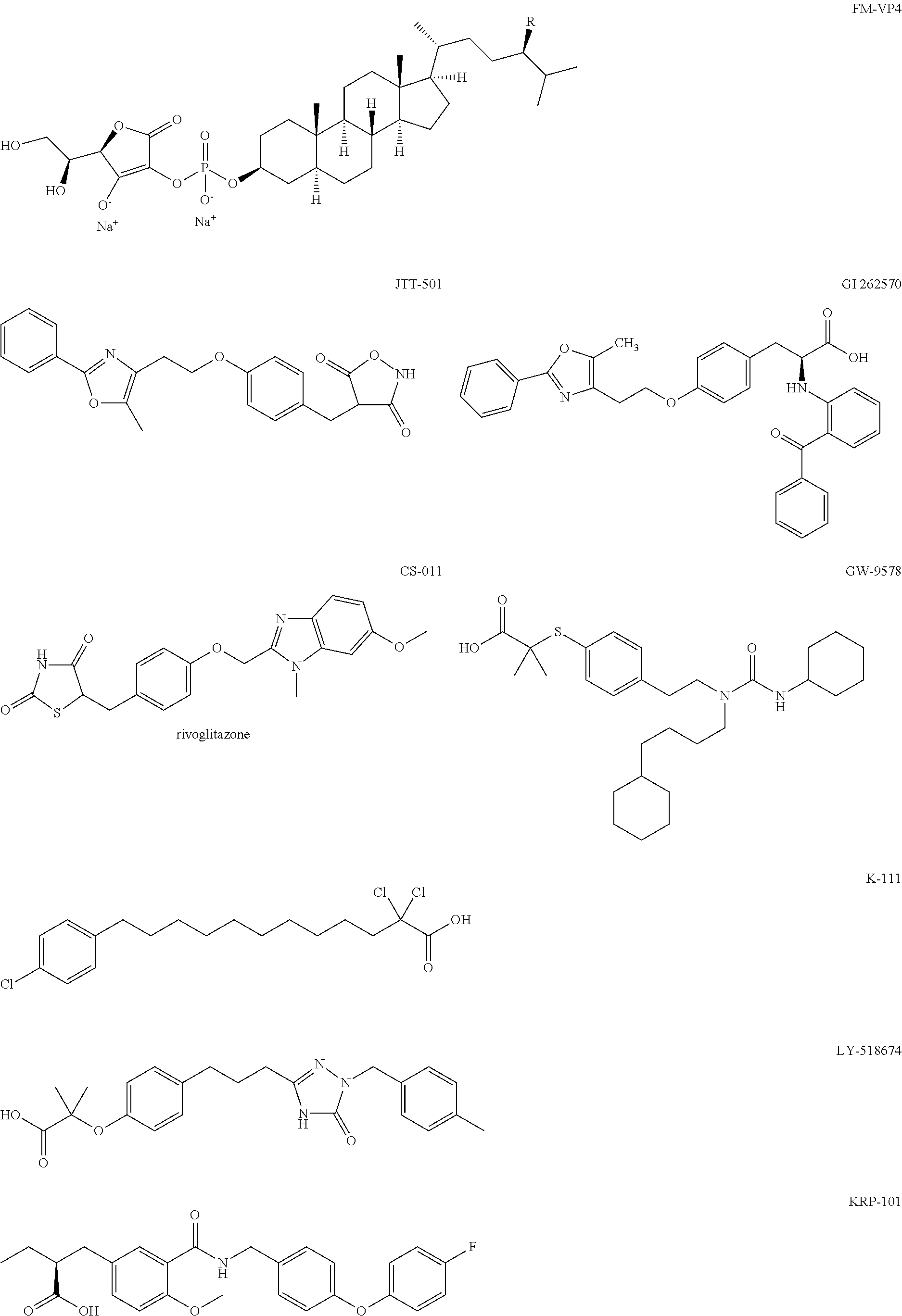

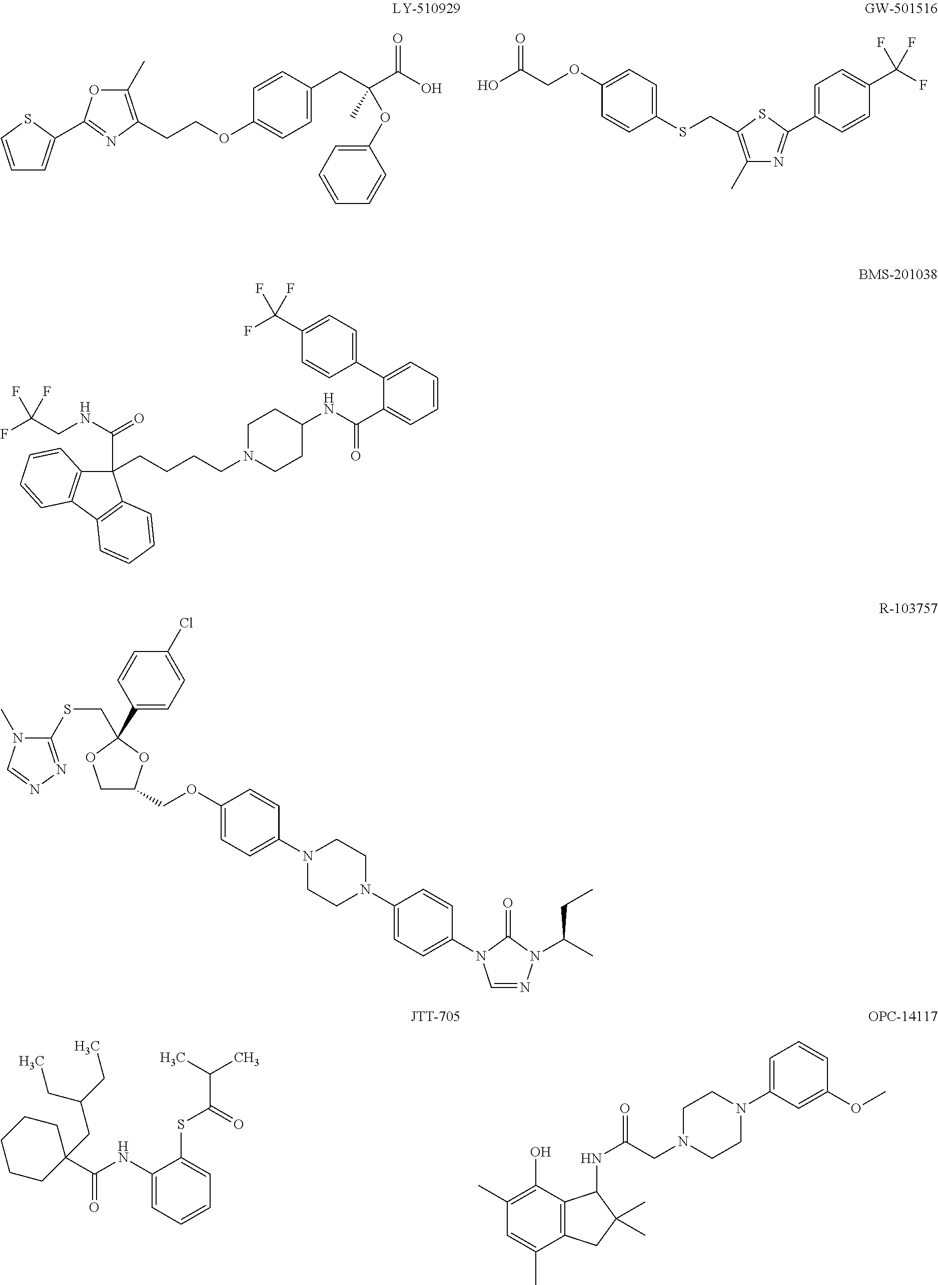

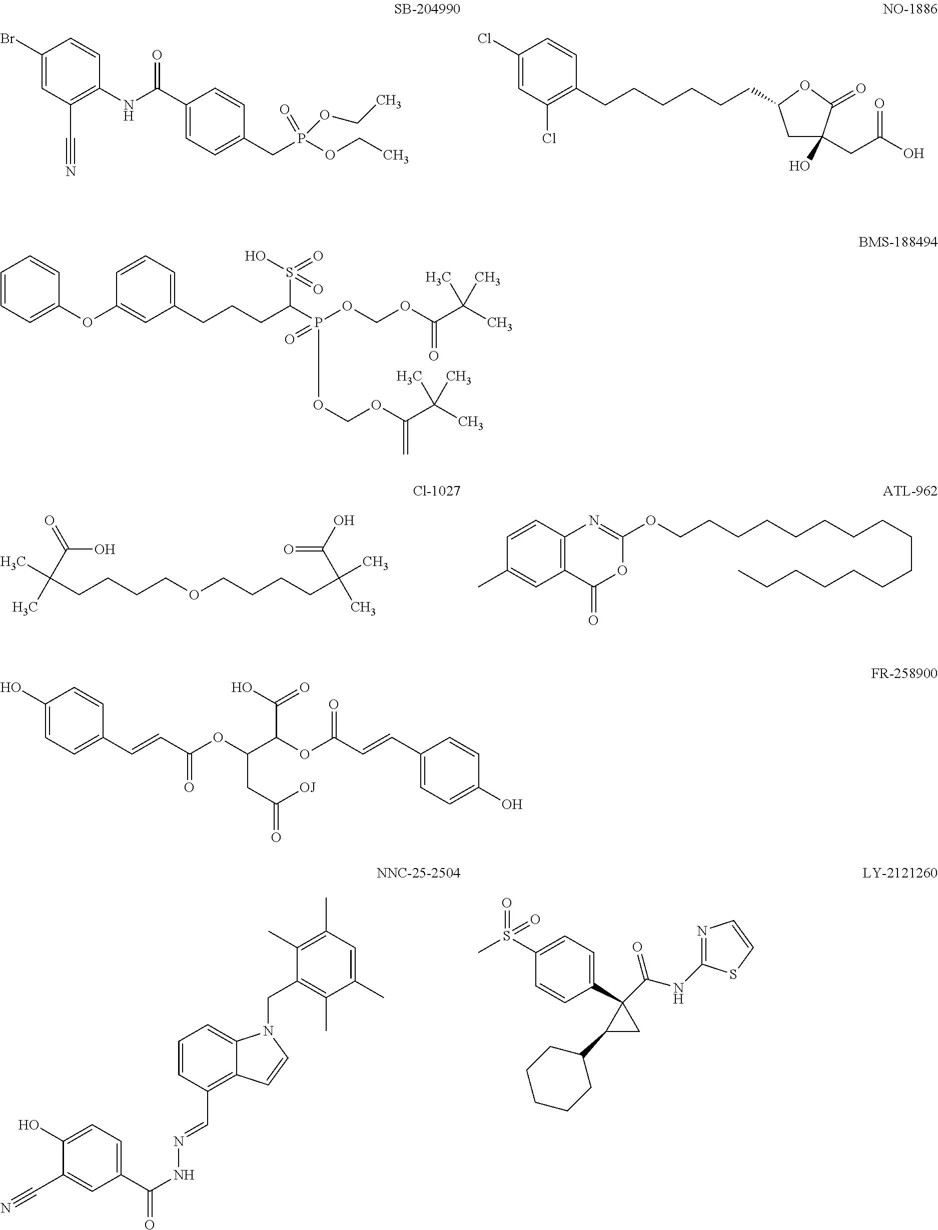

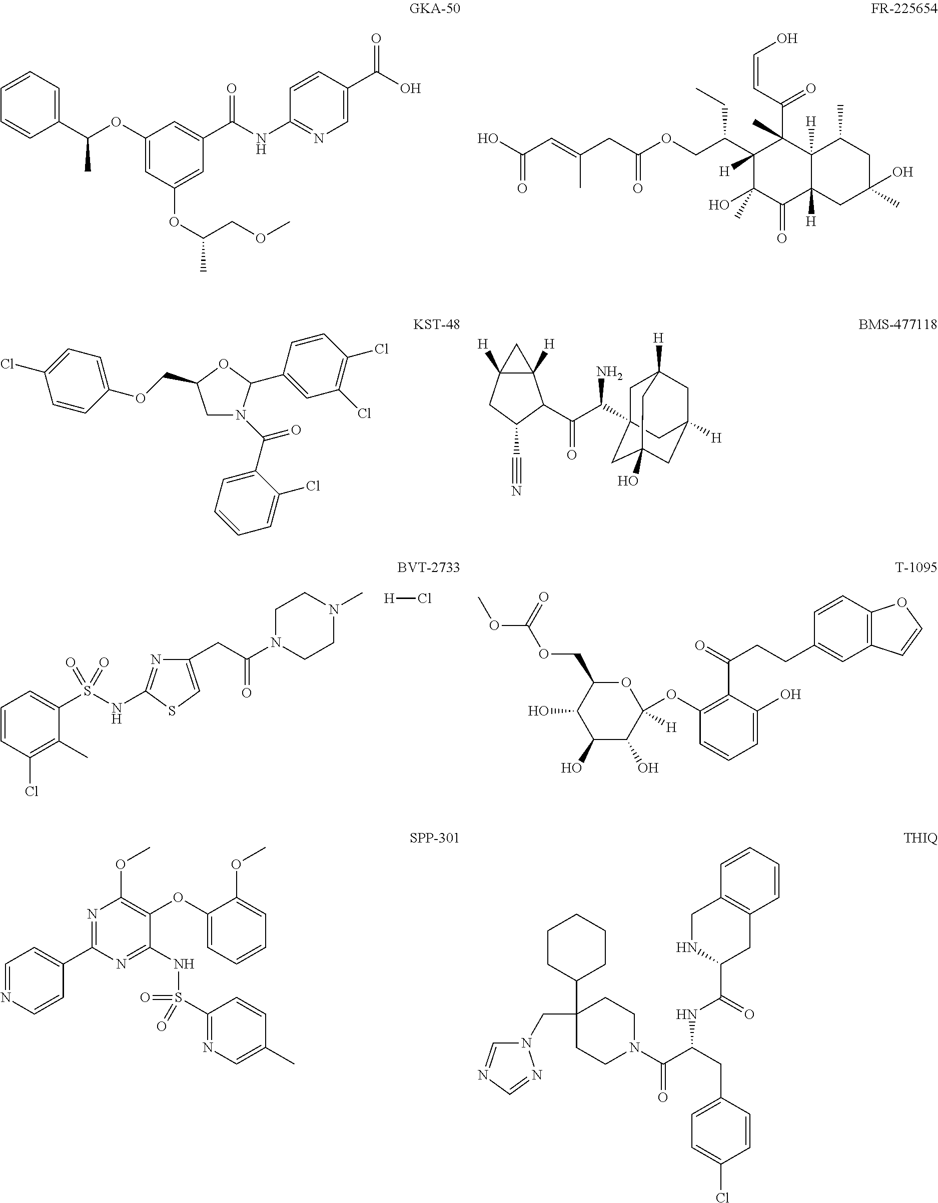

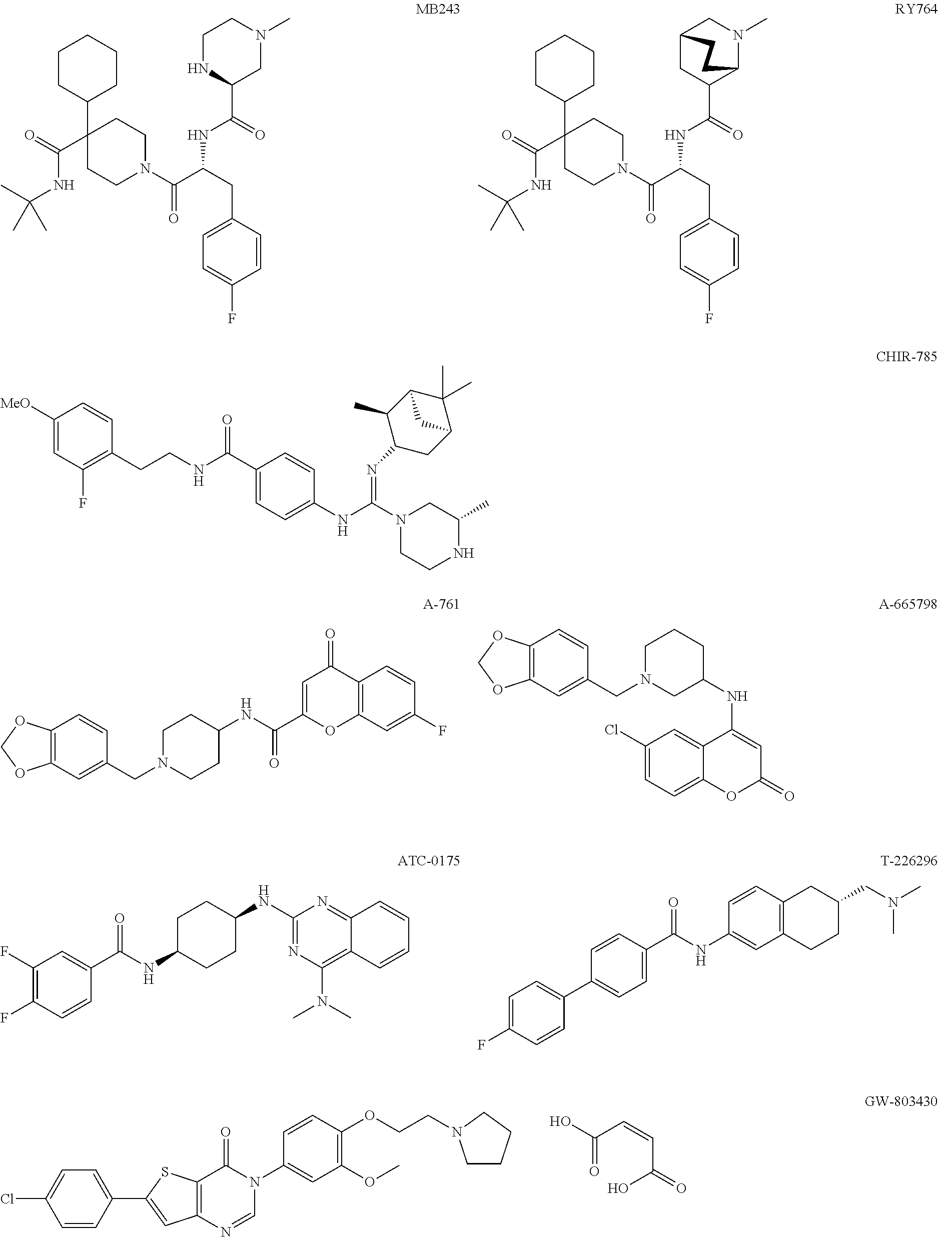

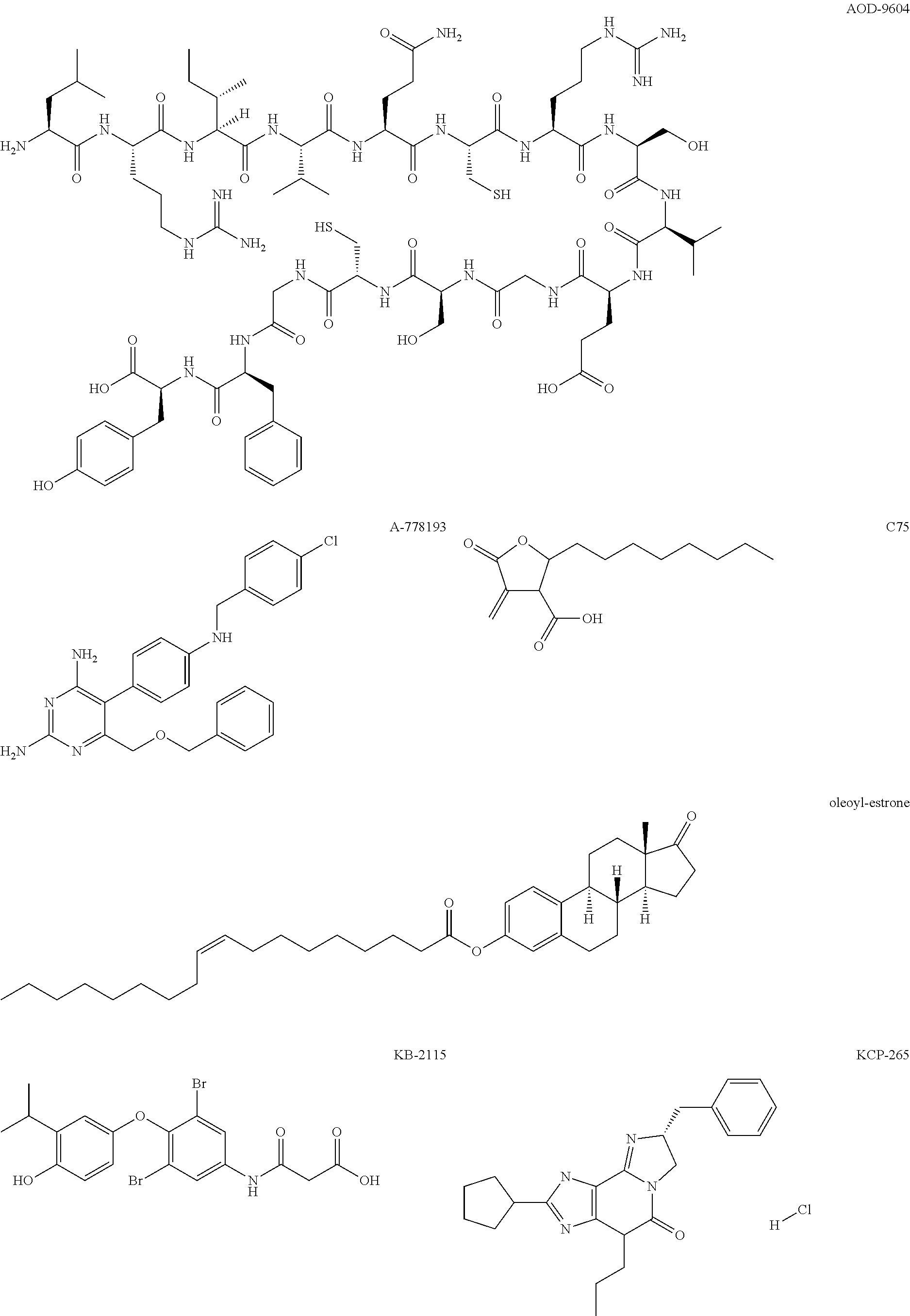

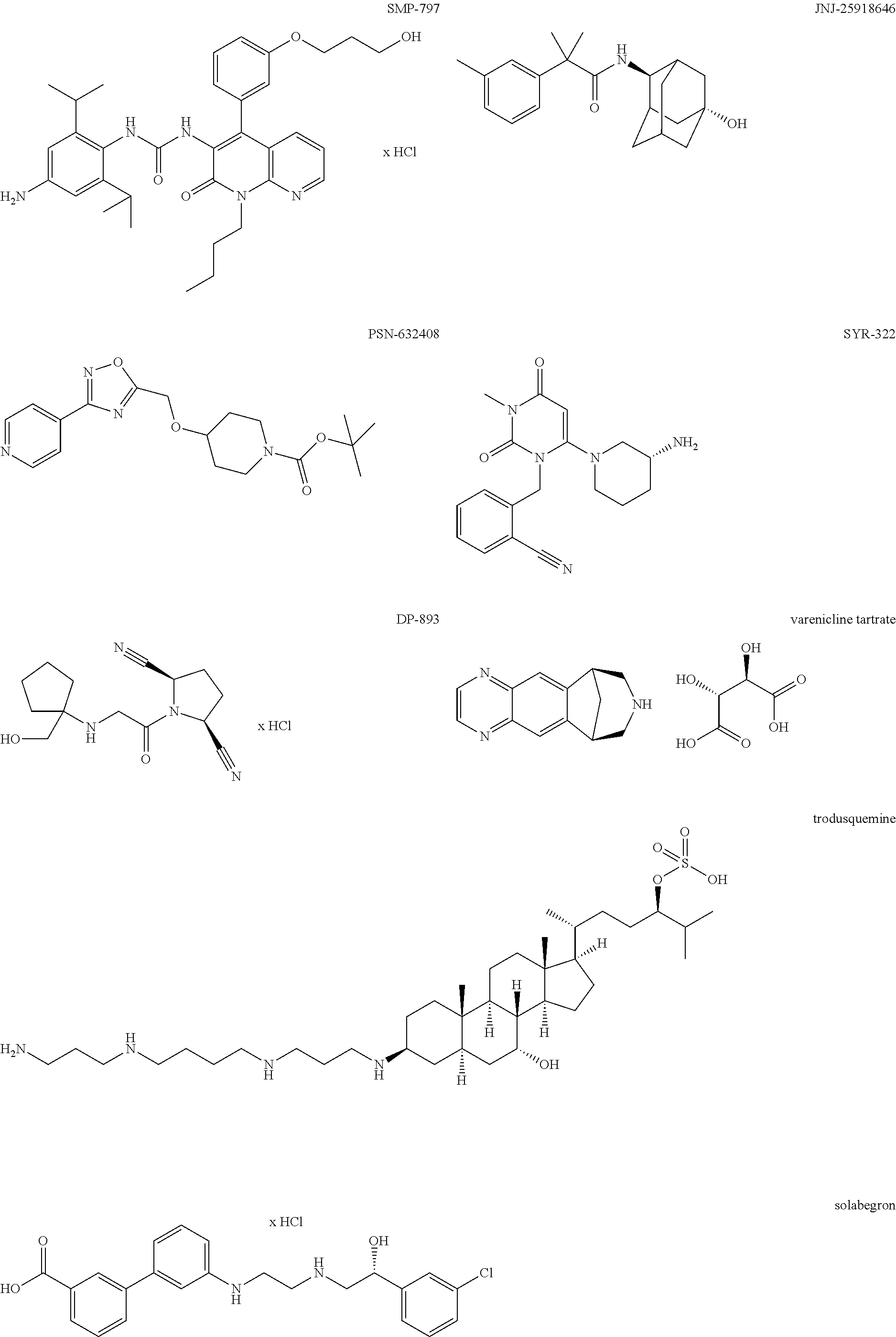

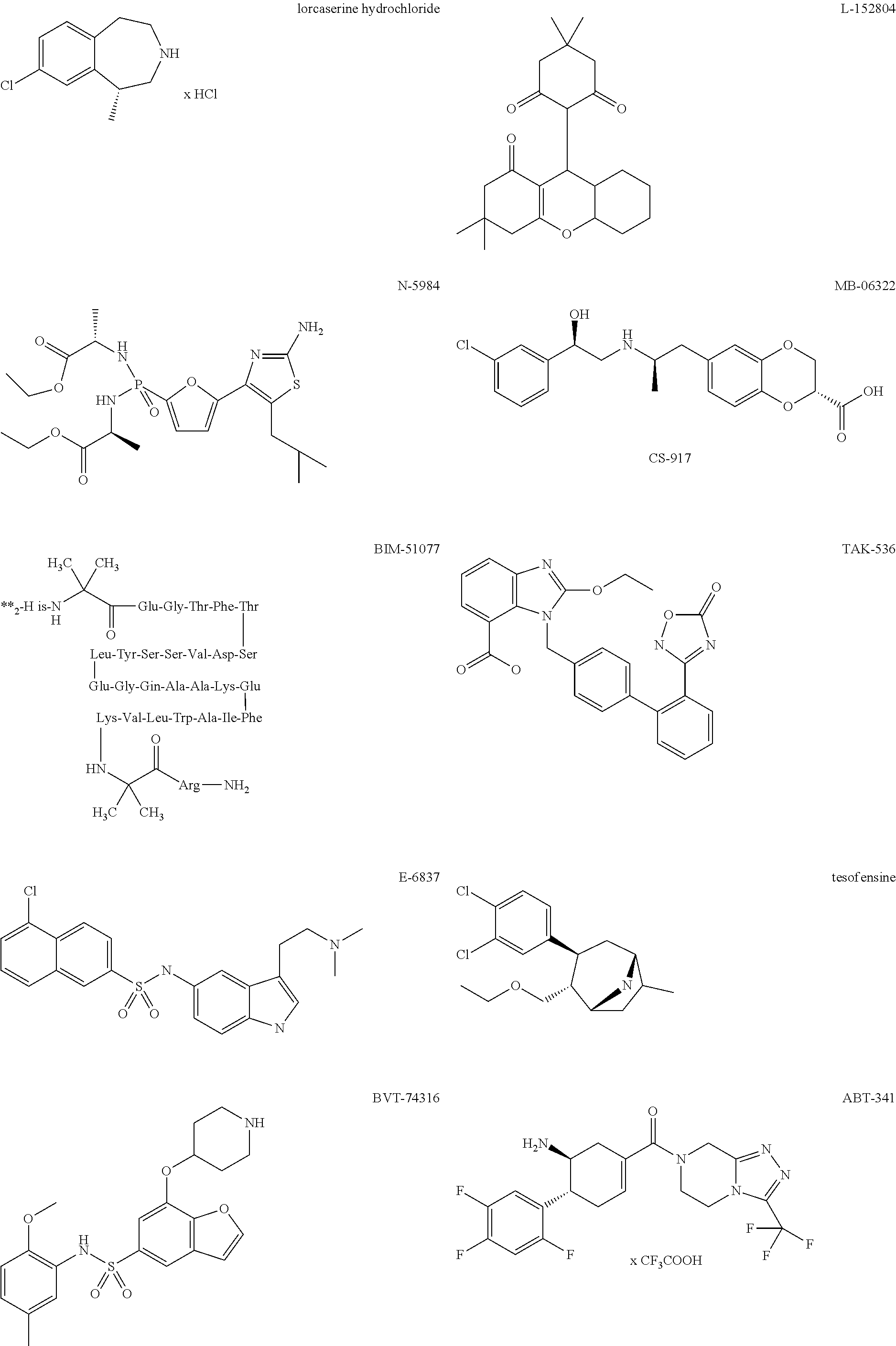

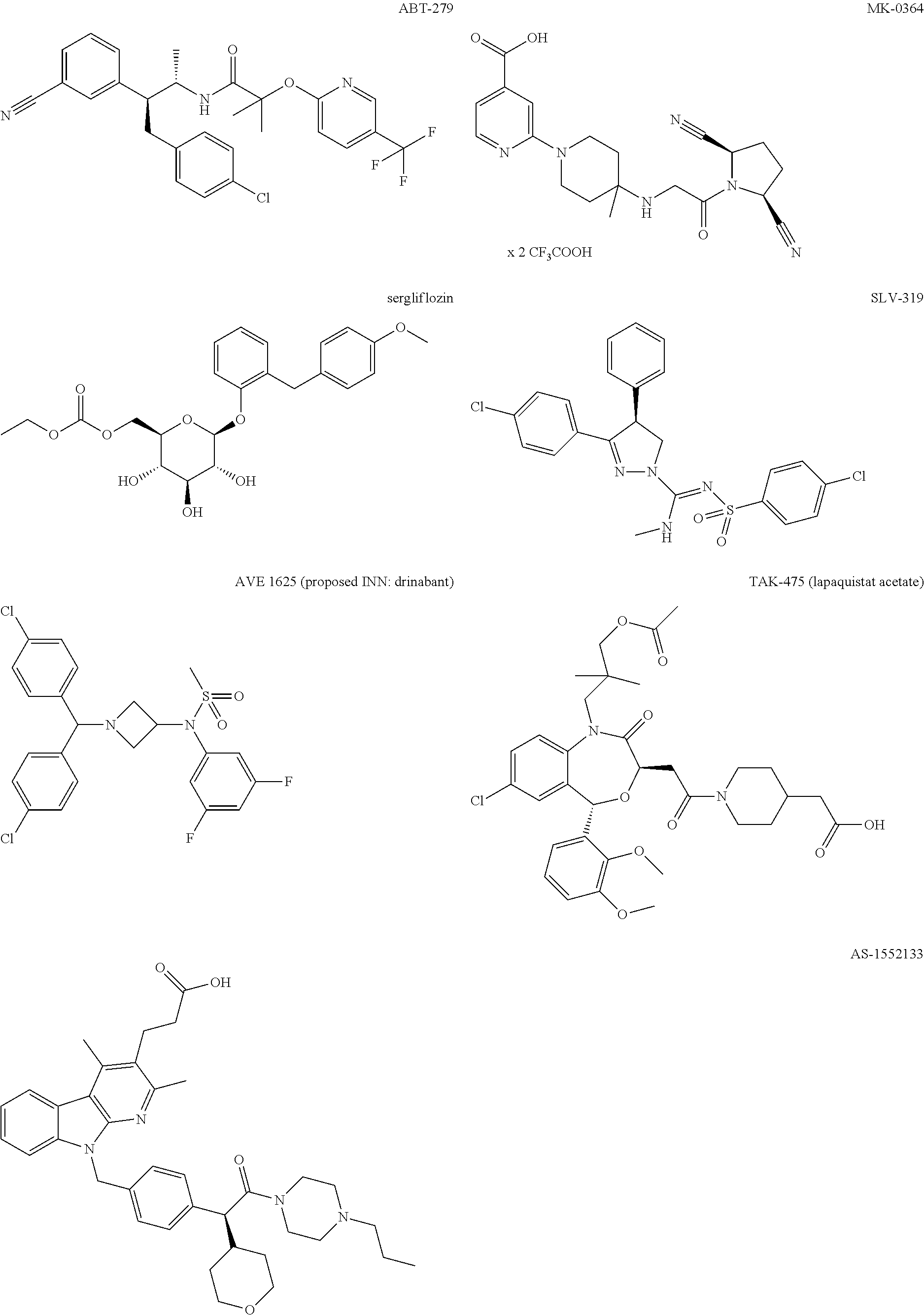

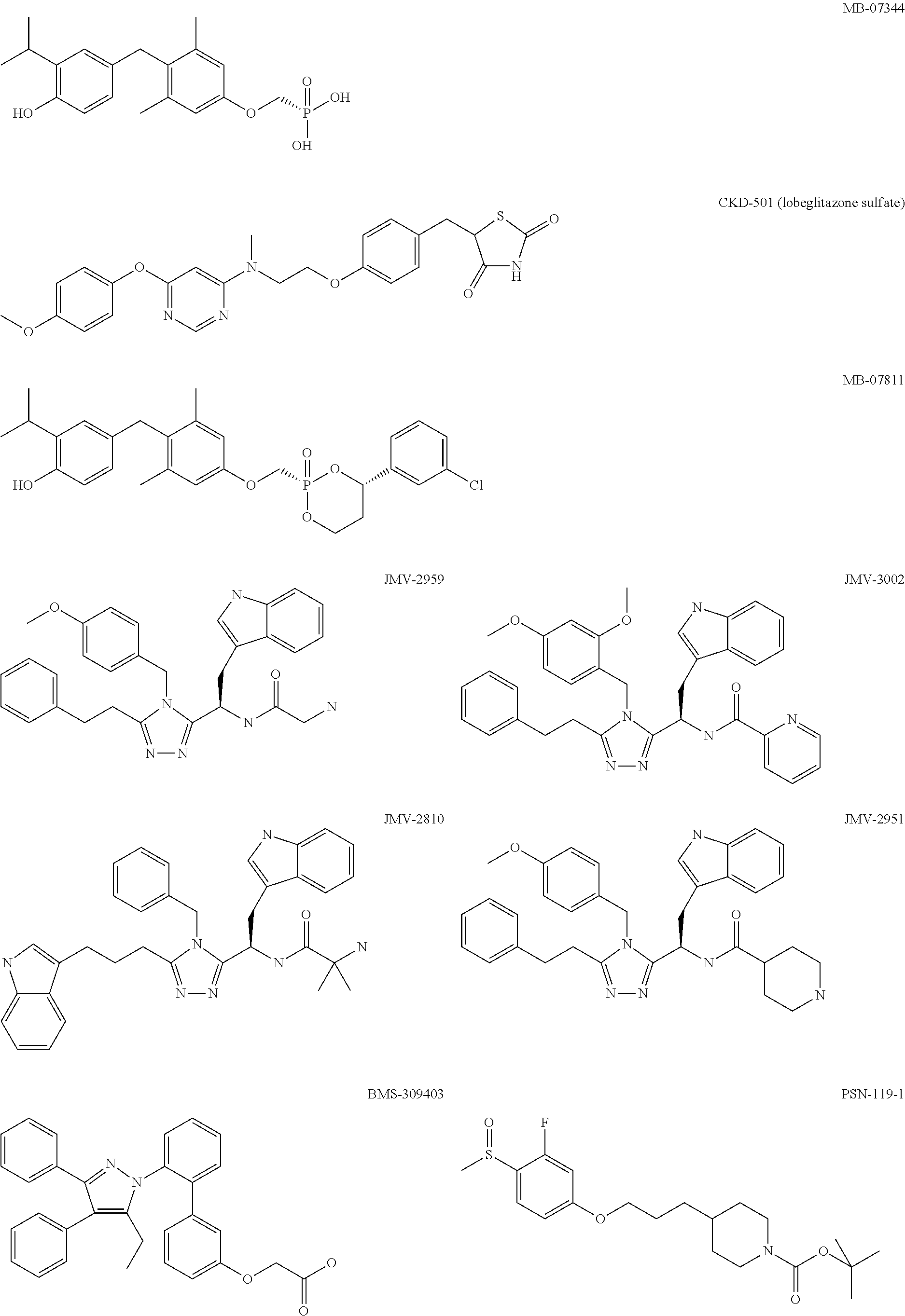

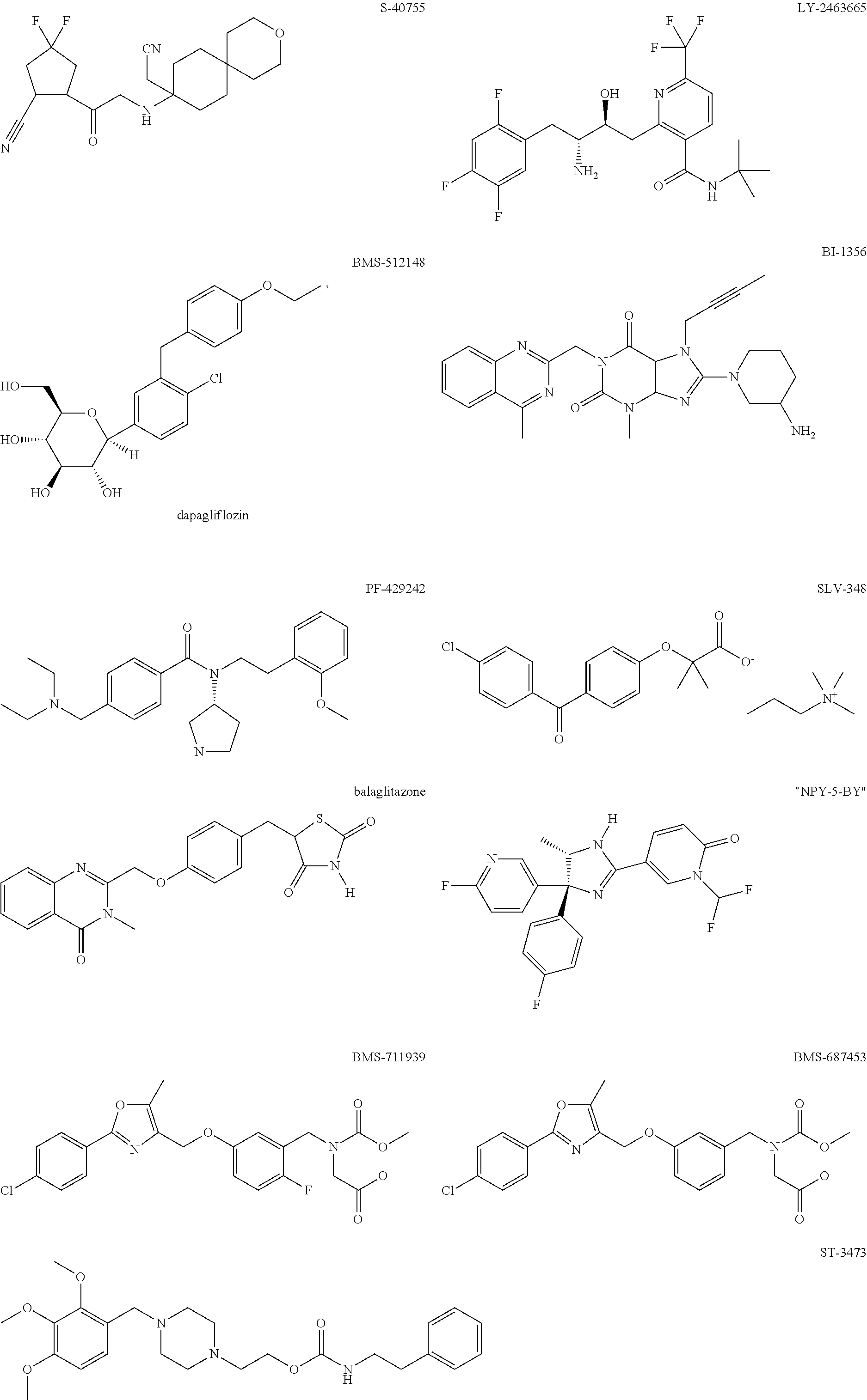

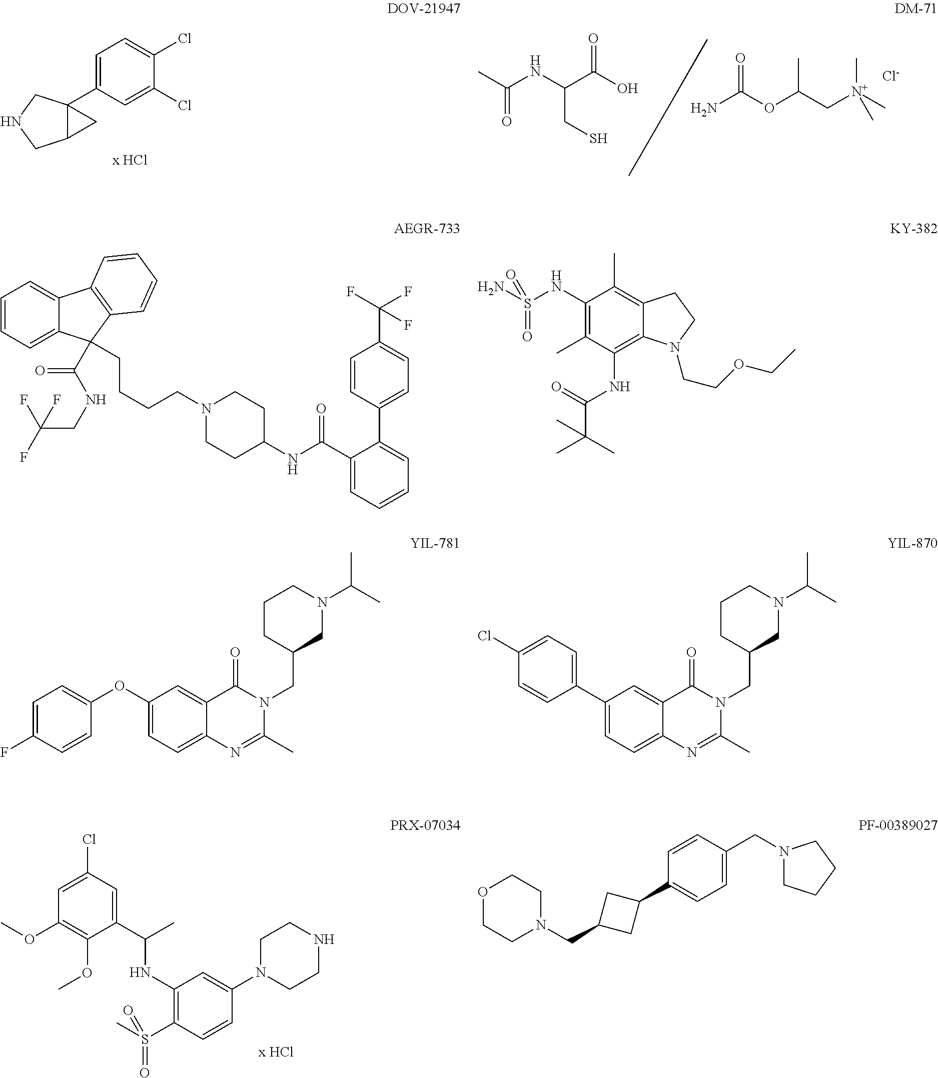

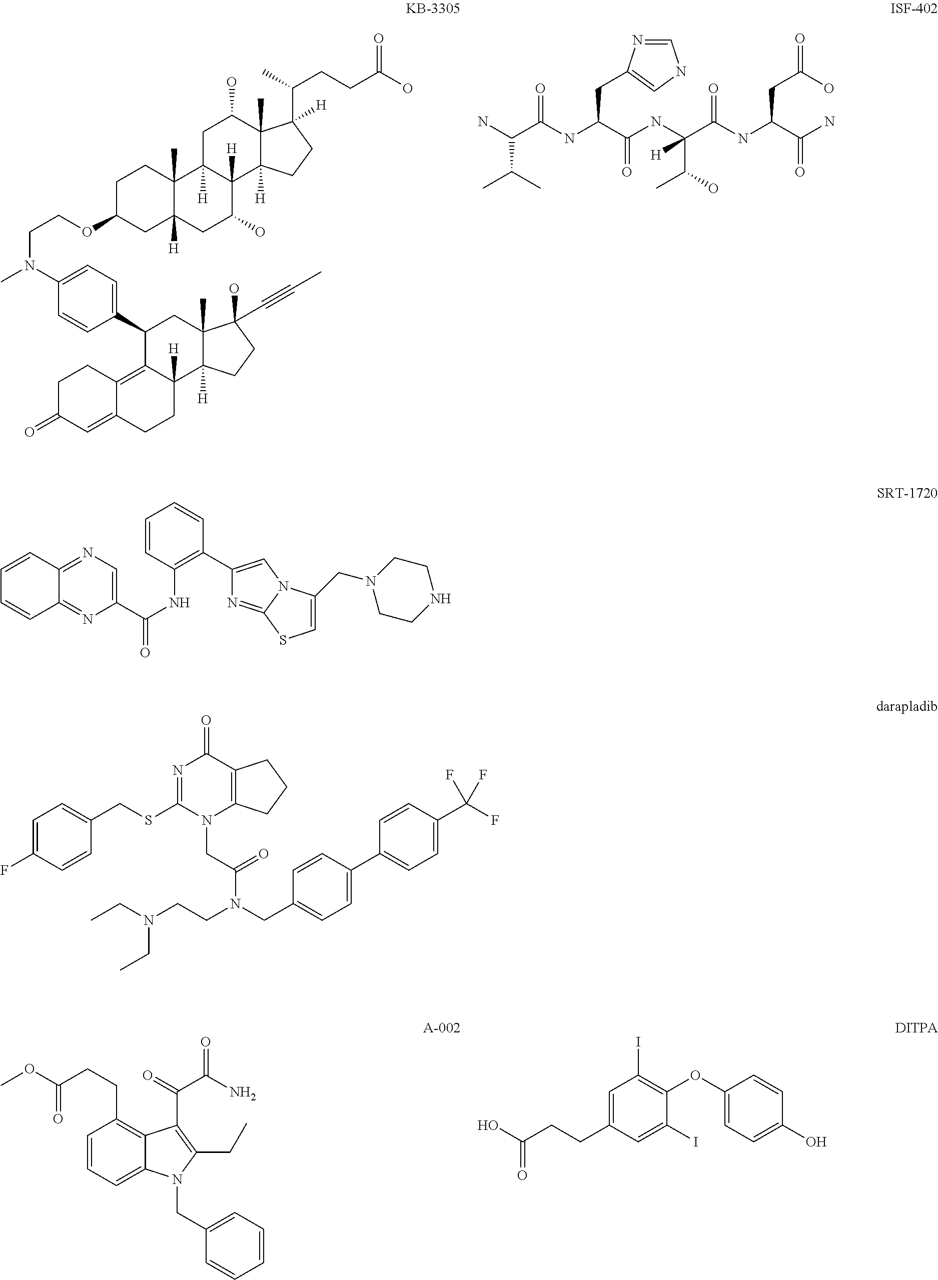

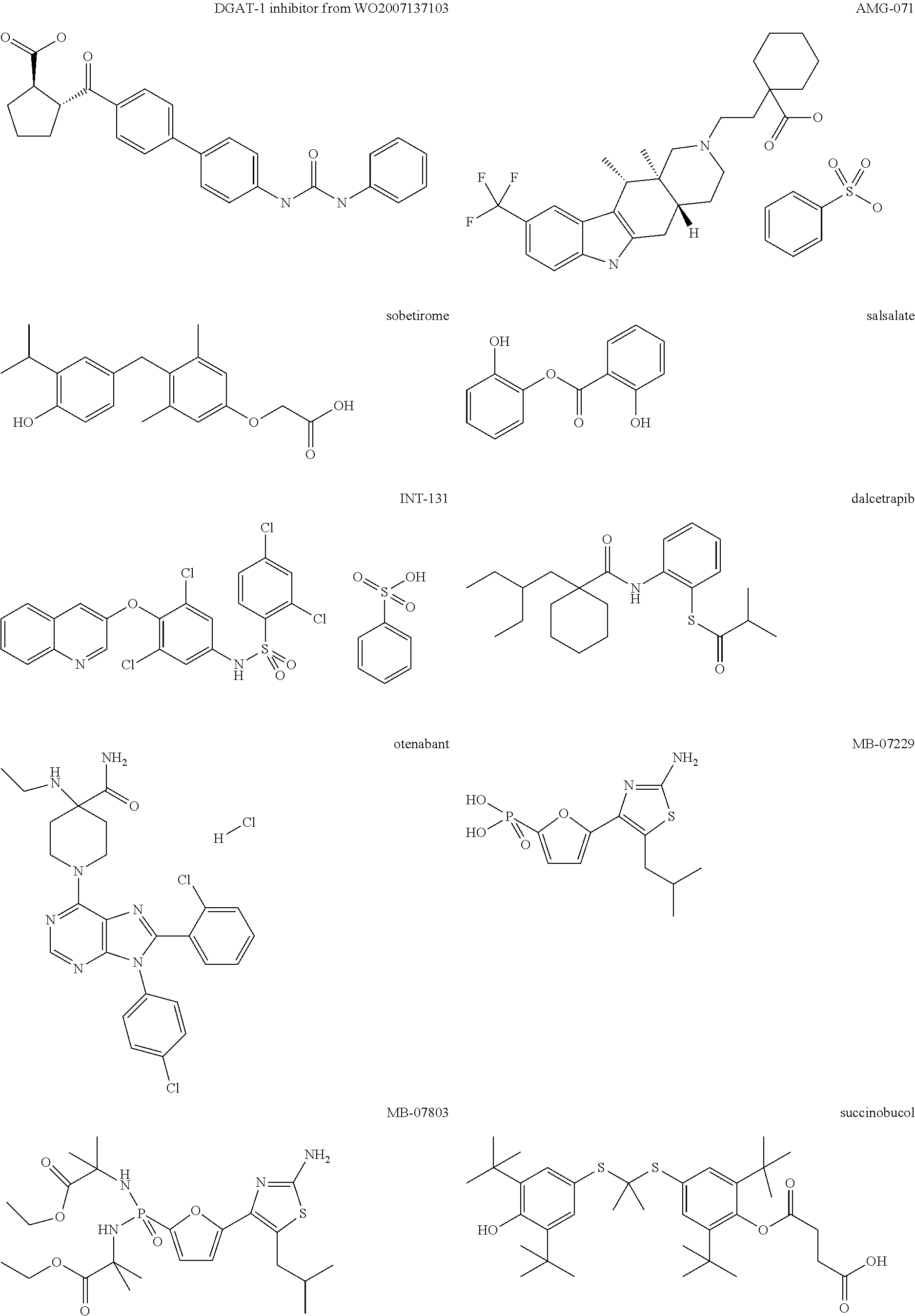

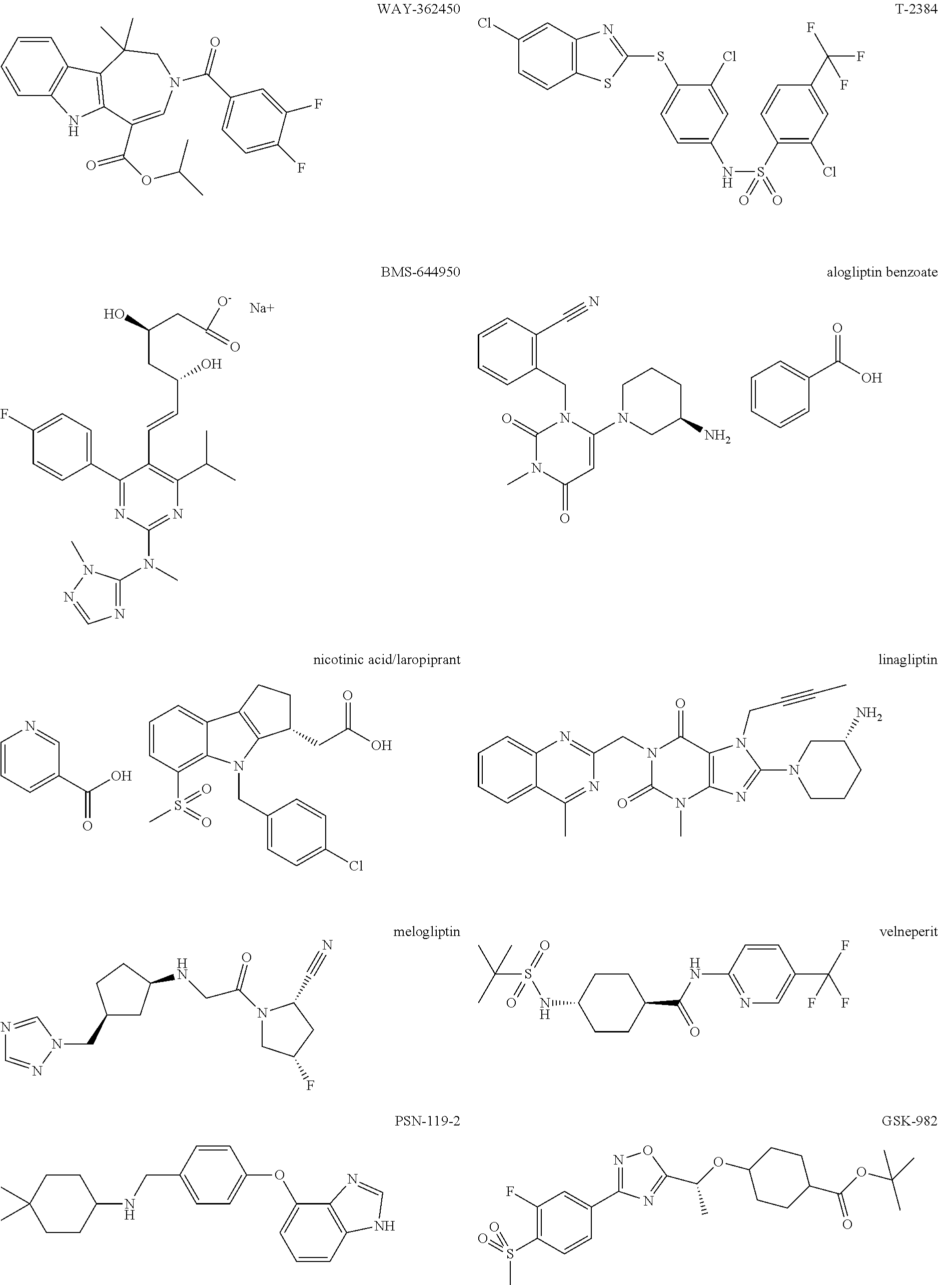

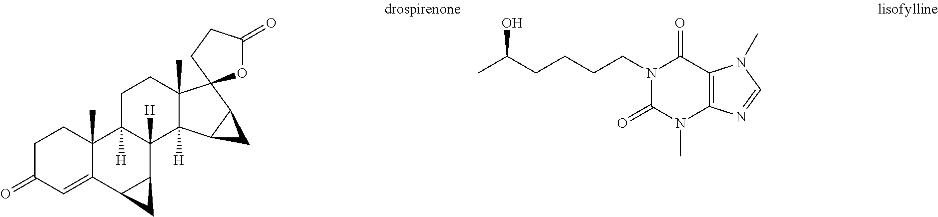

Also suitable are the following active ingredients for combination preparations:

all antiepileptics specified in the Rote Liste 2007, chapter 15;

all antihypertensives specified in the Rote Liste 2007, chapter 17;

all hypotonics specified in the Rote Liste 2007, chapter 19;

all anticoagulants specified in the Rote Liste 2007, chapter 20;

all arteriosclerosis drugs specified in the Rote Liste 2007, chapter 25;

all beta receptors, calcium channel blockers and inhibitors of the renin angiotensin system specified in the Rote Liste 2007, chapter 27;

all diuretics and perfusion-promoting drugs specified in the Rote Liste 2007, chapter 36 and 37;

all withdrawal drugs/drugs for the treatment of addictive disorders specified in the Rote Liste 2007, chapter 39;

all coronary drugs and gastrointestinal drugs specified in the Rote Liste 2007, chapter 55 and 60;

all migraine drugs, neuropathy preparations and Parkinson's drugs specified in the Rote Liste 2007, chapter 61, 66 and 70.

The invention further provides processes for preparing the compounds of the general formula I, wherein the compounds of the formula I are obtained in a procedure analogous to the reaction schemes which follow.

### Method “A”:

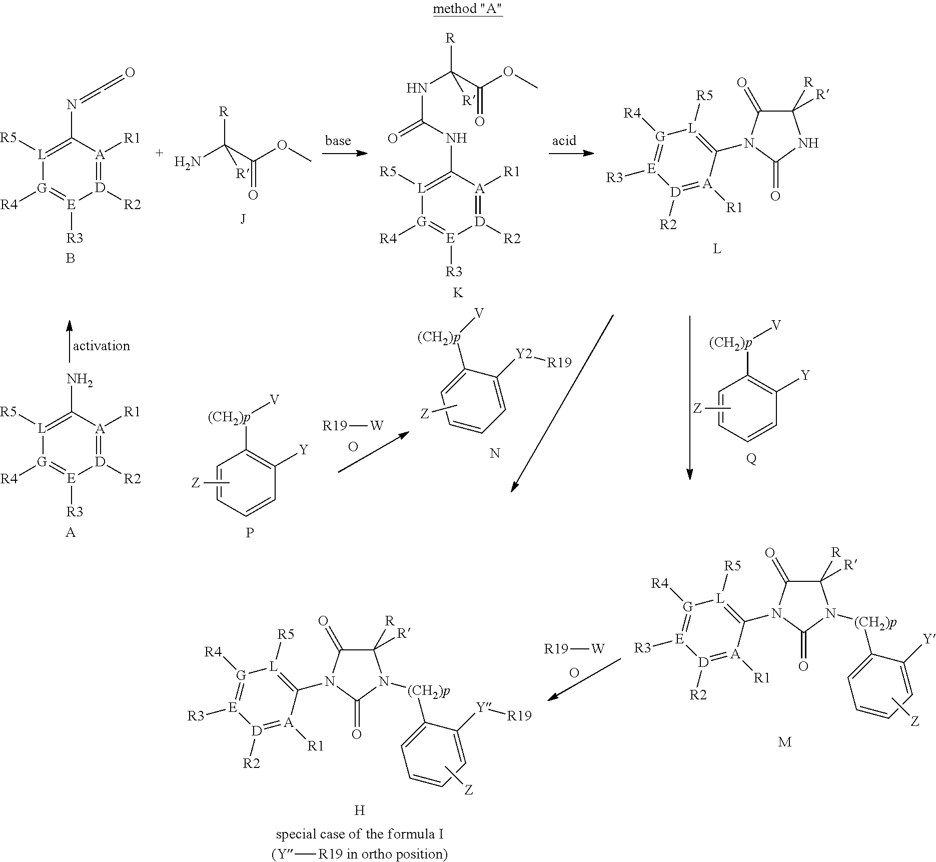

In a first method “A”, the procedure is to convert a suitably substituted aniline of the formula A in which the R1 to R5 radicals are in some circumstances present in protected form to an isocyanate of the formula B. This reaction can be carried out, for example, with phosgene in toluene or with diphosgene or triphosgene. The isocyanate B is subsequently reacted with the methyl ester or another ester (e.g. tert-butyl) of the amino acid J in which R and R′ are each as defined in formula I, or a salt of an ester of the amino acid J, with addition of base (e.g. triethylamine), to give a urea of the formula K. This urea can be ring-closed under basic or acidic conditions, preferably acidic conditions, to give the imidazolidine-2,4-dione of the formula L. The further conversion to a compound of the formula H, which constitutes the ortho-substituted special case of a compound of the formula I, can, for example, be effected by alkylating a suitably substituted compound Q where Z may be one or more substituents as described above in formula I, and Y is either a protected hydroxyl radical OR where R is, for example, acetyl, tert-butyl, benzyl or p-methoxybenzyl, or Y is a halogen radical, for example chlorine or bromine, and V is either a halogen atom, preferably a chlorine or bromine atom, or else, for example, an O—SO₂—C₆H₄-4-CH₃ radical or an O—SO₂—CH₃ radical or an O—SO₂—CF₃ radical, to obtain the compound M. After conversion by means of standard reactions of the Y radical defined as OR to a Y′ radical defined as OH, M can be reacted further by copper catalysis under Ullmann conditions (e.g.: R. Frian, D. Kikelj: Synthesis 2006, 2271-2285) with aryl or heteroaryl halides, preferably bromides, to give compounds of the formula H. W in R19-W of the formula O is defined, for example, as —Br. This reaction can alternatively also be carried out in such a way that the Y radical in the compound Q is a halogen atom (e.g. bromine or chlorine). This compound of the formula M can then be reacted in a next step with a compound of the formula R19-W in which W is defined as —OH, under the copper-catalyzed conditions outlined above, to give a compound of the formula H.

Instead of the copper-catalyzed Ullmann reaction, it is also possible to utilize the palladium-catalyzed diaryl ether coupling reaction (e.g.: A. Aranyos et al.: J. Am. Chem. Soc. 121 (1999) 4369-4378).

A further variant is that of intermolecular nucleophilic substitution (see, for example: F. Li et al.: Synthesis 2005, 1305; M. Chaouchi et al.: Eur. J. Org. Chem. 2002, 1278; S.-L. Cui et al.: Synlett 2004, 1829).

A further useful variant is that of the cross-coupling of phenols with aryl- or heteroarylboronic acids or trifluoroborates (see, for example: D. M. T. Chan et al.: Tetrahedron Lett. 39 (1998) 2933; D. M. T. Chan et al.: Tetrahedron Lett. 44 (2003) 3863; T. D. Quach et al.: Org. Lett. 5 (2003) 1381). This reaction can be conducted in such a way that either a compound of the formula M in which Y′ is OH is reacted with a compound of the formula O in which W is —B(OH)₂ or —BF₃⁻K⁺ to give a compound of the formula H in which Y″ is oxygen. Or a compound of the formula Min which Y′ is —B(OH)₂ or BF₃⁻J⁺ is reacted with a compound of the formula O in which W is hydroxyl.

In a corresponding manner, P can be reacted with O to give N, which is then reacted with L to give H.

Any protecting groups present in the compound H can be removed at the end.

The process described here can also be applied correspondingly to other chalcogen derivatives, for example diaryl thioethers.

The formula H shown here constitutes a special case of the formula I in which the Y″-R19 radical in formula I is in the ortho position; this radical may correspondingly also be in the meta or para position.

In another method “B”,

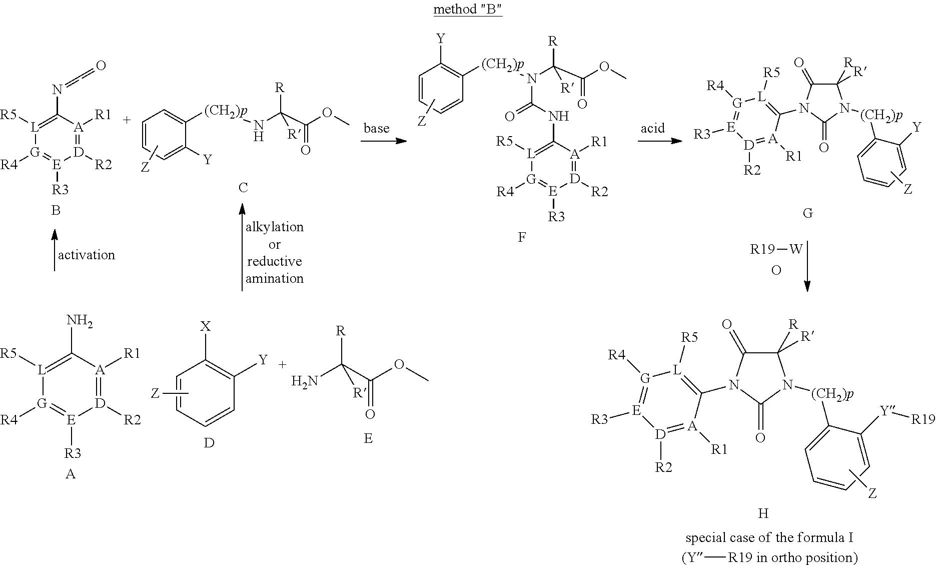

the isocyanate B is reacted with a suitably substituted amino acid ester derivative C in which the particular substituents may be provided with protecting groups, and where the methyl ester shown in the scheme is a nonlimiting example of an ester, and where Y is either a protected hydroxyl radical OR where R is, for example, acetyl, tert-butyl, benzyl or p-methoxybenzyl, or Y is a halogen radical, for example chlorine or bromine, with addition of a base (e.g. triethylamine), to give a urea of the formula F. The amino acid ester derivative C can be prepared from the compound D in which Z may be one or more substituents as described above in formula I, and where Y is either a protected hydroxyl radical OR where R is, for example, acetyl, tert-butyl, benzyl or p-methoxybenzyl, or Y is a halogen radical, for example chlorine or bromine, and X is a (CH₂)ₚ—U moiety in which U may be defined as Cl, Br, I, O—SO₂—C₆H₄-4-CH₃, O—SO₂—CH₃ or O—SO₂—CF₃, with an amino acid ester of the formula E in which R and R′ are each as defined in formula I, under alkylating conditions. Alternatively, the compound of the formula C can be obtained by reductive amination of the aldehyde D (Z and Y as described above and X═(CH₂)ₚ—CHO) with the amino acid derivative E. The urea F can be ring-closed under basic or acidic conditions, preferably acidic conditions, to give the imidazolidine-2,4-dione of the formula G. A protected hydroxyl radical OR where R is, for example, acetyl, tert-butyl, benzyl or p-methoxybenzyl, in the compounds of the formula G can be converted to an —OH function by standard reactions. According to whether Y in the compound of the formula G is —OH or halogen (e.g. Cl or Br), reaction with compounds of the formula O in which W is either boronic acid (boronic ester) or —OH can prepare compounds of the formula H. Otherwise, the procedures described under “method A” for the reaction of G with O to give H apply.

Any protecting groups present in the compound H can be removed at the end of the reaction sequence.

The process described here can also be applied correspondingly to other chalcogen derivatives, for example diaryl thioethers.

The formula H shown here constitutes a special case of the formula I in which the Y″-R19 radical in formula I is in the ortho position; this radical may correspondingly also be in the meta or para position.

In a further method (method “C”),

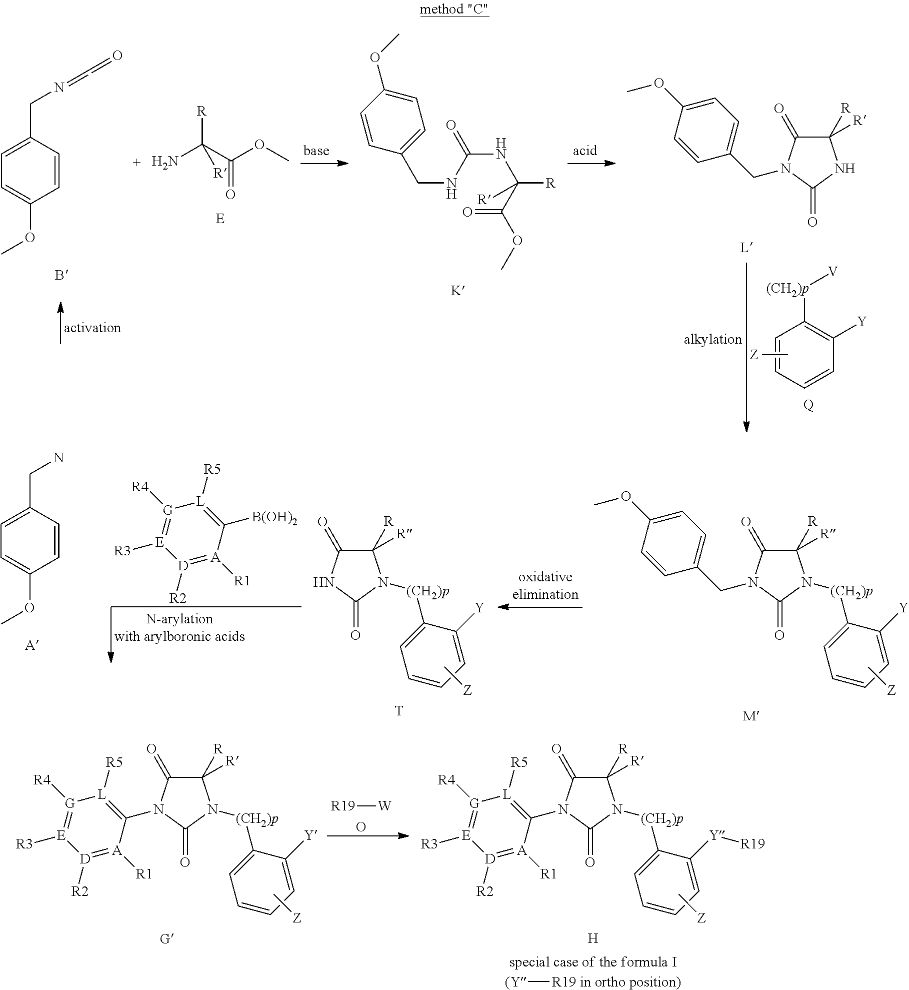

p-methoxybenzyl isocyanate B′ is reacted with an amino acid ester, for example E in which R and R′ are each as defined in formula I, under basic conditions, to give the urea K′. The urea K′ can be ring-closed under basic or acidic conditions, preferably acidic conditions, to give the imidazolidine-2,4-dione of the formula L′. The compounds M′ are obtained by reacting the compounds L′ with the compounds Q under alkylating conditions. Z, V and Y of the compounds Q are each defined as specified in method “A”. The p-methoxybenzyl group in the compounds M′ can be eliminated oxidatively to obtain the compounds T. The N-arylation of the imide nitrogen atom in compounds of the formula T using arylboronic acids of the formula S by processes as described, for example, in J.-B. Lan et al.: SYNLETT 2004, 1095-1097 or D. M. T. Chan et al.: Tetrahedron Lett. 1998, 39, 2933-2936, affords compounds of the formula G′. After conversion by means of standard reactions of the Y radical to a Y′ radical defined as —OH or obtaining the Y radical ═Y′=halogen, the compounds of the formula H can be obtained by reaction with compounds of the formula O as described above for method “A”.

Any protecting groups present in the compound H can be removed at the end. The process described here can also be applied correspondingly to other chalcogen derivatives, for example diaryl thioethers.

The formula H shown here constitutes a special case of the formula I in which the Y″-R19 radical in formula I is in the ortho position; this radical may correspondingly also be in the meta or para position.

The examples which follow serve to illustrate the invention in detail, without limiting it to the products and embodiments described in the examples.

### General Experimental Methods:

¹H NMR:

The ¹H NMR spectra were measured in deuterated dimethyl sulfoxide on a 500 MHz instrument (Bruker DRX 500) or on a 400 MHz instrument (Bruker DRX 400) at 300 K. Data: δ in ppm, multiplicity (s for singlet, d for doublet, t for triplet, q for quartet, m for multiplet, x H (number of hydrogen atoms))

HPLC-MS:

The HPLC-MS analyses were carried out on an LCT instrument from Waters. Column: YMC Jshere 33×2 4 μm; gradient [A]: (acetonitrile+0.05% trifluoroacetic acid):(water+0.05% trifluoroacetic acid) 5:95 (0 minutes) to 95:5 (3 minutes); gradient [B]: (acetonitrile+0.05% trifluoroacetic acid):(water+0.05% trifluoroacetic acid) 5:95 (0 minutes) to 95:5 (2.5 minutes) to 95:5 (3.0 minutes); gradient [C]: (acetonitrile+0.05% trifluoroacetic acid):(water+0.05% trifluoroacetic acid) 5:95 (0 minutes) to 95:5 (3.4 minutes) to 95:5 (4.4 minutes); detector: Tecan-LCT.

Chromatographic purification methods:

[RP1]: flow rate: 30 ml/min; gradient: acetonitrile/water+0.1% trifluoroacetic acid; 30 min. column: XTerra C18 5 μm 30×100 mm; detection: MS (ESI), UV (DAD).

[RP2]: flow rate: 150 ml/min; gradient: acetonitrile/water+0.1% trifluoroacetic acid; 20 min. column: XTerra C18 10 μm 50×250 mm; detection: MS (ESI), UV (DAD).

### EXAMPLE 1

### 4-[4,4-Dimethyl-2,5-dioxo-3-(2-phenoxybenzyl)-imidazolidin-1-yl]-benzonitrile

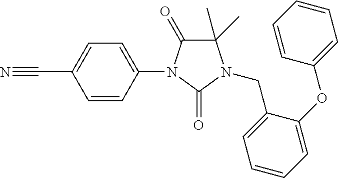

1) Preparation of 1-bromomethyl-2-phenoxybenzene (1.2):

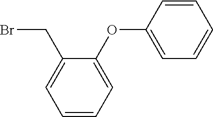

2.5 g (12.5 mmol) of 2-phenoxybenzyl alcohol were dissolved in 45 ml of dichloromethane and admixed dropwise at 5° C. with a solution of 1.35 g (5 mmol) of phosphorus tribromide in 5 ml of dichloromethane. The mixture stood at room temperature overnight. Thereafter, the reaction mixture was admixed with 5 ml of saturated sodium carbonate solution, and the organic phase was removed, dried over magnesium sulfate, filtered and concentrated under reduced pressure. This afforded 3.25 g (quantitative) of 1-bromomethyl-2-phenoxybenzene 1.2. ¹H NMR: 7.56, d, 1H, 7.4, m, 2H, 7.3, m, 1H, 7.15, m, 2H, 7.01, d, 2H, 6.81, d, 1H, 4.7, s, 2H. Molecular weight 261.99 (C₁₃H₁ₗBrO).

2) Preparation of tert-butyl 2-methyl-2-(2-phenoxybenzylamino)propionate (1.1):

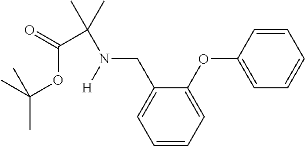

Compound 1.1 can be prepared by method “B”. To this end, 3.21 g (76.7 mmol) of lithium hydroxide hydrate were initially charged in 125 ml of dry dimethylformamide, admixed with 20 g of 4 A molecular sieve and stirred at room temperature for 30 minutes. Thereafter, 7.5 g (38.3 mmol) of tert-butyl 2-amino-2-methylpropionate hydrochloride were added and the mixture was stirred at room temperature for 15 minutes, before 11.09 g (42.16 mmol) of the bromide 1.2, dissolved in 25 ml of dry dimethylformamide, were added dropwise at room temperature. The reaction mixture was stirred at room temperature for 20 h. The reaction mixture was admixed with water and ethyl acetate, and the organic phase was removed, dried over magnesium sulfate, filtered and concentrated under reduced pressure. The residue was purified by chromatography (silica gel; 10/1 n-heptane/ethyl acetate) and afforded 8.3 g (64% yield) of tert-butyl 2-methyl-2-(2-phenoxybenzylamino)propionate 1.1. Molecular weight 341.19 (C₂₁H₂₇NO₃); retention time Rₜ=1.58 min. [B]; MS (ESI): 342.49 (MH⁺).

3) Preparation of 4-[4,4-dimethyl-2,5-dioxo-3-(2-phenoxybenzyl)imidazolidin-1-yl]-benzonitrile (1):

To 0.15 mmol of the amino acid ester 1.1 in 2 ml of dry acetonitrile was added 0.165 mmol of 4-isocyanatobenzonitrile. The mixture was stirred with exclusion of moisture at room temperature overnight. Thereafter, 0.1 ml of concentrated hydrochloric acid was added, and the mixture was stirred at room temperature for a further 3 h until completion of conversion (ring closure). The solvent was removed under reduced pressure; the residue was dissolved in 2 ml of dimethylformamide, filtered through a syringe filter and purified by means of preparative HPLC. This afforded the compound of example 1. ¹H NMR: 8.0, d, 2H, 7.7, d, 2H, 7.58, d, 1H, 7.4, m, 2H, 7.3, t, 1H, 7.16, m, 2H, 7.0, d, 2H, 6.9, d, 1H, 4.6, s, 2H, 1.4, s, 6H. Molecular weight 411.15 (C₂₅H₂₁N₃O₃); retention time Rₜ=1.92 min. [B]; MS (ESI): 412.16 (MH⁺).

The compounds of examples 2-74 and 84 (see table 1) were prepared in an analogous manner, by reacting compound 1.1 with the appropriate isocyanates.

For obtaining the compound of example 2 1-ethyl-3-isocyanatobenzene was used,

for 3 (4-isocyanatophenyl)acetonitrile,

for 4 1-(4-isocyanatophenyl)ethanone,

for 5 1-(3-isocyanatophenyl)ethanone,

for 6 1-isocyanato-3-methylsulfanylbenzene,

for 7 methyl 3-isocyanatobenzoate,

for 8 1-isocyanato-3-trifluoromethylbenzene,

for 9 3-isocyanato-5-methyl-2-trifluoromethylfuran,

for 10 ethyl 4-isocyanatobenzoate,

for 11 2-fluoro-1-isocyanato-3-trifluoromethylbenzene,

for 12 1-fluoro-2-isocyanato-4-trifluoromethylbenzene,

for 13 1-benzyl-4-isocyanatobenzene,

for 14 1-isocyanato-3-trifluoromethylsulfanylbenzene,

for 15 3-isocyanatopyridine,

for 16 3-isocyanatobenzonitrile,

for 17 1-isocyanato-3-phenoxybenzene,

for 18 1-isocyanato-4-phenoxybenzene,

for 19 (4-isocyanatophenyl)phenylmethanone,

for 20 phenyl isocyanate,

for 21 2-isocyanatothiophene,

for 22 1-isocyanato-2-methylbenzene,

for 23 1-isocyanato-4-methylbenzene,

for 24 1-fluoro-2-isocyanatobenzene,

for 25 1-fluoro-3-isocyanatobenzene,

for 26 1-fluoro-4-isocyanatobenzene,

for 27 2-isocyanatobenzonitrile,

for 28 1-isocyanato-2,4-dimethylbenzene,

for 29 1-isocyanato-3,5-dimethylbenzene,

for 30 4-isocyanato-1,2-dimethylbenzene,

for 31 1-ethyl-4-isocyanatobenzene,

for 32 1-isocyanato-3-methoxybenzene,

for 33 1-isocyanato-2-methoxybenzene,

for 34 1-isocyanato-4-methoxybenzene,

for 35 2-fluoro-4-isocyanato-1-methylbenzene,

for 36 1-chloro-3-isocyanatobenzene,

for 37 1-chloro-4-isocyanatobenzene,

for 38 1-chloro-2-isocyanatobenzene,

for 39 1,4-difluoro-2-isocyanatobenzene,

for 40 1,3-difluoro-5-isocyanatobenzene,

for 41 1-isocyanato-4-isopropylbenzene,

for 42 1-isocyanato-2,4,6-trimethylbenzene,

for 43 1-isocyanato-2,4,5-trimethylbenzene,

for 44 1-ethoxy-4-isocyanatobenzene,

for 45 1-isocyanato-2-methylsulfanylbenzene,

for 46 2-chloro-4-isocyanato-1-methylbenzene,

for 47 4-chloro-1-isocyanato-2-methylbenzene,

for 48 1-tert-butyl-4-isocyanatobenzene,

for 49 1-isocyanato-2,4-dimethoxybenzene,

for 50 1-isocyanato-3,5-dimethoxybenzene,

for 51 4-isocyanato-1,2-dimethoxybenzene,

for 52 2-chloro-4-isocyanato-1-methoxybenzene,

for 53 1-isocyanato-2-trifluoromethylbenzene,

for 54 1-isocyanato-4-trifluoromethylbenzene,

for 55 1,3-dichloro-5-isocyanatobenzene,

for 56 2,4-dichloro-1-isocyanatobenzene,

for 57 1,2-dichloro-3-isocyanatobenzene,

for 58 1,4-dichloro-2-isocyanatobenzene,

for 59 2,6-dichloro-4-isocyanatopyridine,

for 60 ethyl 3-isocyanatobenzoate,

for 61 2-isocyanatobiphenyl,

for 62 4-isocyanatobiphenyl,

for 63 1-isocyanato-4-trifluoromethoxybenzene,

for 64 1-benzyl-3-isocyanatobenzene,

for 65 1-benzyl-2-isocyanatobenzene,

for 66 1-isocyanato-2-phenoxybenzene,

for 67 2-chloro-1-isocyanato-5-trifluoromethylbenzene,

for 68 4-chloro-1-isocyanato-2-trifluoromethylbenzene,

for 69 1-benzyloxy-4-isocyanatobenzene,

for 70 1-heptyloxy-4-isocyanatobenzene,

for 71 4-chloro-2-isocyanato-1-phenoxybenzene,

for 72 1-isocyanato-3,5-bis(trifluoromethyl)benzene,

for 73 2-(4-isocyanatophenyl)-6-methylbenzothiazole,

for 74 2,6-difluoro-1-isocyanatobenzene, and

for 84 5-isocyanatoindane.

TABLE 1

| | | | | | | Reten- | |
|---|---|---|---|---|---|---|---|
| | | | | | | tion | HPLC/ |
| Exam- | | | Molec- | | | time | MS |
| ple | | Empirical | ular | | | T_{R} | meth- |
| No. | Product | formula | weight | MH⁺ | ¹H NMR | [min.] | od |
| 2 | 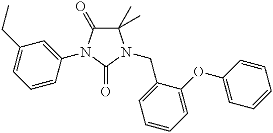 | C₂₆H₂₆N₂O₃ | 414.19 | 415.20 | | 2.11 | B |
| 3 | 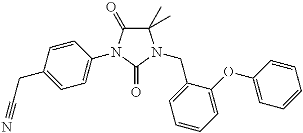 | C₂₆H₂₃N₃O₃ | 425.17 | 426.16 | | 1.83 | B |
| 4 | 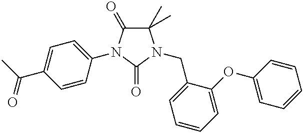 | C₂₆H₂₄N₂O₄ | 428.17 | 429.17 | | 2.07 | B |
| 5 | 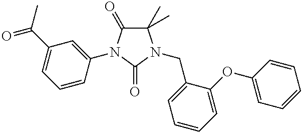 | C₂₆H₂₄N₂O₄ | 428.17 | 429.17 | | 2.06 | B |
| 6 | 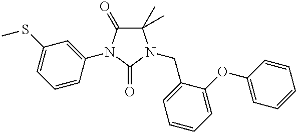 | C₂₅H₂₄N₂O₃S | 432.15 | 433.14 | | 2.24 | B |
| 7 | 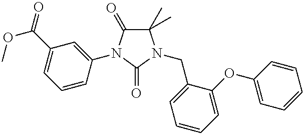 | C₂₆H₂₄N₂O₅ | 444.17 | 445.16 | | 2.15 | B |
| 8 | 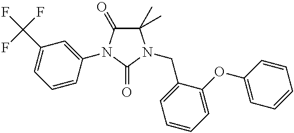 | C₂₅H₂₁F₃N₂O₃ | 454.15 | 455.15 | | 2.34 | B |
| 9 | 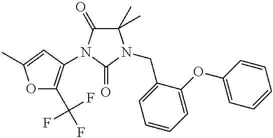 | C₂₄H₂₁F₃N₂O₄ | 458.15 | 459.14 | | 2.31 | B |
| 10 | 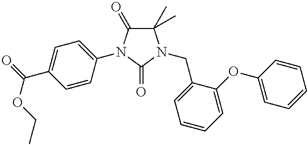 | C₂₇H₂₆N₂O₅ | 458.18 | 459.17 | | 2.28 | B |
| 11 | 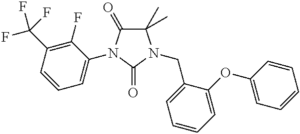 | C₂₅H₂₀F₄N₂O₃ | 472.14 | 473.14 | | 2.33 | B |
| 12 | 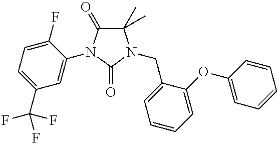 | C₂₅H₂₀F₄N₂O₃ | 472.14 | 473.14 | | 2.32 | B |
| 13 | 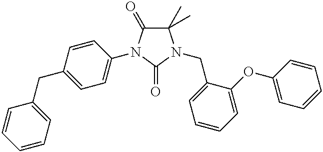 | C₃₁H₂₈N₂O₃ | 476.21 | 477.17 | | 2.45 | B |
| 14 | 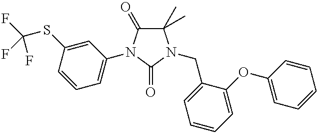 | C₂₅H₂₁F₃N₂O₃S | 486.12 | 487.12 | | 2.45 | B |
| 15 | 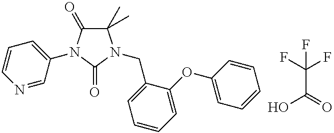 | C₂₃H₂₁N₃O₃•C₂HF₃O₂ | 387.16 | 388.17 (free base) | | 1.98 | C |
| 16 | 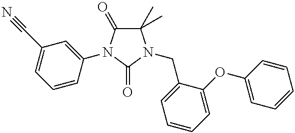 | C₂₅H₂₁N₃O₃ | 411.16 | 412.22 | | 2.62 | C |
| 17 | 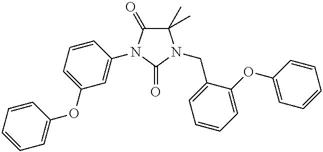 | C₃₀H₂₆N₂O₄ | 478.19 | 479.25 | | 3.05 | C |
| 18 | 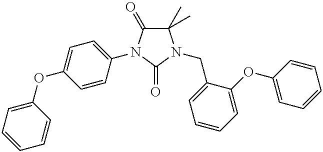 | C₃₀H₂₆N₂O₄ | 478.19 | 479.25 | | 3.10 | C |
| 19 | 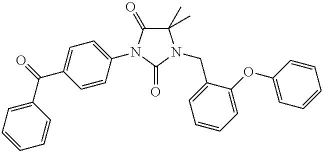 | C₃₁H₂₆N₂O₄ | 490.19 | 491.25 | | 2.93 | C |
| 20 | 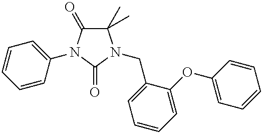 | C₂₄H₂₂N₂O₃ | 386.16 | 387.22 | | 2.62 | C |
| 21 | 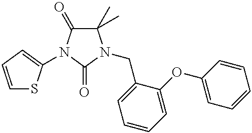 | C₂₂H₂₀N₂O₃S | 392.12 | 393.19 | | 2.71 | C |
| 22 | 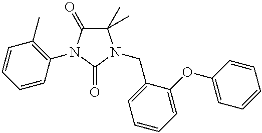 | C₂₅H₂₄N₂O₃ | 400.18 | 401.24 | | 2.65 | C |
| 23 | 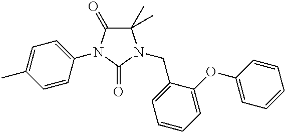 | C₂₅H₂₄N₂O₃ | 400.18 | 401.23 | | 2.80 | C |
| 24 | 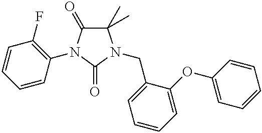 | C₂₄H₂₁FN₂O₃ | 404.15 | 405.20 | | 2.63 | C |
| 25 | 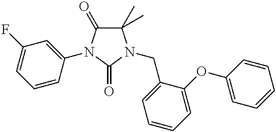 | C₂₄H₂₁FN₂O₃ | 404.15 | 405.21 | | 2.72 | C |
| 26 | 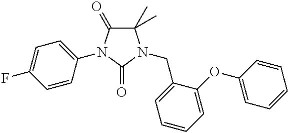 | C₂₄H₂₁FN₂O₃ | 404.15 | 405.21 | | 2.66 | C |
| 27 | 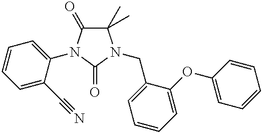 | C₂₅H₂₁N₃O₃ | 411.16 | 412.19 | | 2.53 | C |
| 28 | 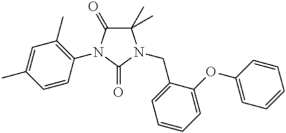 | C₂₆H₂₆N₂O₃ | 414.19 | 415.23 | | 2.80 | C |
| 29 | 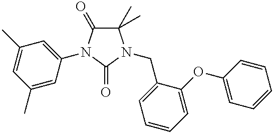 | C₂₆H₂₆N₂O₃ | 414.19 | 415.23 | | 2.93 | C |
| 30 | 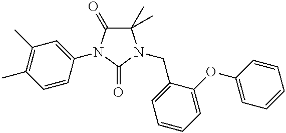 | C₂₆H₂₆N₂O₃ | 414.19 | 415.24 | | 2.85 | C |
| 31 | 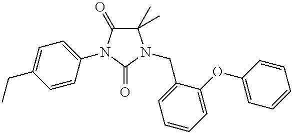 | C₂₆H₂₆N₂O₃ | 414.19 | 415.24 | | 2.93 | C |
| 32 | 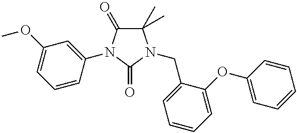 | C₂₅H₂₄N₂O₄ | 416.17 | 417.21 | | 2.65 | C |
| 33 | 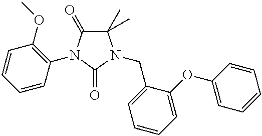 | C₂₅H₂₄N₂O₄ | 416.17 | 417.19 | | 2.55 | C |
| 34 | 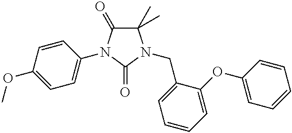 | C₂₅H₂₄N₂O₄ | 416.17 | 417.20 | | 2.62 | C |
| 35 | 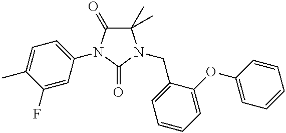 | C₂₅H₂₃FN₂O₃ | 418.17 | 419.44 | | 2.62 | B |
| 36 | 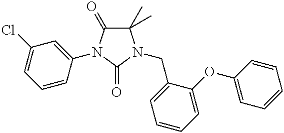 | C₂₄H₂₁ClN₂O₃ | 420.12 | 421.20 | | 2.87 | C |
| 37 | 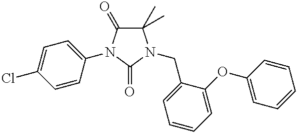 | C₂₄H₂₁ClN₂O₃ | 420.12 | 421.37 | | 2.62 | B |
| 38 | 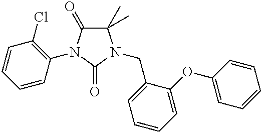 | C₂₄H₂₁ClN₂O₃ | 420.12 | | 7.68, d, 1H; 7.5, m, 4H; 7.4, m, 2H; 7.3,m, 1H; 7.15, m, 2H; 7.0, d. 2H; 6.9, d, 1H; 4.6, q. 2H; 1.4, s. 3H; 1.35, s, 3H | | |
| 39 | 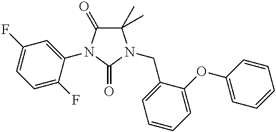 | C₂₄H₂₀F₂N₂O₃ | 422.14 | 423.14 | | 2.14 | B |
| 40 | 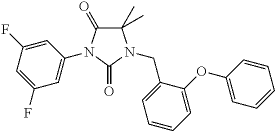 | C₂₄H₂₀F₂N₂O₃ | 422.14 | 423.14 | | 2.25 | B |
| 41 | 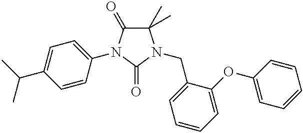 | C₂₇H₂₈N₂O₃ | 428.21 | 429.28 | | 3.07 | C |
| 42 | 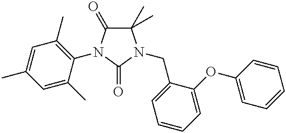 | C₂₇H₂₈N₂0₃ | 428.21 | 429.29 | | 2.91 | C |
| 43 | 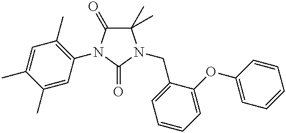 | C₂₇H₂₈N₂0₃ | 428.21 | 429.54 | | 2.68 | B |
| 44 | 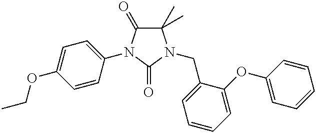 | C₂₆H₂₆N₂O₄ | 430.19 | 431.27 | | 2.77 | C |
| 45 | 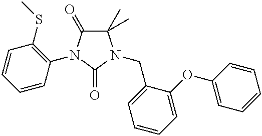 | C₂₅H₂₄N₂O₃S | 432.15 | 433.22 | | 2.63 | C |
| 46 | 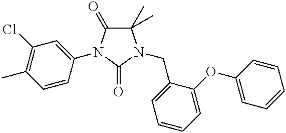 | C₂₅H₂₃ClN₂O₃ | 434.14 | 435.22 | | 2.98 | C |
| 47 | 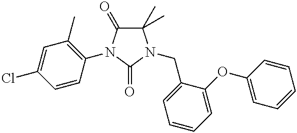 | C₂₅H₂₃ClN₂O₃ | 434.14 | 435.22 | | 2.88 | C |
| 48 | 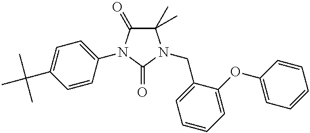 | C₂₈H₂₀N₂0₃ | 442.23 | 443.30 | | 3.18 | C |
| 49 | 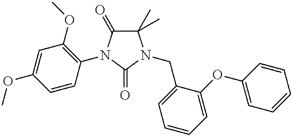 | C₂₆H₂₆N₂O₅ | 446.18 | 447.25 | | 2.57 | C |
| 50 | 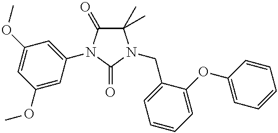 | C₂₆H₂₆N₂O₅ | 446.18 | 447.26 | | 2.66 | C |
| 51 | 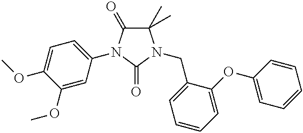 | C₂₆H₂₆N₂O₅ | 446.18 | 447.20 | | 2.48 | C |
| 52 | 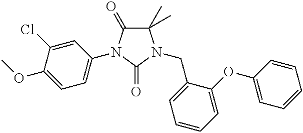 | C₂₅H₂₃ClN₂O₄ | 450.13 | 451.21 | | 2.80 | C |
| 53 | 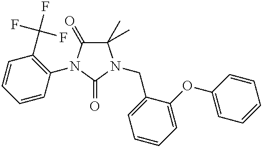 | C₂₅H₂₁F₃N₂O₃ | 454.15 | 455.23 | | 2.80 | C |
| 54 | 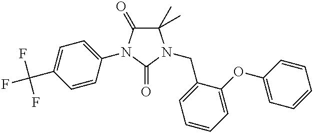 | C₂₅H₂₁F₃N₂O₃ | 454.15 | 455.51 | | 2.69 | B |
| 55 | 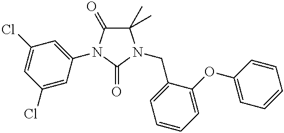 | C₂₄H₂₀Cl₂N₂O₃ | 454.09 | | 7.7, d, 1H; 7.58, m, 3H; 7.4, m, 2H; 7.3, m, 1H; 7.15, m, 2H; 7.0, d, 2H; 6.9, d, 1H; 4.6, s, 2H; 1.38, s, 6H | | |
| 56 | 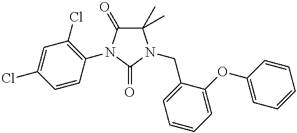 | C₂₄H₂₀Cl₂N₂O₃ | 454.09 | | 7.9, s, 1H; 7.6, m, 2H; 7.5, d, 1H; 7.4, m, 2H; 7.3, m, 1H; 7.17, m, 2H; 7.0, d, 2H; 6.9, d, 1H; 4.6, q, 2H; 1.42, s, 3H; 1.35, s, 3H? | | |
| 57 | 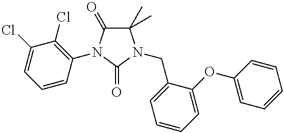 | C₂₄H₂₀Cl₂N₂O₃ | 454.09 | 455.16 | | 2.87 | C |
| 58 | 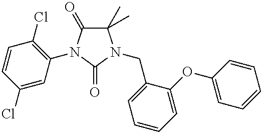 | C₂₄H₂₀Cl₂N₂O₃ | 454.09 | 455.17 | | 2.88 | C |
| 59 | 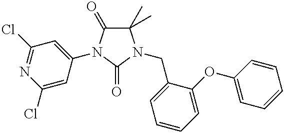 | C₂₃H₁₉Cl₂N₃O₃ | 455.08 | 456.16 | | 3.07 | C |
| 60 | 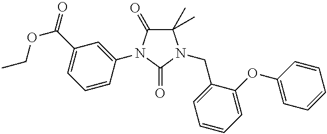 | C₂₇H₂₆N₂O₅ | 458.18 | 459.26 | | 2.85 | C |
| 61 | 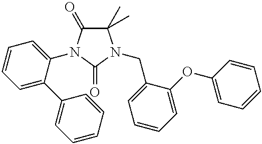 | C₃₀H₂₆N₂O₃ | 462.19 | 463.28 | | 2.90 | C |
| 62 | 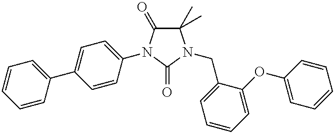 | C₃₀H₂₆N₂O₃ | 462.19 | | 7.8, d, 2H; 7.7, d, 2H; 7.6-7.48, m, 8H; 7.3, t, 1H; 7.16, m, 2H; 7.02, d, 2H; 6.9, d, 1H; 4.6. s.2H; 1.38, s, 6H | | |
| 63 | 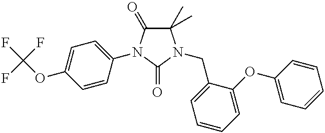 | C₂₅H₂₁F₃N₂0₄ | 470.15 | | 7.6-7.47, m, 5H; 7.4, t, 2H; 7.31, t, 1H; 7.15, m, 2H; 7.0, d, 2H; 6.9, d, 1H;4.6, s, 2H; 1.36, s, 6H | | |
| 64 | 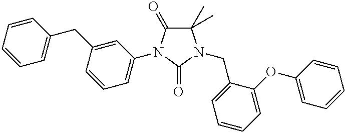 | C₃₁H₂₈N₂O₃ | 476.21 | 477.29 | | 3.07 | C |
| 65 | 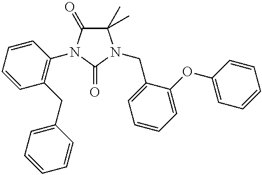 | C₃₁H₂₈N₂O₃ | 476.21 | 477.29 | | 3.08 | C |
| 66 | 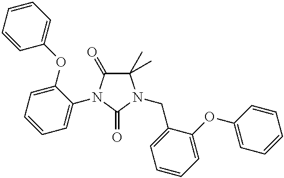 | C₃₀H₂₆N₂O₄ | 478.19 | 479.27 | | 2.93 | C |
| 67 | 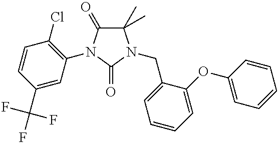 | C₂₅H₂₀ClF₃N₂O₃ | 488.11 | 489.10 | | 2.34 | B |
| 68 | 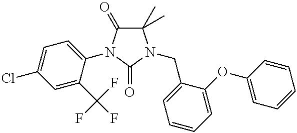 | C₂₅H₂₀ClF₃N₂O₃ | 488.11 | 489.09 | | 2.35 | B |
| 69 | 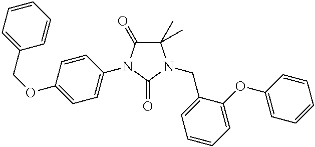 | C₃₁H₂₈N₂O₄ | 492.20 | 493.17 | | 2.38 | B |
| 70 | 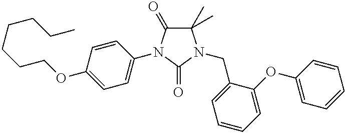 | C₃₁H₃₆N₂O₄ | 500.27 | 501.20 | | 2.77 | B |
| 71 | 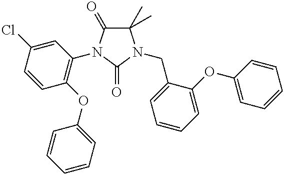 | C₃₀H₂₅ClN₂O₄ | 512.15 | 513.12 | | 2.47 | B |
| 72 | 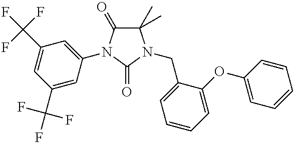 | C₂₆H₂₀F₆N₂O₃ | 522.14 | 523.12 | | 2.45 | B |
| 73 | 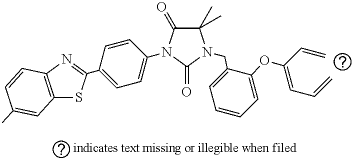 | C₃₂H₂₇N₃O₃S | 533.18 | 534.27 | | 3.32 | C |
| 74 | 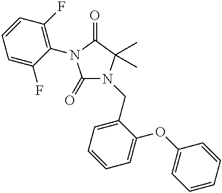 | C₂₄H₂₀F₂N₂O₃ | 422.14 | 423.26 | | 2.73 | C |
| 84 | 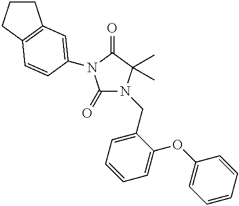 | C2₇H₂₆N₂O₃ | 426.19 | 472.27 | | 2.92 | C |

### EXAMPLE 75

### Preparation of 3-(2-chloro-6-trifluoromethylpyridin-3-yl)-5,5-dimethyl-1-(2-phenoxybenzyl)imidazolidine-2,4-dione

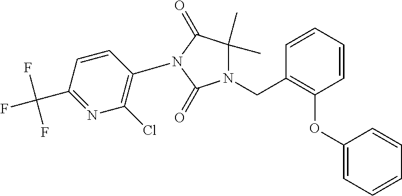

1) Preparation of 3-(2-chloro-6-trifluoromethylpyridin-3-yl)-5,5-dimethyl-imidazolidine-2,4-dione 75.1:

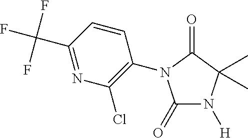

Compound 75.1 can be prepared by method “A”. To this end, 3.2 ml of phosgene solution (20% in toluene) were initially charged under argon. Then, at 75° C., 3-amino-2-chloro-6-trifluoromethylpyridine (0.62 g), dissolved in 10 ml of dry acetonitrile, was slowly added dropwise and then the mixture was stirred at 75° C. for another 90 minutes. The mixture was concentrated under reduced pressure, and the residue was admixed with toluene and concentrated again under reduced pressure. The solid residue was dissolved in 10 ml of tetrahydrofuran and 0.46 g of methyl 2-amino-2-methylpropionate hydrochloride was added. To this mixture was slowly added 0.63 ml of triethylamine. The reaction mixture was stirred at room temperature for 2 h. Thereafter, 3 ml of concentrated hydrochloric acid were added and the mixture was stirred under reflux for a further 3 h. For workup, the cooled reaction mixture was admixed slowly with saturated sodium hydrogencarbonate solution and extracted with ethyl acetate. The organic phase was dried over magnesium sulfate, filtered and concentrated under reduced pressure. After two days, the reaction product had crystallized and was washed with a little diisopropyl ether, filtered off with suction and dried under reduced pressure. This afforded 3-(2-chloro-6-trifluoromethylpyridin-3-yl)-5,5-dimethyl-imidazolidine-2,4-dione 75.1 in a yield of 47%. Molecular weight 307.03 (C₁₁H₉ClF₃N₃O₂); retention time Rₜ=1.78 min. [B]; MS (ESI): 308.35 (MH⁺). ¹H NMR: 8.85, s, 1H, 8.41, d, 1H, 8.2, d, 1H, 1.48, 2s, 6H.

2) Alkylation of 75.1 with 1.2 to obtain 75:

62 mg of compound 75.1 were dissolved at room temperature in 2 ml of dry dimethylformamide and admixed successively with 58 mg of 1-bromomethyl-2-phenoxybenzene (1.2) and 72 mg of cesium carbonate and stirred at 80° C. for 4 h. For workup, the cooled reaction mixture was filtered through a syringe filter and purified by chromatography (method [RP1]). This afforded 3-(2-chloro-6-trifluoromethyl-pyridin-3-yl)-5,5-dimethyl-1-(2-phenoxybenzyl)-imidazolidine-2,4-dione 75 in a yield of 82%. Molecular weight 489.10 (C₂₄H₁₉ClF₃N₃O₃); retention time Rₜ=2.69 min. [B]; MS (ESI): 490.41 (MH⁺).

The imidazolidin-2,4-diones 76.1-83.1 and 85.1-87.1, which serve as starting materials for compounds of examples 76-83 and 85-87, were obtained by reaction of tert-butyl 2-amino-2-methylpropionate hydrochloride or the corresponding methyl ester with the particular isocyanates as described above for compound 75.1.

For instance, 76.1 (5,5-dimethyl-3-(2-phenoxyphenyl)imidazolidine-2,4-dione; ¹H NMR: 8.4, s, 1H, 7.43, m, 2H, 7.3, m, 3H, 7.1, m, 2H, 6.9, d, 2H, 1.38, s, 3H, 1.09, s, 3H) was obtained by reaction with 1-isocyanato-2-phenoxybenzene,

77.1 (3-(2-chloro-4-methanesulfonylphenyl)-5,5-dimethylimidazolidine-2,4-dione; molecular weight 316.02 (C₁₂H₁₃ClN₂O₄S); retention time Rₜ=1.22 min. [α]; MS (ESI): 317.06 (MH⁺)) by reaction with 2-chloro-1-isocyanato-4-methanesulfonylbenzene;

78.1 (5,5-dimethyl-3-(3-phenoxyphenyl)imidazolidine-2,4-dione; molecular weight 296.11 (C₁₇H₁₆N₂O₃); retention time Rₜ=1.94 min. [B]; MS (ESI): 297.48 (MH⁺)) by reaction with 1-iso cyanato-3-phenoxybenzene;

80.1 (5,5-dimethyl-3-(4-phenoxyphenyl)imidazolidine-2,4-dione; molecular weight 296.11 (C₁₇H₁₆N₂O₃); retention time Rₜ=1.96 min. [B]; MS (ESI): 297.48 (MH⁺)) by reaction with 1-isocyanato-4-phenoxybenzene;

82.1 (3-(2-chloropyridin-4-yl)-5,5-dimethylimidazolidine-2,4-dione; molecular weight 239.04 (C₁₀H₁₀ClN₃O₂); retention time Rₜ=1.07 min. [B]; MS (ESI): 240.06 (MH⁺)) by reaction with 2-chloro-4-isocyanatopyridine;

83.1 (5,5-dimethyl-3-(2-trifluoromethylpyridin-4-yl)imidazolidine-2,4-dione; molecular weight 273.07 (C₁₁H₁₀F₃N₃O₂); retention time Rₜ=1.62 min. [B]; MS (ESI): 274.33 (MH⁺)) by reaction with 4-isocyanato-2-trifluoromethylpyridine;

85.1 (3-(2-ethoxypyridin-4-yl)-5,5-dimethylimidazolidine-2,4-dione; molecular weight 249.11 (C₁₂H₁₅N₃O₃); retention time R_{t=1.06} min. [B]; MS (ESI): 250.08 (MH⁺)) by reaction with 2-ethoxy-4-isocyanatopyridine;

86.1 (5-(4,4-dimethyl-2,5-dioxoimidazolidin-1-yl)-pyridine-2-carbonitrile; molecular weight 230.08 (C₁₁H₁₀N₄O₂); retention time R_{t=1.31} min. [B]; MS (ESI): 231.24 (MH⁺)) by reaction with 5-isocyanatopyridine-2-carbonitrile;

87.1 (5,5-dimethyl-3-(4-methylsulfanyl-3-trifluoromethylphenyl)imidazolidine-2,4-dione; molecular weight 318.06 (C₁₃H₁₃F₃N₂O₂S); retention time R_{t=1.93} min. [B]; MS (ESI⁻): 317.07 (M-H′)) by reaction with 4-isocyanato-1-methylsulfanyl-2-trifluoromethylbenzene;

92.1 (4-(4,4-dimethyl-2,5-dioxoimidazolidin-1-yl)-2-trifluoromethylbenzonitrile; melting point 208-211° C.;

by reaction with 4-isocyanato-2-trifluoromethylbenzonitrile.

The following compounds (table 2) of examples 76-83 and 85-87 and 96 were obtained by alkylation of compounds 76.1.-83.1 and 85.1-87.1 and 92.1 with the following benzyl bromides:

example 76: reaction of 76.1 with 3-(4-fluorophenoxy)benzyl bromide (76.2);

example 77: reaction of 77.1 with compound 1.2;

example 78: reaction of 78.1 with 3-(4-fluorophenoxy)benzyl bromide (76.2);

example 79: reaction of 78.1 with 3-(2-fluorophenoxy)benzyl bromide (79.2);

example 80: reaction of 80.1 with 3-(4-fluorophenoxy)benzyl bromide (76.2);

example 81: reaction of 80.1 with 3-(2-fluorophenoxy)benzyl bromide (79.2);

example 82: reaction of 82.1 with compound 1.2;

example 83: reaction of 83.1 with compound 1.2;

example 85: reaction of 85.1 with compound 1.2;

example 86: reaction of 86.1 with compound 1.2;

example 87: reaction of 87.1 with compound 1.2;

example 96: reaction of 92.1 with methyl 4-(2-bromomethylphenoxy)benzoate (96.1; ¹H NMR: 8.0, d, 2H, 7.61, d, 1H, 7.4, m, 1H, 7.27, t, 1H, 7.08, m, 3H, 4.67, s, 2H, 3.85, s, 3H; prepared from methyl 4-o-tolyloxybenzoate (96.2; ¹H NMR: 7.95, d, 2H, 7.39, d, 1H, 7.3, m, 1H, 7.2, t, 1H, 7.05, d, 1H, 6.92, d, 2H, 3.81, s, 3H, 2.1, s, 3H) by bromination with N-bromosuccinimide)).

Table 2

| | | | | | | Reten- | |
|---|---|---|---|---|---|---|---|
| Exam- | | | | | | tion | HPLC/ |
| ple | | Empirical | Molecular | | | time | MS |
| No. | Product | formula | weight | MH⁺ | ¹H NMR | T_{R} [min.] | method |
| 76 | 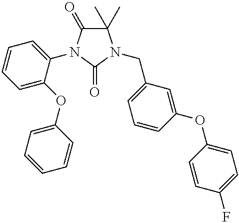 | C₃₀H₂₅FN₂O₄ | 496.17 | 497.17 | | 2.27 | B |
| 77 | 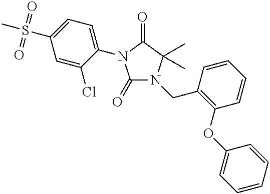 | C₂₅H₂₃ClN₂O₅S | 498.10 | | 8.21, s, 1H; 8.08, d, 1H; 7.9, d. 1H; 7.55, d, 1H; 7.4, m, 2H; 7.33, m, 1H; 7.18, m, 2H; 7.02, d, 2H; 6.9, d, 1H; 4.62, q, 2H; 1.45, s,3H; 1.38, s, 3H | | |
| 78 | 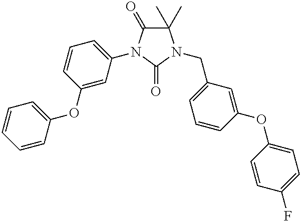 | C₃₀H₂₅FN₂O₄ | 496.17 | 497.18 | | 2.36 | B |
| 79 | 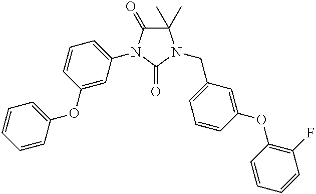 | C₃₀H₂₅FN₂O₄ | 496.17 | 497.17 | | 2.33 | B |
| 80 | 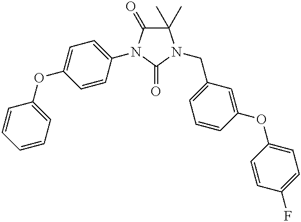 | C₃₀H₂₅FN₂O₄ | 496.17 | 497.15 | | 2.32 | B |
| 81 | 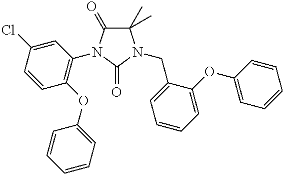 | C₃₀H₂₅FN₂O₄ | 496.17 | 497.47 | | 2.69 | B |
| 82 | 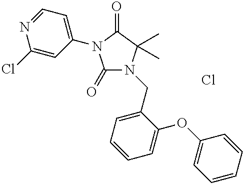 | C₂₃H₂₀ClN₃O₃ × HCl | 421.11 (free base) | 422.11 | | 2.13 | B |
| 83 | 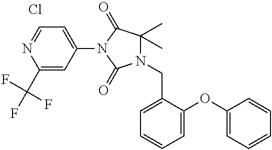 | C₂₄H₂₀F₃N₃O₃ × HCl | 455.14 (free base) | 456.06 | | 2.21 | B |
| 85 | 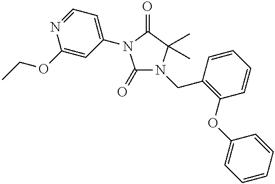 | C₂₅H₂₅N₃O₄ | 431.18 | 432.19 | | 2.15 | B |
| 86 | 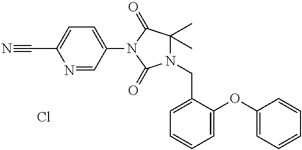 | C₂₄H₂₀N₄O₃ | 412.53 | 413.27 | | 2.55 | C |
| 87 | 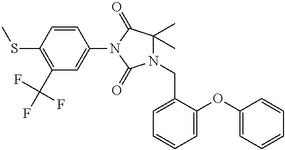 | C₂₆H₂₃F₃N₂O₃S | 500.13 | 501.14 | | 2.36 | B |
| 96 | 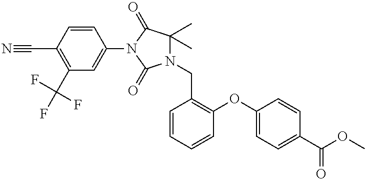 | C₂₈H₂₂F₃N₃O₅ | 537.15 | | 8.31, d. 1H; 8.17, s, 1H; 8.04, d, 1H; 7.9, d, 2H; 7.63, d, 1H; 7.4, m, 1H; 7.28, t, 1H; 7.08, m, 3H; 4.58, s, 2H; 3.83, s, 3H; 1.4, s, 6H | | |

Compound 88.1 (3-(4-methanesulfonyl-3-trifluoromethylphenyl)-5,5-dimethylimidazolidine-2,4-dione; molecular weight 350.05 (C₁₃H₁₃F₃N₂O₄S); retention time R_{t=1.83} min. [B]; MS (ESI): 392.22 (MH⁺+CH₃CN)) was obtained by oxidation of 87.1 with m-chloroperbenzoic acid.

### EXAMPLE 88

### Reaction of 88.1 with compound 1.2

| Exam- | | | | | | Retention | HPLC/ |
|---|---|---|---|---|---|---|---|
| ple | | Empirical | Molecular | | | time | MS |
| No. | Product | formula | weight | MH⁺ | ¹H NMR | T_{R} [min.] | method |
| 88 | 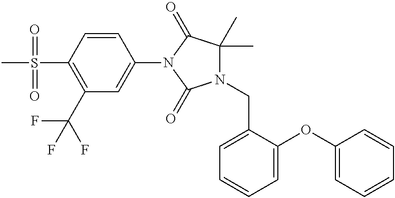 | C₂₆H₂₃F₃N₂O₅S | 532.12 | 533.15 | | 2.13 | B |

### EXAMPLE 84

### 4-[2,5-Dioxo-3-(2-phenoxybenzyl)-4-phenylimidazolidin-1-yl]-2-trifluoromethylbenzonitrile

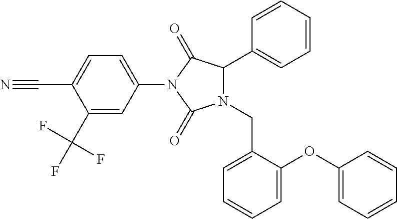

1) Preparation of 4-(2,5-dioxo-4-phenylimidazolidin-1-yl)-2-trifluoromethyl-benzonitrile 84.1:

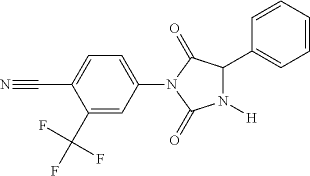

5.3 ml of a solution of phosgene in toluene (20% phosgene in toluene) were initially charged under argon and admixed dropwise at 75° C. with a solution of 1.0 g of 4-amino-2-trifluoromethylbenzonitrile in 25 ml of dry acetonitrile. After the addition had ended, the reaction mixture was stirred at 75° C. for 2 h. The cooled reaction mixture was concentrated under reduced pressure, and the residue was admixed with toluene and concentrated again under reduced pressure. Thereafter, the residue was dissolved in 20 ml of tetrahydrofuran and admixed with 1.0 g of methyl 2-amino-phenylacetate. To this mixture were slowly added dropwise 1.05 ml of triethylamine and then the mixture was stirred at room temperature for 12 h. Finally, the reaction mixture was admixed with 5 ml of concentrated hydrochloric acid and stirred under reflux for 8 h. For workup, the mixture was admixed with saturated sodium hydrogencarbonate solution and extracted with ethyl acetate. The organic phase was dried over magnesium sulfate, filtered and concentrated under reduced pressure. The residue was purified by chromatography (method [RP1]). This afforded 4-(2,5-dioxo-4-phenylimidazolidin-1-yl)-2-trifluoromethylbenzonitrile; molecular weight 345.07 (C₁₇H₁₀F₃N₃O₂); retention time R_{t=2.05} min. [B]; MS (ESI⁻): 344.51 (M-H⁺).

2) Preparation of 4-[2,5-dioxo-3-(2-phenoxybenzyl)-4-phenylimidazolidin-1-yl]-2-trifluoromethylbenzonitrile:

The alkylating reaction of compound 84.1 with 1.2 under conditions as described above afforded compound 84. ¹H NMR: 9.5, s, 1H, 8.25, d, 1H, 7.75-6.7, m, 16H, 3.75, d, 1H, 3.37, d, 1H.

### EXAMPLE 95

### 4-[3-(2-Chloro-6-phenoxybenzyl)-2,5-dioxo-4-phenylimidazolidin-1-yl]-2-trifluoromethylbenzonitrile

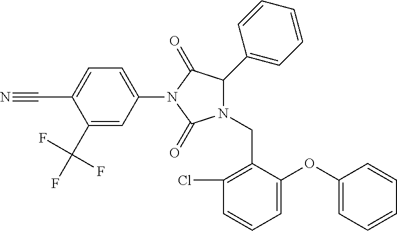

The compound of example 95 was obtained by reaction of 84.1 with 2-bromomethyl-1-chloro-3-phenoxybenzene analogously to the procedure for the preparation of 84.

### EXAMPLE 97

### 4-{2-[3-(4-Cyano-3-trifluoromethylphenyl)-5,5-dimethyl-2,4-dioxo-imidazolidin-1-ylmethyl]phenoxy}benzoic acid

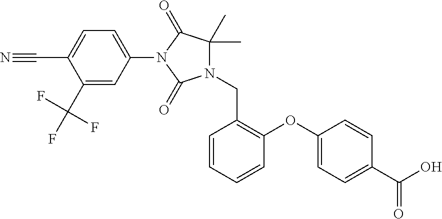

The acid of example 97 was obtained by acidic hydrolysis (conc. hydrochloric acid, dioxane, 80° C., 8 h) from the ester 96. Molecular weight 523.13 (C₂₇H₂₀F₃N₃O₅); retention time R_{t=2.32} min. [B]; MS (ESI⁻): 522.13 (M-H⁺).

### EXAMPLE 98

### 4-{2-[3-(4-Cyano-3-trifluoromethylphenyl)-5,5-dimethyl-2,4-dioxo-imidazolidin-1-ylmethyl]phenoxy}benzamide

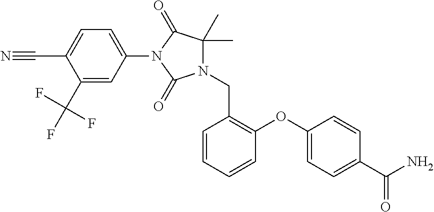

The reaction of the ester 96 with a 7 molar solution of ammonia in methanol afforded, after 12 weeks at room temperature, the primary amide 98. Molecular weight 522.15 (C₂₇H₂₁F₃N₄O₄); retention time R_{t=1.84} min. [B]; MS (ESI): 523.29 (MH⁺).

### EXAMPLE 105

### 4-{3-[2-(4-Chlorophenylsulfanyl)benzyl]-4,4-dimethyl-2,5-dioxoimidazolidin-1-yl}-2-trifluoromethylbenzonitrile

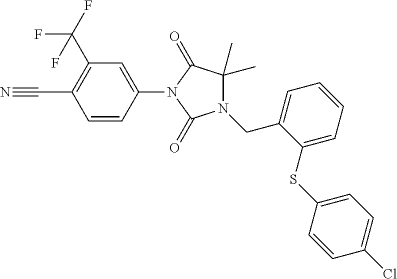

1) Preparation of 2-(4-chlorophenylsulfanyl)benzyl bromide 105.1:

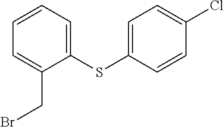

2.85 g of triphenylphosphine and 0.88 g of imidazole were dissolved in 25 ml of dichloromethane; at 5° C., a solution of 1.73 g of bromine in 5 ml of dichloromethane was added dropwise and, on completion of addition, the mixture was stirred at 5° C. for 10 min. To this solution was added dropwise a solution of 2.59 g of [2-(4-chlorophenylsulfanyl)phenyl]methanol in 20 ml of dichloromethane. The reaction mixture was stirred at room temperature for 2 days. For workup, the reaction mixture was admixed with 25 ml of 1 N hydrochloric acid. The organic phase was removed, dried over magnesium sulfate, filtered and purified by chromatography (silica gel; 3/1 n-heptane/ethyl acetate). This afforded 2-(4-chlorophenylsulfanyl)benzyl bromide 105.1. ¹H NMR: 7.61, d, 1H, 7.44, d, 2H, 7.35, m, 2H, 7.28, m, 3H, 4.81, s, 2H.

2) 4-{3-[2-(4-Chlorophenylsulfanyl)benzyl]-4,4-dimethyl-2,5-dioxoimidazolidin-1-yl}-2-trifluoromethylbenzonitrile 105:

300 mg of compound 92.1 and 317 mg of 105.1 were dissolved in 3 ml of dry acetonitrile, admixed with 330 mg of cesium carbonate and stirred at room temperature

for 4 h. For workup, the reaction mixture was admixed with a little ethyl acetate and water; the organic phase was removed, dried over magnesium sulfate, filtered and concentrated under reduced pressure. Chromatographic purification (method [RP1]) afforded 4-{3-[2-(4-chloro-phenylsulfanyl)benzyl]-4,4-dimethyl-2,5-dioxo-imidazolidin-1-yl}-2-trifluoromethylbenzonitrile 105. ¹H NMR: 8.34, d, 1H, 8.22, s, 1H, 8.09, d, 1H, 7.61, d, 1H, 7.46-7.35, m, 5H, 7.2, d, 2H, 4.65, s, 2H, 1.35, s, 6H.

### EXAMPLE 106

### 4-{3-[2-(4-Chlorobenzenesulfonyl)benzyl]-4,4-dimethyl-2,5-dioxoimidazolidin-1-yl}-2-trifluoromethylbenzonitrile

### and

### EXAMPLE 107

### 4-{3-[2-(4-Chlorobenzenesulfinyl)benzyl]-4,4-dimethyl-2,5-dioxoimidazolidin-1-yl}-2-trifluoromethylbenzonitrile

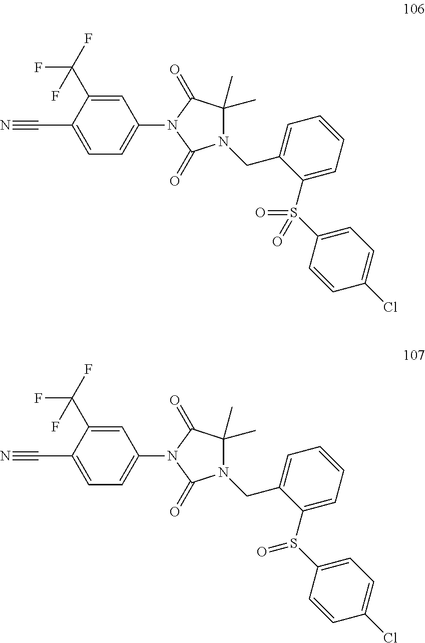

150 mg of the compound of example 105 were dissolved in a mixture of 10 ml of methanol and 2 ml of water, admixed with 2 equivalents of potassium peroxodisulfate and stirred at room temperature for 4 h. For workup, the methanol was removed under reduced pressure, the residue was admixed with water and dichloromethane, and the organic phase was removed and dried over magnesium sulfate. After filtration, the organic phase was removed and concentrated under reduced pressure, and the residue was purified by chromatography (method [RP1]). This afforded 30% 4-{3-[2-(4-chlorobenzenesulfonyl)benzyl]-4,4-dimethyl-2,5-dioxo-imidazolidin-1-yl}-2-trifluoromethylbenzonitrile 106 (¹H NMR: 8.34, d, 1H, 8.21, s, 1H, 8.15, d, 1H, 8.09, d, 1H, 7.95, d, 2H, 7.75, m, 4H, 7.63, m, 1H, 4.72, s, 2H, 1.26, s, 6H) and 40% 4-{3-[2-(4-chlorobenzenesulfinyl)benzyl]-4,4-dimethyl-2,5-dioxoimidazolidin-1-yl}-2-trifluoromethylbenzonitrile 107 (molecular weight 545.07 (C₂₆H₁₉ClF₃N₃O₃S); retention time R_{t=2.09} min. [B]; MS (ESI): 546.27 (MH⁺).

Pharmacological Testing:

In Vitro Tests:

In vitro functional assays with recombinant cells:

Function-testing assays were performed by means of the FLIPR technique (“Fluorometric Imaging Plate Reader”, Molecular Devices Corp.).

To this end, ligand-induced changes in the intracellular concentration of Ca²⁺ in recombinant HEK293 cells, which expressed both a cannabinoid receptor (CB1 or CB2) and G-protein Galpha16, were determined. For the studies, cells were sown into 96-well microtiter plates (60 000 cells/well) and left to grow overnight. The medium was removed and the cells were incubated in buffer which contained the fluorescent dye Fluo-4. After this loading with dye, the cells were washed, test substance was added dissolved in buffer, the mixture was incubated for 20 minutes, a known cannabinoid receptor agonist as a reference agonist was added in buffer and, finally, the changes in the intracellular Ca²⁺ concentration were measured in the FLIPR unit.

Results were presented as the percentage change relative to the control (0%: analogous experiment without test substance and without reference agonist, i.e. only with buffer; 100%: analogous experiment without test substance, but with reference agonist in excess), and used to calculate dose/action curves, and IC₅₀ values were determined.

Results:

The values of the functional assay compared to the cannabinoid 1 receptor including illustrative selectivities compared to the cannabinoid 2 receptor can be taken from table 3 which follows.

TABLE 3

| Example No. | hCB1R: FLIPR; IC₅₀ [nM] | hCB2R: FLIPR; IC₅₀ [nM] |
|---|---|---|
| 82 | 100 | >10000 |
| 96 | 152 | |
| 97 | | >10000 |
| 98 | 507 | >10000 |
| 105 | | >10000 |

Binding to the CB1 Receptor:

Test compounds: The compounds (3 μl, 10 mM, 100% DMSO), pipetted into 96-well PP microtiter plates, were diluted with 27 μl of 100% DMSO (dimethyl sulfoxide). Proceeding from this solution, further 3-fold dilution steps were undertaken by transferring 10 μl in each case to a new PP microtiter plate and adding a further 20 μl of 100% DMSO. In each case 6 μl of these solutions were transferred into new 96-well PP microtiter plates and made up with 144 μl of assay buffer. The end concentrations ranged from 10 μM to 0.005 μM.

Negative control: AM 251, dissolved in assay buffer with 1% DMSO, was added to the dilution series in the microtiter plates as a control. The end concentration was 1 μM.

Blank control: assay buffer with 1% DMSO was added to the dilution series of the microtiter plates as a blank control.

Summary of the Assay Parameters:

| | | |
|---|---|---|
| Assay volume | | 200 μl |
| Receptor | CHO-K1/cannabinoid CB1 | 2 μg/well |
| | Protein | |
| Ligand | [³H]-SR141716A | 0.5 nM |
| | | 0.0195 μCi/well |
| Ions | Tris-HCl | 50 mM, pH 7.4 |
| | MgCl₂ | 5 mM |
| | EDTA | 2.5 mM |
| | BSA (fatty acid-free) | 0.2% |
| Nonspecific binding | AM 251 | 1 μM |
| Compound | in 1% DMSO | 10 μM to 0.0050 μM |

Analysis of the data:
High control: ³H binding without addition of the compound
Low control: ³H binding in the presence of 1 μM AM 251

The values were calculated using the corrected raw data.

$$Inhibition of ligand binding ( \% ) = 100 * ( 1 - \frac{( sample - lowcontrol )}{( highcontrol - lowcontrol )} )$$

The values reported were obtained as average values of a double determination. The IC₅₀ values were calculated from the measurements with the program Xlfit, formula 205. Ki values were obtained from the IC₅₀ and Kd values utilizing the Cheng-Prusoff equation:

$$Ki = \frac{IC 50}{1 + \frac{C}{Kd}} ( C = concentration of the radioligand )$$

Literature: Cheng, Y.-C., and Prusoff, W. H. (1973) Biochem. Pharmacol 22, 3099-3108 Results: Kᵢ values of example compounds;

TABLE 4

| Example No. | hCB1R; binding Kᵢ [nM] |
|---|---|
| 8 | 249 |
| 12 | 284 |
| 14 | 289 |
| 82 | 2 |
| 83 | 24 |
| 86 | 311 |
| 96 | 22 |
| 97 | 533 |
| 105 | 44 |
| 107 | 232 |

It can be seen from the test data that the inventive compounds of the formula I act as CB1R antagonists and are therefore very suitable for treating metabolic syndrome, type II diabetes and obesity.

In Vivo Tests:

“Milk consumption in mice”

The test is used to study the anorexigenic potency of the test substances. Female NMRI mice, 25-35 g in weight, are used. The mice are accustomed to the housing conditions for at least one week and to the condensed milk supplied for 2 days.

The feed is removed from the mice for 24 hours, but they have constant access to water. On the day of the experiment, the animals are put in individual cages; the cage lids can accommodate the pipettes filled with milk. The test substances are administered orally, intraperitoneally or subcutaneously. After the administration, the mice are put in their cages and receive access to the milk 30 min later. The milk consumption is read off every 30 min over 7 hours; at the same time, obvious changes in behavior of the animals are noted.

“Antagonization of CB1-Mediated Hypothermia”

The test is used to measure the potency of cannabinoid CB1 receptor (CB1) antagonists. What is measured is the extent to which the CB1 antagonists to be tested are capable of preventing or of antagonizing hypothermia induced by a CB1 agonist.

Female NMRI mice, 25-35 g in weight, are used. The mice are accustomed to the housing conditions for at least one week.

At time 0 min, the animals are treated orally, intravenously or intraperitoneally with the CB1 antagonist to be tested. 30 min later, the CB1 agonist CP55.940, 1.25 mg/kg, is administered to the mice intraperitoneally. This brings about a fall in the body temperature by 5-6° C. within 30 min. The body temperature is measured rectally for the first time 30 min before the test substance administration and then every 30 min after this administration, if appropriate immediately before a substance administration, over 4 hours.

The potency of the test substances is reported as the percentage decrease in the area under the temperature-time curve which is formed firstly by the average basal temperature, and secondly by the temperature-time curve, of the animals treated exclusively with the CB1 antagonist.

“Intestinal Motility in Mice”

The method serves firstly to study the influence of test substances themselves on the small intestinal motility, and secondly to study to what extent specifically induced effects on the small intestinal motility can be prevented or antagonized, for example the delay in the intestinal passage by the cannabinoid CB1 agonist CP55.940.

Female NMRI mice with a weight of 25-35 g are used. The mice are accustomed to the housing conditions for at least one week.

The feed is removed from the mice for 24 hours, but they have constant access to water. The test substances are administered orally, intravenously, subcutaneously, but not intraperitoneally. If a specific effect is to be antagonized, the test substance is administered 30-120 min before the specific effector. 30 min after this administration, a defined amount of a dyed, non-caloric filler is introduced into the stomach by gavage. After a further 30 min (the dyed filler has about 80% filled the small intestine at this point), the animals are sacrificed and the small intestine is dissected. The intestinal motility is reported as the passage of the dyed filler compared to the total length of the small intestine in percent. A treatment effect is reported as the difference of this passage to the vehicle control, likewise in percent.

1. A compound of the formula I 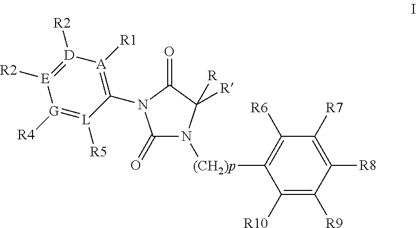
in which
R, R′ are each independently H, (CH₂)ₙ-aryl, (C₁-C₆)-alkyl, where (C₁-C₆)-alkyl or the aryl radical may be substituted by halogen, O-R14, S(O)ₘ-R12 or NR13R15;
or R and R′ together form a ring having from three to eight carbon atoms, where one carbon atom may be replaced by O, S(O)ₘ, N—(CH₂)ₙ—CO—NH-aryl, NR13 or NR15;
m is 0, 1, 2;
n is 0, 1, 2, 3, 4;
p is 1, 2, 3, 4, 5;
q is 1, 2, 3, 4;
r is 2, 3, 4, 5, 6;
v is 0, 1, 2, 3, 4;
A, D, E, G, L are each independently C or N, where there is no corresponding R1, R2, R3, R4, R5 substituent when they are defined as N, or R2-D=E-R3 or R4-G=L-R5 are defined as S or O;
R1, R2, R3, R4, R5 are each independently H, F, Cl, Br, I, CN, N₃, NC, NO₂, CF₃, (C₁-C₈)-alkyl, (C₃-C₈)-cycloalkyl, (CH₂)_{q}-[(C₃-C₈)-cycloalkyl], (CH₂)ₙ-[(C₃-C₈)-cycloalkenyl], (CH₂)ₙ-[(C₇-C₁₂)-bicycloalkyl], (CH₂)ₙ-[(C₇-C₁₂)-bicycloalkenyl], (CH₂)ₙ-[(C₇-C₁₂)-tricycloalkyl], adamantan-1-yl, adamantan-2-yl, (CH₂)ₙ-aryl, (CH₂)ₙ-heteroaryl, OCF₃, O-R11, NR13R15, NH—CN, S(O)ₘ-R12, SO₂—NH₂, O₂—N═CH—N(CH₃)₂, SO₂—NH—CO-R12, SO₂—NH—CO—NHR12, SO₂—NH—CO-R16, SO₂—NH—[(C₁-C₈)-alkyl], SO₂—NH—[(C₃-C₈)-cycloalkyl], SO₂—NH—(CH₂)ᵣ—OH, SO₂—NH—(CH₂)ₙ-aryl, SO₂—NH—(CH₂)ₙ-heteroaryl, SO₂—N[(C₁-C₈)-alkyl]₂, SO₂—N[(CH₂)ₙ-aryl][(CH₂)ₙ-heteroaryl], SO₂-R16, SF₅, CO—O[(C₁-C₈)-alkyl], CO—O[(C₃-C₈)-cycloalkyl], CO—O—(CH₂)ᵣ—NH₂, CO—O—(CH₂)ₙ-aryl, CO—O—(CH₂)ₙ-heteroaryl, CO—NH₂, CO—NH—CN, CO—NH—[(C₁-C₈)-alkyl], CO—NH—(CH₂)ᵣ—OH, CO—N[(C₁-C₈)-alkyl]₂, CO—NH—[(C₃-C₈)-cycloalkyl], CO—N[(C₃-C₈)-cycloalkyl]₂, C(═NH)—O—[(C₁-C₆-alkyl)], C(═NH)—NH₂, C(═NH)—NR12R13, C(═NH)—R16, C(═NR13)-NR12R13, (CH₂)ₙ—C(═NSO₂-R12)NH₂, CO—NH—SO₂-R16, CO—NH—SO₂—NHR12, CO-R16, COOH, CO—(C₁-C₈)-alkyl, CO—(C₃-C₈)-cycloalkyl, CO—(CH₂)ₙ-[(C₇-C₁₂)-bicycloalkyl], CO—(CH₂)ₙ-[(C₇-C₁₂)-tricycloalkyl], CO-aryl, CO-heteroaryl, CH(OH)-aryl, CH(OH)-heteroaryl, CH[O—(C₁-C₆)-alkyl]-aryl, CH[O—(C₁-C₆)-alkyl]-heteroaryl, CHF-aryl, CHF-heteroaryl, CF₂-aryl, CF₂-heteroaryl, CHO, CH₂—OH, CH₂—CN, CH₂—O-R12, CH₂—O—(CH₂)ₙ—CO—O[(C₁-C₈)-alkyl], CH₂—O—(CH₂)ₙ—CO—NH₂, CH₂—O—(CH₂)_{q}—COOH,
where the alkyl, cycloalkyl, cycloalkenyl, bicycloalkyl, bicycloalkenyl and tricycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, (CH₂)ₙ-aryl, O—(CH₂)ₙ-aryl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms; and where the R1 and R2, R2 and R3, R3 and R4 or R4 and F5 radicals in each case together be defined as —(CH₂)₃— or —(CH₂)₄— or —CH═CH—CH═CH—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or the X-bicyclic heterocycle may be unsubstituted or mono- or polysubstituted by
R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ₙ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ-R12, SO₂-R16, SO₂—N═CH—N(CH₃)₂, 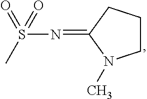
SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—-[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}—[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂, 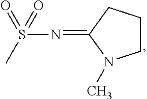
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as X-bicyclic heterocycle, X-aryl or X-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ
R11 is H, (C₁-C₈)-alkyl, (C₂-C₁₀)-alkenyl, (C₂-C₁₀)-alkynyl, (C₃-C₈)-cycloalkyl, (CH₂)_{q}—[(C₃-C₈)-cycloalkyl], (CH₂)ₙ-[(C₇-C₁₂)-bicycloalkyl], (CH₂)ₙ-[(C₃-C₁₀)-cycloalkenyl], (CH₂)ₙ-[(C₃-C₁₀)-bicycloalkenyl], (CH₂)ₙ—[(C₇-C₁₂)-tricycloalkyl], (CH₂)ₙ-aryl, (CH₂)ₙ—CO—[O—(C₁-C₈)-alkyl], (CH₂)ₙ—CO—[O—(C₃-C₈)-cycloalkyl], (CH₂)₂—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)_{q}—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (CH₂)ₙ—CO—N[(C₃-C₈)-cycloalkyl]₂, (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CR21[(CO—O(C₁-C₆)-alkyl)]₂, (CH₂)ₙ—CR21(CONH₂)₂, (CH₂)ₙ—CR21(COOH)₂, (CH₂)ₙ—CR21R22CO—O[(C₁-C₆)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₈)-alkyl], (CH₂)ₙ—C(CH₃)₂—CO—O[(C₃-C₈)-cycloalkyl], (CH₂)ₙ—C(CH₃)₂—CO—O—(CH₂)ᵣ—NH₂, (CH₂)ₙ—C(CH₃)₂—CO—O—(CH₂)ₙ-aryl, (CH₂)ₙ—C(CH₃)₂—CO—O—(CH₂)ₙ-heteroaryl, (CH₂)ₙ—C(CH₃)₂—CO—NH₂, (CH₂)ₙ—C(CH₃)₂—CO—NH—[(C₁-C₈)-alkyl], (CH₂)ₙ—C(CH₃)₂—CO—NH—(CH₂)ᵣ—OH, (CH₂)ₙ—C(CH₃)₂—CO—N[(C₁-C₈)-alkyl]₂, (CH₂)ₙ—C(CH₃)₂—CO—NH—[(C₃-C₈)-cycloalkyl], (CH₂)ₙ—C(CH₃)₂—CO—N[(C₃-C₈)-cycloalkyl]₂, (CH₂)ₙ—C(CH₃)₂—COOH, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl, bicycloalkyl, cycloalkenyl and bicycloalkenyl radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R12 is H, (C₁-C₈)-alkyl, (C₃-C₈)-cycloalkyl, (CH₂)_{q}—[(C₃-C₈)-cycloalkyl], (CH₂)ₙ—[(C₇-C₁₂)-bicycloalkyl], (CH₂)ₙ-[(C₇-C₁₂)-tricycloalkyl], (CH₂)ₙ-aryl, (CH₂)ₙ-heteroaryl, where the alkyl or cycloalkyl radicals may be substituted by fluorine atoms,
and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R13 is H, SO₂—[(C₁-C₈)-alkyl], SO₂—[(C₃-C₈)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂, where the alkyl and cycloalkyl radicals may be substituted by fluorine atoms and where the aryl or heteroaryl radical may be substituted by halogen, CN, CF₃, (C₁-C₆)-alkyl, (C₃-C₆)-cycloalkyl, O—[(C₁-C₆)-alkyl], S(O)ₘ-[(C₁-C₆)-alkyl], SO₂—NH₂, COOH, CONH₂, CO—[O(C₁-C₆)-alkyl], CO—(C₁-C₆)-alkyl and where the alkyl radicals may be substituted by fluorine atoms;
R14 is H, (C₁-C₈)-alkyl, (C₃-C₈)-cycloalkyl, (CH₂)_{q}-[(C₃-C₈)-cycloalkyl], (CH₂)ₙ-aryl, (CH₂)ₙ-heteroaryl, (CH₂)ₙ—CO—[O—(C₁-C₈)-alkyl], (CH₂)ₙ—CO—[O—(C₃-C₈)-cycloalkyl], (CH₂)ₙ—CO—[O—(CH₂)ₙ-aryl], (CH₂)ₙ—CO—[O—(CH₂)ₙ-heteroaryl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—SO₂—NH₂, (CH₂)ₙ—CO—NH—[(C₁-C₈)-alkyl], (CH₂)ₙ—CO—N[(C₁-C₈)-alkyl], (CH₂)ₙ—CO—NH—[(C₃-C₈)-cycloalkyl], (CH₂)ₙ—CO—N[(C₃-C₈)-cycloalkyl]₂, (CH₂)ₙ—C(CH₃)₂—CO—O[(C₁-C₈)]-alkyl, (CH₂)ₙ—C(CH₃)₂—CO—O[(C₃-C₈)]-cycloalkyl, (CH₂)ₙ—C(CH₃)₂—CO—O—(CH₂)ᵣ—NH₂, (CH₂)ₙ—C(CH₃)₂—CO—NH₂, (CH₂)ₙ—C(CH₃)₂—CO—NH—(CH₂)ᵣ—OH, (CH₂)ₙ—C(CH₃)₂—COOH, where the alkyl and cycloalkyl radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R15 is H, (C₁-C₈)-alkyl, (C₃-C₈)-cycloalkyl, (CH₂)ₙ-aryl, (CH₂)ₙ-heteroaryl, (CH₂)ₙ—CO—[O—(C₁-C₈)-alkyl], (CH₂)ₙ—CO—[O—(C3-C8)-cycloalkyl], (CH₂)ₙ—CO—[O—(CH₂)ₙ-aryl], (CH₂)ₙ—CO—[O—(CH₂)ₙ-heteroaryl], CO—[(C₁-C₈)-alkyl], CO—[(C3-C8)-cycloalkyl], CO-aryl, CO-heteroaryl, (CH₂)ₙ—CO—NH₂, (CH₂)_{q}—COOH, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (CH₂)ₙ—C(CH₃)₂—CO—NH₂, (CH₂)ₙ—C(CH₃)₂—COOH, where the alkyl and cycloalkyl radicals may be substituted by fluorine atoms and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, 2-cyanopyrrolidin-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholin-4-yl, thiomorpholine-1,1-dioxid-4-yl, NH—(CH₂)ₙ-aryl-(CH₂)ₙ-aryl, NH—(CH₂)ᵣ—OH, NH—CH(CH₂OH)₂, NH—C(CH₂OH)₃, N[(C₁-C₆)-alkyl-OH]₂, N[(C₁-C₆)-alkyl][(C₁-C₆)-alkyl-OH], D-glucamin-N-yl, N-methyl-D-glucamin-N-yl, NH—[(C₁-C₈)-alkyl]-CO—O(C₁-C₆)-alkyl, NH—[(C₁-C₈)-alkyl]-COOH, NH—[(C₁-C₈)-alkyl]-CONH₂, N[(C₁-C₆)-alkyl][(C₁-C₈)-alkyl]-CO—O(C₁-C₆)-alkyl, N[(C₁-C₆)-alkyl][(C₁-C₈)-alkyl]-COOH, N[(C₁-C₆)-alkyl][(C₁-C₈)-alkyl]-CONH₂, NH—[C(H)(aryl)]-CO—O(C₁-C₆)-alkyl, NH—[C(H)(aryl)]-COOH, NH—[C(H)(aryl)]-CONH₂, N[(C₁-C₆)-alkyl][C(H)(aryl)]-CO—O(C₁-C₆)-alkyl, N[(C₁-C₆)-alkyl][C(H)(aryl)]-COOH, N[(C₁-C₆)-alkyl][C(H)(aryl)]-CONH₂, NH—[C(H)(heteroaryl)]-CO—O(C₁-C₆)-alkyl, NH—[C(H)(heteroaryl)]-COOH, NH—[C(H)(heteroaryl)]-CONH₂, N[(C₁-C₆)-alkyl][C(H)(heteroaryl)]-CO—O(C₁-C₆)-alkyl, N[(C₁-C₆)-alkyl][C(H)(heteroaryl)]-COOH, N[(C₁-C₆)-alkyl][C(H)(heteroaryl)]-CONH₂, N[(C₁-C₆)-alkyl][(C₃-C₈)-cycloalkyl]-CO—O(C₁-C₆)-alkyl, N[(C₁-C₆)-alkyl][(C₃-C₈)-cycloalkyl]-COOH, N[(C₁-C₆)-alkyl][(C₃-C₈)-cycloalkyl]-CONH₂, NH—[(C₃-C₈)-cycloalkyl]-CO—O(C₁-C₆)-alkyl, NH—[(C₃-C₈)-cycloalkyl]-COOH, NH—[(C₃-C₈)-cycloalkyl]-CONH₂, NH—(CH₂)ᵣ—SO2-(C₁-C₆)-alkyl, NH—[(C₁-C₆)-alkyl]-SO₃H, NH—[(C₁-C₆)-alkyl]-SO₂—NH₂, N[(C₁-C₆)-alkyl]{[(C₁-C₆)-alkyl]-SO₃H},
where the alcohol (OH) or ketone (C═O) functions may be replaced by F or CF₂;
R18 is (C₁-C₈)-alkyl, (C₃-C₈)-cycloalkyl, (CH₂)_{q}—[(C₃-C₈)-cycloalkyl], (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₆)-alkyl, O—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
R20 is H, (C₁-C₆)-alkyl, (C₃-C₈)-cycloalkyl, aryl, [(C₁-C₆)-alkyl]-aryl;
R21 is H, F, CF₃, (C₁-C₆)-alkyl, (C₃-C₈)-cycloalkyl, OH, O—(C₁-C₆)-alkyl, O—(C₃-C₈)-cycloalkyl, O—(CH₂)ₙ-aryl, O—(CO)—(C₁-C₆)-alkyl, O—(CO)—(C₃-C₈)-cycloalkyl, O—(CO)—O—(C₁-C₆)-alkyl, O—(CO)—O—(C₃-C₈)-cycloalkyl, NH—[(C₁-C₆)-alkyl]-aryl, NH₂, NH—(C₁-C₆)-alkyl, NH—(CO)—(C₁-C₆)-alkyl;
R22 is H, CF₃, (C₁-C₆)-alkyl, aryl, [(C₁-C₆)-alkyl]-aryl;
and physiologically compatible salts thereof.

2. The compound of claim 1, wherein,
R, R′ are each independently H, (CH₂)ₙ-aryl, (C₁-C₆)-alkyl, where (C₁-C₆)-alkyl or the aryl radical may be substituted by halogen,
or R and R′ together form a ring having from three to eight carbon atoms, where one carbon atom may be replaced by O, S(O)ₘ, NR13 or NR15;
m is 0, 1, 2;
n is 0, 1, 2, 3;
p is 1, 2, 3, 4;
q is 1, 2, 3;
r is 2, 3, 4, 5;
v is 0, 1, 2, 3;
A, D, E, G, L are each independently C or N, where there is no corresponding R1, R2, R3, R4, R5 substituent when they are defined as N, or R2-D=E-R3 or R4-G=L-R5 are defined as S or O;
R1, R2, R3, R4, R5 are each independently H, F, Cl, Br, I, CN, CF₃, (C₁-C₆)-alkyl, (C₃-C₆)-cycloalkyl, (CH₂)ₙ-[(C₃-C₆)-cycloalkyl], (CH₂)ₙ-[(C₇-C₁₀)-bicycloalkyl], (CH₂)ₙ-[(C₇-C₁₂)-tricycloalkyl], adamantan-1-yl, adamantan-2-yl, (CH₂)ₙ-aryl, (CH₂)ₙ-heteroaryl, OCF₃, O-R11, NR13R15, NH—CN, S(O)ₘ-R12, SO₂—NH₂, SO₂—N═CH—N(CH₃)₂, SO₂—NH—CO-R12, SO₂—NH—CONHR12, SO₂—NH—CO—R16, SO₂—NH—[(C₁-C₆)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₆)-alkyl]₂, SO₂-R16, SF₅, CO—O[(C₁-C₆)-alkyl], CO—O[(C₃-C₆)-cycloalkyl], CO—O—(CH₂)ₙ-aryl, CO—O—(CH₂)ₙ-heteroaryl, CO—NH₂, CO—NH—CN, CO—NH—[(C₁-C₆)-alkyl], CO—N[(C₁-C₆)-alkyl]₂, CO—NH—[(C₃-C₆)-cycloalkyl], C(═NH)—O—[(C₁-C₆-alkyl)], C(═NH)—NH₂, C(═NH)—NR12R13, C(═NH)—R16, C(═NR13)-NR12R13, (CH₂)ₙ—C(═NSO₂-R12)NH₂, CO—NH—SO₂-R16, CO—NH—SO₂—NHR12, CO-R16, COOH, CO—(C₁-C₆)-alkyl, CO—(C₃-C₆)-cycloalkyl, CO-aryl, CO-heteroaryl, CH(OH)-aryl, CH(OH)-heteroaryl, CH[O—(C₁-C₄)-alkyl]-aryl, CH[O—(C₁-C₄)-alkyl]-heteroaryl, CHF-aryl, CHF-heteroaryl, CF₂-aryl, CF₂-heteroaryl, CHO, CH₂—OH, CH₂—CN, CH₂—O-R12, CH₂—O—(CH₂)_{q}—COOH,
where the alkyl, cycloalkyl, cycloalkenyl, bicycloalkyl, bicycloalkenyl and tricycloalkyl radicals may be substituted by fluorine atoms and where the aryl or heteroaryl radicals may be substituted by halogen, CN, (C₁-C₄)-alkyl, (C₃-C₆)-cycloalkyl, O—(C₁-C₄)-alkyl, (CH₂)ₙ-aryl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl and where the alkyl radicals may be substituted by fluorine atoms;
where the R1 and R2, R2 and R3, R3 and R4 or R4 and R5 radicals may in each case together be defined as —(CH₂)₃— or —(CH₂)₄— or —CH═CH—CH═CH—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or the X-bicyclic heterocycle may be unsubstituted or mono- or polysubstituted by
R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ₙ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C1-C8)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ-R12, SO₂-R16, SO₂—N═CH—N(CH₃)₂, 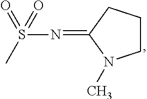
SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—-[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl], (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkenyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂, 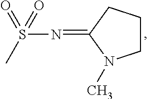
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as X-bicyclic heterocycle, X-aryl or X-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ,
R11 is H, (C₁-C₈)-alkyl, (C₂-C₆)-alkenyl, (C₂-C₆)-alkynyl, (C₃-C₆)-cycloalkyl, (CH₂)_{q}-[(C₃-C₆)-cycloalkyl], (CH₂)ₙ-[(C₇-C₁₂)-bicycloalkyl], (CH₂)ₙ-[(C₇-C₁₂)-tricycloalkyl], (CH₂)ₙ-aryl, (CH₂)ₙ—CO—[O—(C₁-C₆)-alkyl], (CH₂)ₙ—CO—[O—(C₃-C₆)-cycloalkyl], (CH₂)ₙ—CO—[(C₁-C₆)-alkyl], (CH₂)ₙ—CO—[(C₃-C₆)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)_{q}—CO—NH—CN, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₆)-alkyl], (CH₂)ₙ—CO—N[(C₁-C₆)-alkyl]₂, (CH₂)ₙ—CO—NH—[(C₃-C₆)-cycloalkyl], (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21 (CONH₂)₂, (CH₂)ₙ—CR21 (COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₈)-alkyl], (CH₂)ₙ—C(CH₃)₂—CO—O[(C₃-C₈)-cycloalkyl], (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₆)-alkyl], (CH₂)ₙ—C(CH₃)₂—CO—N[(C₁-C₆)-alkyl]₂, (CH₂)ₙ—C(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₆)-alkyl], (CH₂)ₙ—CO—NH—C(CH₃)₂—CONH₂, (CH₂)ₙ—CO—NH—C(CH₃)₂—COOH,
where the alkyl, alkenyl, alkynyl and cycloalkyl and bicycloalkyl radicals may be substituted by fluorine atoms
and where the aryl or heteroaryl radical may be substituted by halogen, CN, (C₁-C₄)-alkyl, (C₃-C₆)-cycloalkyl, O—(C₁-C₄)-alkyl, S(O)ₘ—(C₁-C₄)-alkyl, SO₂—NH₂, COOH, CONH₂, CO—O(C₁-C₆)-alkyl, CO—(C₁-C₆)-alkyl, and
where the alkyl radicals may be substituted by fluorine atoms;
R12 is H, (C₁-C₆)-alkyl, (C₃-C₆)-cycloalkyl, (CH₂)_{q}-[(C₃-C₆)-cycloalkyl], (CH₂)ₙ-aryl, (CH₂)ₙ-heteroaryl,
where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, CO—(C₁-C₄)-alkyl and where the alkyl radicals may be substituted by fluorine atoms;
R13 is H, SO₂—[(C₁-C₆)-alkyl], SO₂—[(C₃-C₆)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂,
where the alkyl and cycloalkyl radicals may be substituted by fluorine atoms and where the aryl or heteroaryl radical may be substituted by halogen, CN, CF₃, (C₁-C₄)-alkyl, (C₃-C₆)-cycloalkyl, O—[(C₁-C₄)-alkyl], S(O)ₘ-[(C₁-C₄)-alkyl], SO₂—NH₂, COOH, CONH₂, CO—[O(C₁-C₄)-alkyl], CO—(C₁-C₃)-alkyl, and where the alkyl radicals may be substituted by fluorine atoms;
R15 is H, (C₁-C₈)-alkyl,
where the alkyl radical may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholin-4-yl, thiomorpholine-1,1-dioxid-4-yl, NH—(CH₂)ₙ-aryl-(CH₂)ₙ-aryl, NH—(CH₂)ᵣ—OH, NH—CH(CH₂OH)₂, NH—C(CH₂OH)₃, N[(C₁-C₄)-alkyl-OH]₂, D-glucamin-N-yl, N-methyl-D-glucamin-N-yl, NH—[(C₁-C₆)-alkyl]-CO—O(C₁-C₄)-alkyl, NH—[(C₁-C₄)-alkyl]-COOH, NH—[(C₁-C₄)-alkyl]-CONH₂, N[(C₁-C₆)-alkyl][(C₁-C₈)-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(CH₂)ᵣ—SO2-(C₁-C₄)-alkyl, NH—[(C₁-C₄)-alkyl]-SO₃H, NH—[(C₁-C₄)-alkyl]-SO₂—NH₂, where the alcohol (OH) or ketone (C═O) functions may be replaced by F or CF₂;
R18 is (C₁-C₆)-alkyl, (C₃-C₆)-cycloalkyl, (CH₂)ₙ-[(C₃-C₆)-cycloalkyl], (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₄)-alkyl, O—(C₁-C₄)-alkyl, SO₂—NH₂, COOH, CONH₂, CO—[O(C₁-C₄)-alkyl], CO—(C₁-C₄)-alkyl, and where the alkyl radicals may be substituted by fluorine atoms;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
and the physiologically compatible salts thereof.

3. The compound of claim 2, wherein.
R, R′ are each independently H, (CH₂)ₙ-aryl (C₁-C₄)-alkyl where (C₁-C₄)-alkyl or the aryl radical may be substituted by halogen
or R and R′ together form a ring having from three to eight carbon atoms where one carbon atom may be replaced by O, S(O)ₘ, NR13 or NR15;
m is 0, 1, 2;
n is 0, 1, 2;
p is 1, 2, 3;
q is 1, 2, 3;
r is 2, 3, 4;
v is 0, 1, 2;
A, D, E, G, L are each independently C or N, where there is no corresponding R1, R2, R3, R4, R5 substituents when they are defined as N, or R2-D=E-R3 or R4-G=L-R5 are defined as S or O;
R1, R2, R3, R4, R5 are each independently H, F, Cl, Br, I, CN, CF₃, (C₁-C₄)-alkyl, (C₃-C₆)-cycloalkyl, adamantan-1-yl, adamantan-2-yl, (CH₂)ₙ-aryl, (CH₂)ₙ-heteroaryl, OCF₃, O-R11, NR13R15, S(O)ₘ-R12, SO₂—NH₂, SO₂—N═CH—N(CH₃)₂, SO₂—NH—CO-R12, SO₂—NH—CO—NHR12, SO₂—NH—CO-R16, SO₂—NH—[(C₁-C₄)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₄)-alkyl]₂, SO₂-R16, SF₅, CO—O[(C₁-C₄)-alkyl], CO—O[(C₃-C₄)-cycloalkyl], CO—NH₂, CO—NH—[(C₁-C₄)-alkyl], CO—N[(C₁-C₄)-alkyl]₂, CO—NH—[(C₃-C₆)-cycloalkyl], C(═NH)—O—[(C₁-C₄-alkyl)], C(═NH)—NH₂, C(═NH)—NR12R13, C(═NH)—R16, C(═NR13)-NR12R13, (CH₂)ₙ—C(═NSO₂-R12)NH₂, CO—NH—SO₂-R16, CO—NH—SO₂—NHR12, CO-R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-Aryl, CF₂-heteroaryl, CH₂—OH, CH₂—CN, CH₂—O-R12, CH₂—O—(CH₂)_{q}—COOH,
where the alkyl and cycloalkyl radicals may be substituted by fluorine atoms and where the aryl or heteroaryl radicals may be substituted by halogen, CN, (C₁-C₄)-alkyl, (C₃-C₆)-cycloalkyl, O—(C₁-C₄)-alkyl, (CH₂)ₙ-aryl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl, and where the alkyl radicals may be substituted by fluorine atoms;
where the R1 and R2, R2 and R3, R3 and R4 or R4 and R5 radicals in each case together be defined as —(CH₂)₃— or —(CH₂)₄— or —CH═CH—CH═CH—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or the X-bicyclic heterocycle may be unsubstituted or mono- or polysubstituted by
R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ᵥ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, 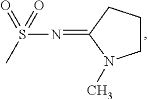
S(O)ₘ-R12, SO₂-R16, SO₂—N═CH—N(CH₃)₂, SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—-[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl], (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkenyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ, —O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂, 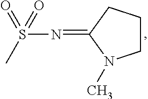
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as X-bicyclic heterocycle, X-aryl or X-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)m
R11 is H, (C₁-C₈)-alkyl, (C₂-C₆)-alkynyl, (C₃-C₆)-cycloalkyl, (CH₂)ₙ-aryl, (CH₂)ₙ—CO—[(C₁-C₄)-alkyl], (CH₂)ₙ—CO—[O—(C₃-C₆)-cycloalkyl], (CH₂)ᵣ—CO—[(C₁-C₄)-alkyl], (CH₂)ₙ—CO—[(C₃-C₆)-cycloalkyl], (CH₂)ₙ—CO-aryl, (CH₂)ₙ—CO—-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkenyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₄)]-alkyl, (CH₂)ₙ—C(CH₃)₂—CO—O[(C₃-C₆)]-cycloalkyl, (CH₂)ₙ—C(CH₃)₂—CO—O—(CH₂)ₙ-aryl, (CH₂)ᵣ—C(CH₃)₂—CO—NH₂, (CH₂)ₙ—C(CH₃)₂—CO—NH—[(C₁-C₄)-alkyl], (CH₂)ₙ—C(CH₃)₂—CO—N[(C₁-C₄)-alkyl]₂, (CH₂)ₙ—(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₄)-alkyl], (CH₂)ₙ—CO—NH—C(CH₃)₂—CONH₂, (CH₂)ₙ—CO—NH—C(CH₃)₂—COOH,
where the alkyl, alkenyl, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₆)-alkyl, and where the alkyl radicals may be substituted by fluorine atoms;
R12 is H, (C₁-C₄)-alkyl, (C₃-C₆)-cycloalkyl, (CH₂)_{q}-[(C₃-C₆)-cycloalkyl], (CH₂)ₙ-aryl, (CH₂)ₙ-heteroaryl,
where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂,
where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ-[(C₁-C₄)-alkyl], SO₂—NH₂, COOH, CONH₂, CO—[O(C₁-C₄)-alkyl], and where the alkyl radicals may be substituted by fluorine atoms;
R15 is H, (C₁-C₈)-alkyl,
where the alkyl radical may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholine-1,1-dioxid-4-yl, NH—(CH₂)ᵣ—OH, NH—CH(CH₂OH)₂, NH—C(CH₂OH)₃, N[(C₁-C₄)-alkyl-OH]₂, NH—[(C₁-C₄)-alkyl]-COOH, NH—[(C₁-C₄)-alkyl]-CONH₂, N[(C₁-C₆)-alkyl][C₁-C₈-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(CH₂)ᵣ—SO₂—(C₁-C₄)-alkyl, NH—[(C₁-C₄)-alkyl]-SO₃H, NH—[(C₁-C₄)-alkyl]-SO₂—NH₂, where the alcohol (OH) or ketone (C═O) functions may be replaced by F or CF₂;
R18 is (C₁-C₄)-alkyl, (C₃-C₆)-cycloalkyl, (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₄)-alkyl, O—(C₁-C₄)-alkyl, SO₂—NH₂, COOH, CONH₂, CO—[O(C₁-C₄)-alkyl], and where the alkyl radicals may be substituted by fluorine atoms;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
and the physiologically compatible salts thereof.

4. The compound of claim 3, wherein,
R, R′ are each independently H, aryl, (C₁-C₄)-alkyl where (C₁-C₄)-alkyl or the aryl radical may be substituted by halogen;
or R and R′ together form a ring having from three to eight carbon atoms, where one carbon atom may be replaced by O, S(O)ₘ, NR13 or NR15;
m is 0, 1, 2;
n is 0, 1, 2;
p is 1, 2, 3;
q is 1, 2, 3;
r is 2, 3;
v is 0, 1, 2;
A, D, E, G, L are each independently C or N, where there is no corresponding R1, R2, R3, R4, R5 substituent when they are defined as N or R2-D=E-R3 or R4-G=L-R5 are defined as S or O;
R1, R2, R3, R4, R5 are each independently H, F, Cl, Br, I, CN, CF₃, (C₁-C₄)-alkyl, (C₃-C₆)-cycloalkyl, (CH₂)ₙ-aryl, (CH₂)ₙ-heteroaryl, OCF₃, O-R11, NR13R15, S(O)ₘ—R12, SO₂—NH₂, SO₂—NH—CO-R12, SO₂—NH—CO—NHR12, SO₂—NH—CO-R16, SO₂—NH—[(C₁-C₄)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₄)-alkyl]₂, SO₂-R16, SF₅, CO—O[(C₁-C₄)-alkyl], CO—O[(C₃-C₄)-cycloalkyl], CO—NH₂, CO—NH—[(C₁-C₄)-alkyl], CO—N[(C₁-C₄)-alkyl]₂, C(═NH)—NH₂, C(═NH)—NR12R13, C(═NH)—R16, (CH₂)ₙ—C(═NSO₂-R12)NH₂, CO—NH—SO₂-R16, CO—NH—SO₂—NHR12, CO-R16, COOH, CO—(C₁-C₄)-alkyl, CO—(C₃-C₆)-cycloalkyl, CO-Aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O-R12, CH₂—O—(CH₂)_{q}—COOH,
where the alkyl or cycloalkyl radicals may be substituted by fluorine atoms and where the aryl or heteroaryl radicals may be substituted by halogen, CN, (C₁-C₄)-alkyl, O—(C₁-C₄)-alkyl, (CH₂)ₙ-aryl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl, and where the alkyl radicals may be substituted by fluorine atoms; and
where the radicals R1 and R2, R2 and R3, R3 and R4 or R4 and R5 may be in each case together be defined as —(CH₂)₃— or —(CH₂)₄—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or X-bicyclic heterocycle may be unsubstituted or mono- or polysubstituted by
R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ₙ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C1-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, 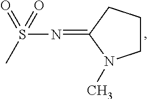
S(O)ₘ-R12, SO₂-R16, SO₂—N═CH—N(CH₃)₂, SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—-[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ₙ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ, —P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—N[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂, 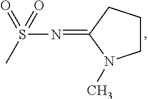
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as X-bicyclic heterocycle, X-aryl or X-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ
R11 is H, (C₁-C₈)-alkyl, (C₂-C₆)-alkynyl, (C₃-C₆)-cycloalkyl, (CH₂)ₙ-aryl, (CH₂)ₙ—CO—[O—(C₁-C₄)-alkyl], (CH₂)ₙ—CO—[O—(C₃-C₆)-cycloalkyl], (CH₂)ₙ—CO—[(C₁-C₄)-alkyl], (CH₂)ₙ—CO—[(C₃-C₆)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₄)]-alkyl, (CH₂)ₙ—C(CH₃)₂—CO—O[(C₃-C₆)]-cycloalkyl, (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₄)-alkyl], (CH₂)ₙ—C(CH₃)₂—CO—N[(C₁-C₄)-alkyl]₂, (CH₂)ₙ—(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₄)-alkyl], (CH₂)ₙ—CO—NH—C(CH₃)₂—CONH₂, (CH₂)ₙ—CO—NH—C(CH₃)₂—COOH,
where the alkyl, alkenyl, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₆)-alkyl, and where the alkyl radicals may be substituted by fluorine atoms;
R12 is H, (C₁-C₄)-alkyl, (C₃-C₆)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂,
where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ-[(C₁-C₄)-alkyl], SO₂—NH₂, COOH, CONH₂, CO—[O(C₁-C₄)-alkyl], and where the alkyl radicals may be substituted by fluorine atoms;
R15 is H, (C₁-C₈)-alkyl,
where the alkyl radical may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholine-1,1-dioxid-4-yl, NH—(CH₂)ᵣ—OH, NH—CH(CH₂OH)₂, NH—C(CH₂OH)₃, N[(C₁-C₄)-alkyl-OH]₂, NH—[(C₁-C₄)-alkyl]-COOH, NH—[(C₁-C₄)-alkyl]-CONH₂, N[(C₁-C₆)-alkyl][C₁-C₈-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(CH₂)ᵣ—SO₂—(C₁-C₄)-alkyl, NH—[(C₁-C₄)-alkyl]-SO₃H, NH—[(C₁-C₄)-alkyl]-SO₂—NH₂, where the alcohol (OH) or ketone (C═O) functions may be replaced by F or CF₂;
R18 is (C₁-C₄)-alkyl, (C₃-C₆)-cycloalkyl, (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₄)-alkyl, O—(C₁-C₄)-alkyl, SO₂—NH₂, COOH, CONH₂, CO—[O(C₁-C₄)-alkyl], and where the alkyl radicals may be substituted by fluorine atoms;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
and the physiologically compatible salts thereof.

5. A compound of the formula I 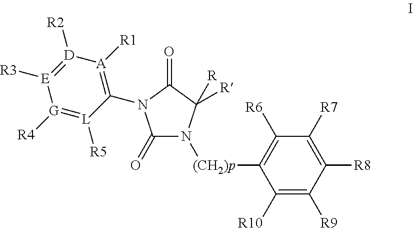
in which
m is 0, 1, 2;
n is 0, 1, 2, 3, 4;
p is 1, 2, 3, 4, 5;
q is 1, 2, 3, 4;
r is 2, 3, 4, 5, 6;
v is 0, 1, 2, 3, 4;
A, D, E, G, L are each independently C or N, where there is no corresponding R1, R2, R3, R4, R5 substituent when they are defined as N, or R2-D=E-R3 or R4-G=L-R5 are defined as S or O;
R1, R2, R3, R4, R5 are each independently H, F, Cl, Br, I, CN, N₃, NC, NO₂, CF₃, (C₁-C₈)-alkyl, (C₃-C₈)-cycloalkyl, (CH₂)_{q}-[(C₃-C₈)-cycloalkyl], (CH₂)ₙ-[(C₃-C₈)-cycloalkenyl], (CH₂)ₙ-[(C₇-C₁₂)-bicycloalkyl], (CH₂)ₙ-[(C₇-C₁₂)-bicycloalkenyl], (CH₂)ₙ-[(C₇-C₁₂)-tricycloalkyl], adamantan-1-yl, adamantan-2-yl, (CH₂)ₙ-aryl, (CH₂)ₙ-heteroaryl, OCF₃, O-R11, NR13R15, NH—CN, S(O)ₘ-R12, SO₂—NH₂, SO₂—N═CH—N(CH₃)₂, SO₂—NH—CO-R12, SO₂—NH—CO—NHR12, SO₂—NH—CO-R16, SO₂—NH—[(C₁-C₈)-alkyl], SO₂—NH—[(C₃-C₈)-cycloalkyl], SO₂—NH—(CH₂)ᵣ—OH, SO₂—NH—(CH₂)ₙ-aryl, SO₂—NH—(CH₂)ₙ-heteroaryl, SO₂—N[(C₁-C₈)-alkyl]₂, SO₂—N[(CH₂)ₙ-aryl][(CH₂)ₙ-heteroaryl], SO₂-R16, SF₅, CO—O[(C₁-C₈)-alkyl], CO—O[(C₃-C₈)-cycloalkyl], CO—O—(CH₂)ᵣ—NH₂, CO—O—(CH₂)ₙ-aryl, CO—O—(CH₂)ₙ-heteroaryl, CO—NH₂, CO—NH—CN, CO—NH—[(C₁-C₈)-alkyl], CO—NH—(CH₂)ᵣ—OH, CO—N[(C₁-C₈)-alkyl]₂, CO—NH—[(C₃-C₈)-cycloalkyl], CO—N[(C₃-C₈)-cycloalkyl]₂, C(═NH)—O—[(C₁-C₆-alkyl)], C(═NH)—NH₂, C(═NH)—NR12R13, C(═NH)—R16, C(═NR13)-NR12R13, (CH₂)ₙ—C(═NSO₂-R12)NH₂, CO—NH—SO₂-R16, CO—NH—SO₂—NHR12, CO-R16, COOH, CO—(C₁-C₈)-alkyl, CO—(C₃-C₈)-cycloalkyl, CO—(CH₂)ₙ-[(C₇-C₁₂)-bicycloalkyl], CO—(CH₂)ₙ-[(C₇-C₁₂)-tricycloalkyl], CO-aryl, CO-heteroaryl, CH(OH)-aryl, CH(OH)-heteroaryl, CH[O—(C₁-C₆)-alkyl]-aryl, CH[O—(C₁-C₆)-alkyl]-heteroaryl, CHF-aryl, CHF-heteroaryl, CF₂-aryl, CF₂-heteroaryl, CHO, CH₂—OH, CH₂—CN, CH₂—O-R12, CH₂—O—(CH₂)ₙ—CO—O[(C₁-C₈)-alkyl], CH₂—O—(CH₂)ₙ—CO—NH₂, CH₂—O—(CH₂)_{q}—COOH,
where the alkyl, cycloalkyl, cycloalkenyl, bicycloalkyl, bicycloalkenyl and tricycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, (CH₂)ₙ-aryl, O—(CH₂)ₙ-aryl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms; and
where the R1 and R2, R2 and R3, R3 and R4 or R4 and F5 radicals may in each case together be defined as —(CH₂)₃— or —(CH₂)₄— or —CH═CH—CH═CH—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or the X-bicyclic heterocycle may be unsubstituted or mono- or polysubstituted by
R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ₙ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ-R12, SO₂-R16, SO₂—N═CH—N(CH₃)₂, 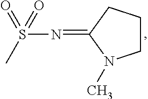
SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl], (CH₂)ₙ-—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—-N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂, 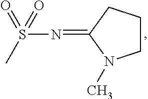
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as X-bicyclic heterocycle, X-aryl or X-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ
R11 is H, (C₁-C₈)-alkyl, (C₂-C₁₀)-alkenyl, (C₂-C₁₀)-alkynyl, (C₃-C₈)-cycloalkyl, (CH₂)_{q}-[(C₃-C₈)-cycloalkyl], (CH₂)ₙ-[(C₇-C₁₂)-bicycloalkyl], (CH₂)ₙ-[(C₃-C₁₀)-cycloalkenyl], (CH₂)ₙ-[(C₃-C₁₀)-bicycloalkenyl], (CH₂)ₙ—[(C₇-C₁₂)-tricycloalkyl], (CH₂)ₙ-aryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)_{q}—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (CH₂)ₙ—CO—N[(C₃-C₈)-cycloalkyl]₂, (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CR21[(CO—O(C₁-C₆)-alkyl)]₂, (CH₂)ₙ—CR21 (CONH₂)₂, (CH₂)ₙ—CR21 (COOH)₂, (CH₂)ₙ—CR21R22CO—O[(C₁-C₆)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₈)-alkyl], (CH₂)ₙ—C(CH₃)₂—CO—O[(C₃-C₈)-cycloalkyl], (CH₂)ₙ—C(CH₃)₂—CO—O—(CH₂)ᵣ—NH₂, (CH₂)ₙ—C(CH₃)₂—CO—O—(CH₂)ₙ-aryl, (CH₂)ₙ—C(CH₃)₂—CO—O—(CH₂)ₙ-heteroaryl, (CH₂)ₙ—C(CH₃)₂—CO—NH₂, (CH₂)ₙ—C(CH₃)₂—CO—NH—[(C₁-C₈)-alkyl], (CH₂)ₙ—C(CH₃)₂—CO—NH—(CH₂)ᵣ—OH, (CH₂)ₙ—C(CH₃)₂—CO—N[(C₁-C₈)-alkyl]₂, (CH₂)ₙ—C(CH₃)₂—CO—NH—[(C₃-C₈)-cycloalkyl], (CH₂)ₙ—C(CH₃)₂—CO—N[(C₃-C₈)-cycloalkyl]₂, (CH₂)ₙ—C(CH₃)₂—COOH, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl, bicycloalkyl, cycloalkenyl and bicycloalkenyl radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R12 is H, (C₁-C₈)-alkyl, (C₃-C₈)-cycloalkyl, (CH₂)_{q}-[(C₃-C₈)-cycloalkyl], (CH₂)ₙ—[(C₇-C₁₂)-bicycloalkyl], (CH₂)ₙ-[(C₇-C₁₂)-tricycloalkyl], (CH₂)ₙ-aryl, (CH₂)ₙ-heteroaryl, where the alkyl or cycloalkyl radicals may be substituted by fluorine atoms,
and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R13 is H, SO₂-[(C₁-C₈)-alkyl], SO₂-[(C₃-C₈)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂,
where the alkyl and cycloalkyl radicals may be substituted by fluorine atoms and where the aryl or heteroaryl radical may be substituted by halogen, CN, CF₃, (C₁-C₆)-alkyl, (C₃-C₆)-cycloalkyl, O—[(C₁-C₆)-alkyl], S(O)ₘ-[(C₁-C₆)-alkyl], SO₂—NH₂, COOH, CONH₂, CO—[O(C₁-C₆)-alkyl], CO—(C₁-C₆)-alkyl and where the alkyl radicals may be substituted by fluorine atoms;
R14 is H, (C₁-C₈)-alkyl, (C₃-C₈)-cycloalkyl, (CH₂)_{q}—[(C₃-C₈)-cycloalkyl], (CH₂)ₙ-aryl, (CH₂)ₙ-heteroaryl, (CH₂)ₙ—CO—[O—(C₁-C₈)-alkyl], (CH₂)ₙ—CO—[O—(C₃-C₈)-cycloalkyl], (CH₂)ₙ—CO—[O—(CH₂)ₙ-aryl], (CH₂)ₙ—CO—[O—(CH₂)ₙ-heteroaryl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (CH₂)ₙ—CO—N[(C₃-C₈)-cycloalkyl]₂, (CH₂)ₙ—C(CH₃)₂—CO—O[(C₁-C₈)]-alkyl, (CH₂)ₙ—C(CH₃)₂—CO—O[(C₃-C₈)]-cycloalkyl, (CH₂)ₙ—C(CH₃)₂—CO—O—(CH₂)ᵣ NH₂, (CH₂)ₙ—C(CH₃)₂—CO—NH₂, (CH₂)ₙ—C(CH₃)₂—CO—NH—(CH₂)ᵣ—OH, (CH₂)ₙ—C(CH₃)₂—COOH, where the alkyl and cycloalkyl radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R15 is H, (C₁-C₈)-alkyl, (C₃-C₈)-cycloalkyl, (CH₂)ₙ-aryl, (CH₂)ₙ-heteroaryl, (CH₂)ₙ—CO—[O—(C₁-C₈)-alkyl], (CH₂)ₙ—CO—[O—(C₃-C₈)-cycloalkyl], (CH₂)ₙ—CO—[O—(CH₂)ₙ-aryl], (CH₂)ₙ—CO—[O—(CH₂)ₙ-heteroaryl], CO—[(C₁-C₈)-alkyl], CO—[(C₃-C₈)-cycloalkyl], CO-aryl, CO-heteroaryl, (CH₂)ₙ—CO—NH₂, (CH₂)_{q}—COOH, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (CH₂)ₙ—C(CH₃)₂—CO—NH₂, (CH₂)ₙ—C(CH₃)₂—COOH, where the alkyl and cycloalkyl radicals may be substituted by fluorine atoms and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, 2-cyanopyrrolidin-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholin-4-yl, thiomorpholine-1,1-dioxid-4-yl, NH—(CH₂)ₙ-aryl-(CH₂)ₙ-aryl, NH—(CH₂)ᵣ—OH, NH—CH(CH₂OH)₂, NH—C(CH₂OH)₃, N[(C₁-C₆)-alkyl-OH]₂, N[(C₁-C₆)-alkyl][(C₁-C₆)-alkyl-OH], D-glucamin-N-yl, N-methyl-D-glucamin-N-yl, NH—[(C₁-C₈)-alkyl]-CO—O(C₁-C₆)-alkyl, NH—[(C₁-C₈)-alkyl]-COOH, NH—[(C₁-C₈)-alkyl]-CONH₂, N[(C₁-C₆)-alkyl][(C₁-C₈)-alkyl]-CO—O(C₁-C₆)-alkyl, N[(C₁-C₆)-alkyl][(C₁-C₈)-alkyl]-COOH, N[(C₁-C₆)-alkyl][(C₁-C₈)-alkyl]-CONH₂, NH—[C(H)(aryl)]-CO—O(C₁-C₆)-alkyl, NH—[C(H)(aryl)]-COOH, NH—[C(H)(aryl)]-CONH₂, N[(C₁-C₆)-alkyl][C(H)(aryl)]-CO—O(C₁-C₆)-alkyl, N[(C₁-C₆)-alkyl][C(H)(aryl)]-COOH, N[(C₁-C₆)-alkyl][C(H)(aryl)]-CONH₂, NH—[C(H)(heteroaryl)]-CO—O(C₁-C₆)-alkyl, NH—[C(H)(heteroaryl)]-COOH, NH—[C(H)(heteroaryl)]-CONH₂, N[(C₁-C₆)-alkyl][C(H)(heteroaryl)]-CO—O(C₁-C₆)-alkyl, N[(C₁-C₆)-alkyl][C(H)(heteroaryl)]-COOH, N[(C₁-C₆)-alkyl][C(H)(heteroaryl)]-CONH₂, N[(C₁-C₆)-alkyl][(C₃-C₈)-cycloalkyl]-CO—O(C₁-C₆)-alkyl, N[(C₁-C₆)-alkyl][(C₃-C₈)-cycloalkyl]-COOH, N[(C₁-C₆)-alkyl][(C₃-C₈)-cycloalkyl]-CONH₂, NH—[(C₃-C₈)-cycloalkyl]-CO—O(C₁-C₆)-alkyl, NH—[(C₃-C₈)-cycloalkyl]-COOH, NH—[(C₃-C₈)-cycloalkyl]-CONH₂, NH—(CH₂)ᵣ-SO2-(C₁-C₆)-alkyl, NH—[(C₁-C₆)-alkyl]-SO₃H, NH—[(C₁-C₆)-alkyl]-SO₂—NH₂, N[(C₁-C₆)-alkyl]{[(C₁-C₆)-alkyl]-SO₃H},
where the alcohol (OH) or ketone (C═O) functions may be replaced by F or CF₂;
R18 is (C₁-C₈)-alkyl, (C₃-C₈)-cycloalkyl, (CH₂)_{q}-[(C₃-C₈)-cycloalkyl], (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₆)-alkyl, O—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
R20 is H, (C₁-C₆)-alkyl, (C₃-C₈)-cycloalkyl, aryl, [(C₁-C₆)-alkyl]-aryl;
R21 is H, F, CF₃, (C₁-C₆)-alkyl, (C₃-C₈)-cycloalkyl, OH, O—(C₁-C₆)-alkyl, O—(C₃-C₈)-cycloalkyl, O—(CH₂)ₙ-aryl, O—(CO)—(C₁-C₆)-alkyl, O—(CO)—(C₃-C₈)-cycloalkyl, O—(CO)—O—(C₁-C₆)-alkyl, O—(CO)—O—(C₃-C₈)-cycloalkyl, NH—[(C₁-C₆)-alkyl]-aryl, NH₂, NH—(C₁-C₆)-alkyl, NH—(CO)—(C₁-C₆)-alkyl;
R22 is H, CF₃, (C₁-C₆)-alkyl, aryl, [(C₁-C₆)-alkyl]-aryl;
and physiologically compatible salts thereof.

6. The compound of claim 5, wherein,
m is 0, 1, 2;
n is 0, 1, 2, 3;
p is 1, 2, 3, 4;
q is 1, 2, 3;
r is 2, 3, 4, 5;
v is 0, 1, 2, 3;
A, D, E, G, L are each independently C or N, where there is no corresponding R1, R2, R3, R4, R5 substituent when they are defined as N, or R2-D=E-R3 or R4-G=L-R5 are defined as S or O;
R1, R2, R3, R4, R5 are each independently H, F, Cl, Br, I, CN, CF₃, (C₁-C₆)-alkyl, (C₃-C₆)-cycloalkyl, (CH₂)ₙ-[(C₃-C₆)-cycloalkyl], (CH₂)ₙ-[(C₇-C₁₀)-bicycloalkyl], (CH₂)ₙ-[(C₇-C₁₂)-tricycloalkyl], adamantan-1-yl, adamantan-2-yl, (CH₂)ₙ-aryl, (CH₂)ₙ-heteroaryl, OCF₃, O-R11, NR13R15, NH—CN, S(O)ₘ-R12, SO₂—NH₂, SO₂—N═CH—N(CH₃)₂, SO₂—NH—CO-R12, SO₂—NH—CONHR12, SO₂—NH—CO-R16, SO₂—NH—[(C₁-C₆)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₆)-alkyl]₂, SO₂-R16, SF₅, CO—O[(C₁-C₆)-alkyl], CO—O[(C₃-C₆)-cycloalkyl], CO—O—(CH₂)ₙ-aryl, CO—O—(CH₂)ₙ-heteroaryl, CO—NH—[(C₃-C₆)-cycloalkyl], C9=NH)—O—[(C₁-C₆-alkyl)], CO—NH—CN, CO—NH₂, CO—NH—[(C₁-C₆)-alkyl], CO—N[(C₁-C₆)-alkyl]₂, CO—NH—[(C₃-C₆)-cycloalkyl], C(═NH)—O—[(C₁-C₆-alkyl)], C(═NH)—NH₂, C(═NH)—NR12R13, (CH₂)ₙ—C(═NSO₂-R12)NH₂, C(═NH)—R16, C(═NR13)-NR12R13, CO—NH—SO₂-R16, CO—NH—SO₂—NHR12, CO-R16, COOH, CO—(C₁-C₆)-alkyl, CO—(C₃-C₆)-cycloalkyl, CO-aryl, CO-heteroaryl, CH(OH)-aryl, CH(OH)-heteroaryl, CH[O—(C₁-C₄)-alkyl]-aryl, CH[O—(C₁-C₄)-alkyl]-heteroaryl, CHF-aryl, CHF-heteroaryl, CF₂-aryl, CF₂-heteroaryl, CHO, CH₂—OH, CH₂—CN, CH₂—O-R12, CH₂—O—(CH₂)_{q}—COOH,
where the alkyl, cycloalkyl, cycloalkenyl, bicycloalkyl, bicycloalkenyl and tricycloalkyl radicals may be substituted by fluorine atoms and where the aryl or heteroaryl radicals may be substituted by halogen, CN, (C₁-C₄)-alkyl, (C₃-C₆)-cycloalkyl, O—(C₁-C₄)-alkyl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl and where the alkyl radicals may be substituted by fluorine atoms;
where the R1 and R2, R2 and R3, R3 and R4 or R4 and R5 radicals may in each case together be defined as —(CH₂)₃— or —(CH₂)₄— or —CH═CH—CH═CH—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or the X-bicyclic heterocycle may be unsubstituted or mono- or polysubstituted by
R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ᵥ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ-R12, SO₂-R16, SO₂—N═CH—N(CH₃)₂, 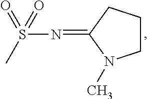
SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—-[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl], (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂, 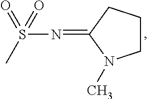
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as X-bicyclic heterocycle, X-aryl or X-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ,
R11 is H, (C₁-C₈)-alkyl, (C₂-C₆)-alkenyl, (C₂-C₆)-alkynyl, (C₃-C₆)-cycloalkyl, (CH₂)_{q}-[(C₃-C₆)-cycloalkyl], (CH₂)ₙ-[(C₇-C₁₂)-bicycloalkyl], (CH₂)ₙ-[(C₇-C₁₂)-tricycloalkyl], (CH₂)ₙ-aryl, (CH₂)ₙ—CO—[O—(C₁-C₆)-alkyl], (CH₂)ₙ—CO—[O—(C₃-C₆)-cycloalkyl], (CH₂)ₙ—CO—[(C₁-C₆)-alkyl], (CH₂)ₙ—CO—[(C₃-C₆)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)_{q}—CO—NH—CN, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₆)-alkyl], (CH₂)ₙ—CO—N[(C₁-C₆)-alkyl]₂, (CH₂)ₙ—CO—NH—[(C₃-C₆)-cycloalkyl], (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21 (CONH₂)₂, (CH₂)ₙ—CR21 (COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₈)-alkyl], (CH₂)ₙ—C(CH₃)₂—CO—O[(C₃-C₈)-cycloalkyl], (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₆)-alkyl], (CH₂)ₙ—C(CH₃)₂—CO—N[(C₁-C₆)-alkyl]₂, (CH₂)ₙ—C(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₆)-alkyl], (CH₂)ₙ—CO—NH—C(CH₃)₂—CONH₂, (CH₂)ₙ—CO—NH—C(CH₃)₂—COOH,
where the alkyl, alkenyl, alkynyl and cycloalkyl and bicycloalkyl radicals may be substituted by fluorine atoms
and where the aryl or heteroaryl radical may be substituted by halogen, CN, (C₁-C₄)-alkyl, (C₃-C₆)-cycloalkyl, O—(C₁-C₄)-alkyl, S(O)ₘ—(C₁-C₄)-alkyl, SO₂—NH₂, COOH, CONH₂, CO—O(C₁-C₆)-alkyl, CO—(C₁-C₆)-alkyl, and
where the alkyl radicals may be substituted by fluorine atoms;
R12 is H, (C₁-C₆)-alkyl, (C₃-C₆)-cycloalkyl, (CH₂)_{q}-[(C₃-C₆)-cycloalkyl], (CH₂)ₙ-aryl, (CH₂)ₙ-heteroaryl,
where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, CO—(C₁-C₄)-alkyl and where the alkyl radicals may be substituted by fluorine atoms;
R13 is H, SO₂—[(C₁-C₆)-alkyl], SO₂—[(C₃-C₆)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂, where the alkyl and cycloalkyl radicals may be substituted by fluorine atoms and where the aryl or heteroaryl radical may be substituted by halogen, CN, CF₃, (C₁-C₄)-alkyl, (C₃-C₆)-cycloalkyl, O—[(C₁-C₄)-alkyl], S(O)ₘ-[(C₁-C₄)-alkyl], SO₂—NH₂, COOH, CONH₂, CO—[O(C₁-C₄)-alkyl], CO—(C₁-C₄)-alkyl, and where the alkyl radicals may be substituted by fluorine atoms;
R15 is H, (C₁-C₈)-alkyl,
where the alkyl radical may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholin-4-yl, thiomorpholine-1,1-dioxid-4-yl, NH—(CH₂)ₙ-aryl-(CH₂)ₙ-aryl, NH—(CH₂)ᵣ—OH, NH—CH(CH₂OH)₂, NH—C(CH₂OH)₃, N[(C₁-C₄)-alkyl-OH]₂, D-glucamin-N-yl, N-methyl-D-glucamin-N-yl, NH—[(C₁-C₆)-alkyl]-CO—O(C₁-C₄)-alkyl, NH—[(C₁-C₄)-alkyl]-COOH, NH—[(C₁-C₄)-alkyl]-CONH₂, N[(C₁-C₆)-alkyl][(C₁-C₈)-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH—[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(CH₂)ᵣ—SO₂—(C₁-C₄)-alkyl, NH—[(C₁-C₄)-alkyl]-SO₃H, NH—[(C₁-C₄)-alkyl]-SO₂—NH₂, where the alcohol (OH) or ketone (C═O) functions may be replaced by F or CF₂;
R18 is (C₁-C₆)-alkyl, (C₃-C₆)-cycloalkyl, (CH₂)ₙ-[(C₃-C₆)-cycloalkyl], (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₄)-alkyl, O—(C₁-C₄)-alkyl, SO₂—NH₂, COOH, CONH₂, CO—[O(C₁-C₄)-alkyl], CO—(C₁-C₄)-alkyl, and where the alkyl radicals may be substituted by fluorine atoms;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
and the physiologically compatible salts thereof.

7. The compound of claim 6, wherein,
m is 0, 1, 2;
n is 0, 1, 2;
p is 1, 2, 3;
q is 1, 2, 3;
r is 2, 3, 4;
v is 0, 1, 2;
A, D, E, G, L are each independently C or N, where there is no corresponding R1, R2, R3, R4, R5 substituents when they are defined as N, or R2-D=E-R3 or R4-G=L-R5 are defined as S or O;
R1, R2, R3, R4, R5 are each independently H, F, Cl, Br, I, CN, CF₃, (C₁-C₄)-alkyl, (C₃-C₆)-cycloalkyl, adamantan-1-yl, adamantan-2-yl, (CH₂)ₙ-aryl, (CH₂)ₙ-heteroaryl, OCF₃, O-R11, NR13R15, S(O)ₘ-R12, SO₂—NH₂, SO₂—N═CH—N(CH₃)₂, SO₂—NH—CO-R12, SO₂—NH—CO—NHR12, SO₂—NH—CO-R16, SO₂—NH—[(C₁-C₄)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₄)-alkyl]₂, SO₂-R16, SF₅, CO—O[(C₁-C₄)-alkyl], CO—O[(C₃-C₄)-cycloalkyl], CO—NH₂, CO—NH—[(C₁-C₄)-alkyl], CO—N[(C₁-C₄)-alkyl]₂, CO—NH—[(C₃-C₆)-cycloalkyl], C(═NH)—O—[(C₁-C₄-alkyl)], C(═NH)—NH₂, C(═NH)—NR12R13, C(═NH)—R16, C(═NR13)-NR12R13, (CH₂)ₙ—C(═NSO₂-R12)NH₂, CO—NH—SO₂-R16, CO—NH—SO₂—NHR12, CO-R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-Aryl, CF₂-heteroaryl, CH₂—OH, CH₂—CN, CH₂—O-R12, CH₂—O—(CH₂)_{q}—COOH,
where the alkyl and cycloalkyl radicals may be substituted by fluorine atoms and where the aryl or heteroaryl radicals may be substituted by halogen, CN, (C₁-C₄)-alkyl, (C₃-C₆)-cycloalkyl, O—(C₁-C₄)-alkyl, (CH₂)ₙ-aryl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl, and where the alkyl radicals may be substituted by fluorine atoms;
and where the R1 and R2, R2 and R3, R3 and R4 or R4 and R5 radicals in each case together be defined as —(CH₂)₃— or —(CH₂)₄— or —CH═CH—CH═CH—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or the X-bicyclic heterocycle may be unsubstituted or mono- or polysubstituted by
R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ᵥ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ-R12, SO₂-R16, SO₂—N═CH—N(CH₃)₂, 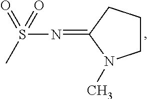
SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—-[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkenyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂, 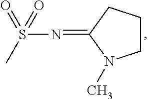
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as X-bicyclic heterocycle, X-aryl or X-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)m
R11 is H, (C₁-C₈)-alkyl, (C₂-C₆)-alkynyl, (C₃-C₆)-cycloalkyl, (CH₂)ₙ-aryl, (CH₂)ₙ—CO—[O—(C₁-C₄)-alkyl], (CH₂)ₙ—CO—[O—(C₃-C₆)-cycloalkyl], (CH₂)ₙ—CO—[(C₁-C₄)-alkyl], (CH₂)ₙ—CO—[(C₃-C₆)-cycloalkyl], (CH₂)ₙ—CO-aryl, (CH₂)ₙ—CO—-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkenyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₄)]-alkyl, (CH₂)ₙ—C(CH₃)₂—CO—O[(C₃-C₆)]-cycloalkyl, (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₄)-alkyl], (CH₂)ₙ—C(CH₃)₂—CO—-N[(C₁-C₄)-alkyl]₂, (CH₂)ₙ—(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₄)-alkyl], (CH₂)ₙ—CO—NH—C(CH₃)₂—CONH₂, (CH₂)ₙ—CO—NH—C(CH₃)₂—COOH,
where the alkyl, alkenyl, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₆)-alkyl, and where the alkyl radicals may be substituted by fluorine atoms;
R12 is H, (C₁-C₄)-alkyl, (C₃-C₆)-cycloalkyl, (CH₂)_{q}-[(C₃-C₆)-cycloalkyl], (CH₂)ₙ-aryl, (CH₂)ₙ-heteroaryl,
where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂,
where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ-[(C₁-C₄)-alkyl], SO₂—NH₂, COOH, CONH₂, CO—[O(C₁-C₄)-alkyl], and where the alkyl radicals may be substituted by fluorine atoms;
R15 is H, (C₁-C₈)-alkyl,
where the alkyl radical may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholine-1,1-dioxid-4-yl, NH—(CH₂)ᵣ—OH, NH—CH(CH₂OH)₂, NH—C(CH₂OH)₃, N[(C₁-C₄)-alkyl-OH]₂, NH—[(C₁-C₄)-alkyl]-COOH, NH—[(C₁-C₄)-alkyl]-CONH₂, N[(C₁-C₆)-alkyl][C₁-C₈-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(CH₂)ᵣ—SO₂—(C₁-C₄)-alkyl, NH—[(C₁-C₄)-alkyl]-SO₃H, NH—[(C₁-C₄)-alkyl]-SO₂—NH₂, where the alcohol (OH) or ketone (C═O) functions may be replaced by F or CF₂;
R18 is (C₁-C₄)-alkyl, (C₃-C₆)-cycloalkyl, (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₄)-alkyl, O—(C₁-C₄)-alkyl, SO₂—NH₂, COOH, CONH₂, CO—[O(C₁-C₄)-alkyl], and where the alkyl radicals may be substituted by fluorine atoms;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
and the physiologically compatible salts thereof.

8. The compound of claim 7, wherein,
m is 0, 1, 2;
n is 0, 1, 2;
p is 1, 2, 3;
q is 1, 2, 3;
r is 2, 3;
v is 0, 1, 2;
A, D, E, G, L are each independently C or N, where there is no corresponding R1, R2, R3, R4, R5 substituent when they are defined as N or R2-D=E-R3 or R4-G=L-R5 are defined as S or O;
R1, R2, R3, R4, R5 are each independently H, F, Cl, Br, I, CN, CF₃, (C₁-C₄)-alkyl, (C₃-C₆)-cycloalkyl, (CH₂)ₙ-aryl, (CH₂)ₙ-heteroaryl, OCF₃, O-R11, NR13R15, S(O)ₘ—R12, SO₂—NH₂, SO₂—NH—CO-R12, SO₂—NH—CO—NHR12, SO₂—NH—CO-R16, SO₂—NH—[(C₁-C₄)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₄)-alkyl]₂, SO₂-R16, SF₅, CO—O[(C₁-C₄)-alkyl], CO—O[(C₃-C₄)-cycloalkyl], CO—NH₂, CO—NH—[(C₁-C₄)-alkyl], CO—N[(C₁-C₄)-alkyl]₂, C(═NH)—NH₂, C(═NH)—NR12R13, C(═NH)—R16, (CH₂)ₙ—C(═NSO₂-R12)NH₂, CO—NH—SO₂-R16, CO—NH—SO₂—NHR12, CO-R16, COOH, CO—(C₁-C₄)-alkyl, CO—(C₃-C₆)-cycloalkyl, CO-Aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O-R12, CH₂—O—(CH₂)_{q}—COOH,
where the alkyl or cycloalkyl radicals may be substituted by fluorine atoms and where the aryl or heteroaryl radicals may be substituted by halogen, CN, (C₁-C₄)-alkyl, O—(C₁-C₄)-alkyl, (CH₂)ₙ-aryl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl, and where the alkyl radicals may be substituted by fluorine atoms; and
where the radicals R1 and R2, R2 and R3, R3 and R4 or R4 and R5 may in each case together be defined as —(CH₂)₃— or —(CH₂)₄—;
R6, R7, R8, R9, R10 are each independently X-bicyclic heterocycle, X-aryl or X-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the X-aryl or X-heteroaryl radical or X-bicyclic heterocycle may be unsubstituted or mono- or polysubstituted by
R11, F, Cl, Br, I, CN, N₃, NC, NO₂, CF₃, (CH₂)ₙ—O-R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O-R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂-R16, (CH₂)ₙ—NH—(CH₂)ₙ—SO₂-R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12-C(═NR13)-NHR12, (CH₂)ₙ—NR12-C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, 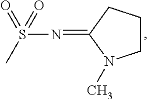
S(O)ₘ-R12, SO₂-R16, SO₂—N═CH—N(CH₃)₂, SO₂—NH—CO-R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—CHO, (CH₂)ₙ—C(═NH)—NH₂, (CH₂)ₙ—C(═NH)—NHOH, (CH₂)ₙ—C(═NH)-[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂-R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—-[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)—alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO-R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkenyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ₙ—NH₂, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂-R16, (CH₂)ₙ—NR12-CO—NH—(C₁-C₈)-alkyl, (CH₂)ₙ—NR12-CO—NH—(C₃-C₈)-cycloalkyl, (CH₂)ₙ—NR12-CO—NH₂, (CH₂)ₙ—NR12-CO—NH—SO₂—(C₁-C₈)-alkyl, (CH₂)ₙ—NR12-CO—NH—SO₂—(C₃-C₈)-cycloalkyl, (CH₂)ₙ—NR12-CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂-R16, SO₂—N═CH—N(CH₃)₂, 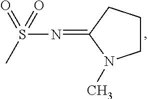
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as X-bicyclic heterocycle, X-aryl or X-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
X is O, S(O)ₘ
R11 is H, (C₁-C₈)-alkyl, (C₂-C₆)-alkynyl, (C₃-C₆)-cycloalkyl, (CH₂)ₙ-aryl, (CH₂)ₙ—CO—[O—(C₁-C₄)-alkyl], (CH₂)ₙ—CO—[O—(C₃-C₆)-cycloalkyl], (CH₂)ₙ—CO—[(C₁-C₄)-alkyl], (CH₂)ₙ—CO—[(C₃-C₆)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₄)]-alkyl, (CH₂)ₙ—C(CH₃)₂—CO—O[(C₃-C₆)]-cycloalkyl, (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₄)-alkyl], (CH₂)ₙ—C(CH₃)₂—CO—N[(C₁-C₄)-alkyl]₂, (CH₂)ₙ—(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₄)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₆)-alkyl, and where the alkyl radicals may be substituted by fluorine atoms;
R12 is H, (C₁-C₄)-alkyl, (C₃-C₆)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl, SO₂—(CH₂)ₙ—NH—R12, SO₂—(CH₂)ₙ—N(R12)₂,
where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ-[(C₁-C₄)-alkyl], SO₂—NH₂, COOH, CONH₂, CO—[O(C₁-C₄)-alkyl], and where the alkyl radicals may be substituted by fluorine atoms;
R15 is H, (C₁-C₈)-alkyl,
where the alkyl radical may be substituted by fluorine atoms;
R16 is aziridin-1-yl, azetidin-1-yl, 3-hydroxyazetidin-1-yl, piperidin-1-yl, 3-hydroxypiperidin-1-yl, 4-hydroxypiperidin-1-yl, 3-oxopiperidin-1-yl, 4-oxopiperidin-1-yl, pyrrolidin-1-yl, 3-pyrrolidinol-1-yl, morpholin-N-yl, piperazin-1-yl, 4-[(C₁-C₆)-alkyl]piperazin-1-yl, piperazin-2-on-1-yl, piperazin-2-on-4-yl, piperazine-2,3-dion-1-yl, piperazine-2,6-dion-1-yl, piperazine-2,6-dion-4-yl, thiomorpholine-1,1-dioxid-4-yl, NH—(CH₂)ᵣ—OH, NH—CH(CH₂OH)₂, NH—C(CH₂OH)₃, N[(C₁-C₄)-alkyl-OH]₂, NH—[(C₁-C₄)-alkyl]-COOH, NH—[(C₁-C₄)-alkyl]-CONH₂, N[(C₁-C₆)-alkyl][C₁-C₈-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(CH₂)ᵣ—SO₂—(C₁-C₄)-alkyl, NH—[(C₁-C₄)-alkyl]-SO₃H, NH—[(C₁-C₄)-alkyl]-SO₂—NH₂, where the alcohol (OH) or ketone (C═O) functions may be replaced by F or CF₂;
R18 is (C₁-C₄)-alkyl, (C₃-C₆)-cycloalkyl, (CH₂)ₙ-aryl, (CH₂)ₙ-heteroaryl, where the alkyl and cycloalkyl radicals may be substituted by fluorine atoms and where the aryl or heteroaryl radical may be substituted by halogen, CN, (C₁-C₄)-alkyl, O—(C₁-C₄)-alkyl, SO₂—NH₂, COOH, CONH₂, CO—[O(C₁-C₄)-alkyl], and where the alkyl radicals may be substituted by fluorine atoms;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
and the physiologically compatible salts thereof.

9. A compound of the formula Ia 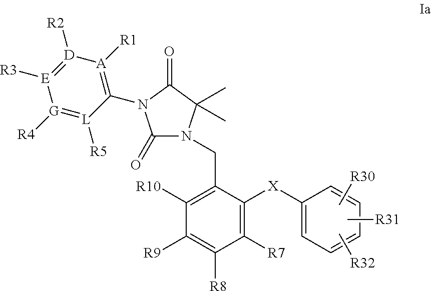
in which
m is 0, 1, 2;
n is 0, 1, 2;
q is 1, 2, 3;
r is 2, 3;
v is 0, 1, 2;
X is O, S(O)ₘ
A, D, E, G, L are each independently C or N, where there is no corresponding R1, R2, R3, R4, R5 substituent when they are defined as N, or R2-D=E=R3 or R4-G=L-R5 are defined as S or O;
R1, R2, R3, R4, R5 are each independently H, F, Cl, Br, I, CN, CF₃, (C₁-C₄)-alkyl, (C₃-C₆)-cycloalkyl, (CH₂)ₙ-aryl, (CH₂)ₙ-heteroaryl, OCF₃, O-R11, NR13R15, S(O)ₘ—R12, SO₂—NH₂, SO₂—NH—CO-R12, SO₂—NH—CO—NHR12, SO₂—NH—CO-R16, SO₂—NH—[(C₁-C₄)-alkyl], SO₂—NH—[(C₃-C₆)-cycloalkyl], SO₂—NH—(CH₂)ₙ-aryl, SO₂—NH—(CH₂)ₙ-heteroaryl, SO₂—N[(C₁-C₄)-alkyl]₂, SO₂-R16, SF₅, CO—O[(C₁-C₄)-alkyl], CO—O[(C₃-C₄)-cycloalkyl], CO—NH₂, CO—NH—[(C₁-C₄)-alkyl], CO—N[(C₁-C₄)-alkyl]₂, C(═NH)—NH₂, C(═NH)—NR12R13, C(═NH)—R16, (CH₂)ₙ—C(═NSO₂-R12)NH₂, CO—NH—SO₂-R16, CO—NH—SO₂—NHR12, CO-R16, COOH, CO—(C₁-C₄)-alkyl, CO—(C₃-C₆)-cycloalkyl, CO-Aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O-R12, CH₂—O—(CH₂)_{q}—COOH,
where the alkyl and cycloalkyl radicals may be substituted by fluorine atoms and where the aryl or heteroaryl radicals may be substituted by halogen, CN, (C₁-C₄)-alkyl, O—(C₁-C₄)-alkyl, (CH₂)ᵣ-aryl, O—(CH₂)ₙ-aryl, S(O)ₘ—(C₁-C₄)-alkyl, SO₂—NH₂, COOH, CONH₂, CO—O(C₁-C₄)-alkyl, CO—(C₁-C₄)-alkyl, and where the alkyl radicals may be substituted by fluorine atoms; and
where the R1 and R2, R2 and R3, R3 and R4 or R4 and R5 radicals may each together be defined as —(CH₂)₃— or —(CH₂)₄—;
R7, R8, R9, R10 are each independently H, F, Cl, Br, I, CN, CF₃, (C₁-C₄)-alkyl, (C₂-C₄)-alkynyl, (C₃-C₆)-cycloalkyl, aryl, heteroaryl, (CH₂)ₙ—CO—[O—(C₁-C₄)-alkyl], (CH₂)ₙ—CO—[(C₁-C₄)-alkyl], (CH₂)ₙ—CO—NH₂, (CH₂)ₙ—COOH, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (CH₂)ₙ—CO-R16, (CH₂)ₙ—OH, (CH₂)ₙ—O—(C₁-C₄)-alkyl, (CH₂)ₙ—O—(C₃-C₆)-cycloalkyl, (CH₂)ₙ—O—(CH₂)ₙ—CO—[O—(C₁-C₄)-alkyl], (CH₂)ₙ—O—(CH₂)ₙ—CO—[(C₁-C₄)-alkyl], (CH₂)ₙ—O—(CH₂)_{q}—COOH, (CH₂)ₙ—O—(CH₂)ₙ—P(O)(OH)[O—(C₁-C₄)-alkyl], (CH₂)ₙ—O—(CH₂)ₙ—P(O)[O—(C₁-C₄)-alkyl]₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₄)-alkyl], (CH₂)ₙ—O—(CH₂)ₙ—CR21R22-CO—O[(C₁-C₄)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO-R16, (CH₂)ₙ—O—(CH₂)ᵣ—OH, (CH₂)ₙ—O—(CH₂)ₙ—CO—NH—(CH₂)ᵣ—OH, O-R13, OCF₃, (CH₂)ₙ—NH₂, (CH₂)ₙ—NH—(C₁-C₄)-alkyl, (CH₂)ₙ—NH—(C₃-C₆)-cycloalkyl, (CH₂)ₙ—NH—(CH₂)ₙ—CO—[(C₁-C₄)-alkyl], (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O[(C₁-C₄)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO-R16, (CH₂)ₙ—NH—(CH₂)ₙ—SO₂—[(C₁-C₄)-alkyl], (CH₂)ₙ—NH—(CH₂)ₙ—SO₂—[(C₃-C₆)-cycloalkyl], (CH₂)ₙ—NH—SO₂—(CH₂)ₙ—NH—(C₁-C₄)-alkyl, (CH₂)ₙ—NH—SO₂—(CH₂)ₙ—NH—(C₃-C₆)-cycloalkyl, (CH₂)ₙ—NH—SO₂—(CH₂)ₙ—N[(C₁-C₄)-alkyl]₂, (CH₂)ₙ—NH—SO₂-R16, (CH₂)ₙ—NR12-CO—NH—(C₁-C₄)-alkyl, (CH₂)ₙ—NR12-CO—NH—(C₃-C₆)-cycloalkyl, (CH₂)ₙ—NR12-CO—NH₂, (CH₂)ₙ—NR12-CO—NH—SO₂—(C₁-C₄)-alkyl, (CH₂)ₙ—NH—CO—NH—(CH₂)ₙ—CO—[O—(C₁-C₄)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₄)-alkyl], (CH₂)ₙ—NH—C(═N—SO₂—(C₁-C₆)-alkyl)-NH₂, (CH₂)ₙ—NH—C(═N—SO₂—(C₁-C₄)-alkyl)-NH[(C₁-C₄)-alkyl], (CH₂)ₙ—NH—C(═N—SO₂—NH₂)—NH₂, (CH₂)ₙ—NH—C(═N—SO₂—NH₂)—NH[(C₁-C₄)-alkyl], (CH₂)ₙ—NH—C(═NH)—N[(C₁-C₄)-alkyl]₂, (CH₂)ₙ—NH—C(═N—SO₂—(C₁-C₄)-alkyl)-N[(C₁-C₄)-alkyl]₂, (CH₂)ₙ—NH—(CH₂)ₙ—CO—NH—[(C₁-C₄)-alkyl], (CH₂)ₙ—NH—(CH₂)ₙ—CO—NH—(CH₂)ᵣ—OH, (CH₂)ₙ—S(O)ₘ—(C₁-C₄)-alkyl, (CH₂)ₙ—S(O)ₘ—(C₃-C₆)-cycloalkyl, SO₂—N═CH—N(CH₃)₂, (CH₂)ₙ—SO₂—NH—CO—(C₁-C₄)-alkyl, (CH₂)ₙ—SO₂—NH—CO—(C₃-C₆)-cycloalkyl, (CH₂)ₙ—SO₂—NH—(C₁-C₄)-alkyl, (CH₂)ₙ—SO₂—NH—(C₃-C₆)-cycloalkyl, SO₂—NH—(CH₂)ᵣ—OH, SO₂—NH—(CH₂)ᵣ—NH₂, SF₅, (CH₂)_{q}—CN, (CH₂)ₙ—CO—NH—SO₂—NHR12, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13,
where the alkyl and cycloalkyl radicals may be substituted by fluorine atoms, and where the aryl or heteroaryl radicals may be substituted by halogen, CN, (C₁-C₄)-alkyl, (C₃-C₆)-cycloalkyl, O—(C₁-C₄)-alkyl, S(O)ₘ—(C₁-C₄)-Alkyl, SO₂⁻ NH₂, COOH, CONH₂, CO—[O(C₁-C₄)-alkyl], CO—(C₁-C₄)-alkyl, and where the alkyl radicals may be substituted by fluorine atoms;
R11 is H, (C₁-C₈)-alkyl, (C₂-C₆)-alkynyl, (C₃-C₆)-cycloalkyl, (CH₂)ₙ-aryl, (CH₂)ₙ—CO—[O—(C₁-C₄)-alkyl], (CH₂)ₙ—CO—[O—(C₃-C₆)-cycloalkyl], (CH₂)ₙ—CO—[(C₁-C₄)-alkyl], (CH₂)ₙ—CO—[(C₃-C₆)-cycloalkyl], (CH₂)ₙ—CO-aryl, (CH₂)ₙ—CO-heteroaryl, (CH₂)ₙ—CO—[O—(CH₂)ᵥ-aryl], (CH₂)ₙ—CO—[O—(CH₂)ᵥ-heteroaryl], (CH₂)_{q}—CO—NH₂, (CH₂)_{q}—COOH, (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₆)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22CO—O[(C₁-C₄)-alkyl], (CH₂)ₙ—CR21R22CONH₂, (CH₂)ₙ—CR21R22COOH, (CH₂)ₙ—CO-R16, (CH₂)ₙ—C(CH₃)₂—CO—O[(C₁-C₄)]-alkyl, (CH₂)ₙ—C(CH₃)₂—CO—O[(C₃-C₆)]-cycloalkyl, (CH₂)ₙ—C(CH₃)₂—CO—O—(CH₂)ₙ-aryl, (CH₂)ₙ—C(CH₃)₂—CO—NH₂, (CH₂)ₙ—C(CH₃)₂—CO—NH—[(C₁-C₄)-alkyl], (CH₂)ₙ—C(CH₃)₂—CO—N[(C₁-C₄)-alkyl]₂, (CH₂)ₙ—(CH₃)₂—CO—NH—[(C₃-C₆)-cycloalkyl], (CH₂)ₙ—C(CH₃)₂—COOH, (CH₂)ₙ—CO—NH—C(CH₃)₂—CO—O[(C₁-C₄)-alkyl], (CH₂)ₙ—CO—NH—C(CH₃)₂—CONH₂, (CH₂)ₙ—CO—NH—C(CH₃)₂—COOH,
where the alkyl, alkenyl, alkynyl and cycloalkyl radicals may be substituted by fluorine atoms, and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₆)-alkyl, and where the alkyl radicals may be substituted by fluorine atoms;
R30, R31, R32 are each independently R11, F, Cl, Br, I, CN, CF₃, (CH₂)ₙ—O-R11, O—R13, OCF₃, (CH₂)ₙ—NH—R11, (CH₂)ₙ—N[(CH₂)_{q}—CO—O(C₁-C₄)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—SO₂-R16, (CH₂)ₙ—NH—(CH₂)ₙ—SO₂—R12, (CH₂)ₙ—NR12-CO-R16, (CH₂)ₙ—NR12-CO—NR12R13, (CH₂)ₙ—NR12-CO—N(R12)₂, (CH₂)ₙ—NR12-CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NH—(CH₂)ₙ—CO—NH—[(C₁-C₄)-alkyl], (CH₂)ₙ—NH—(CH₂)ₙ—CO—N[(C₁-C₄)-alkyl]₂, (CH₂)ₙ—NH—C(CH₃)₂—CO—O(C₁-C₄)-alkyl, (CH₂)ₙ—NH—C(CH₃)₂—CO—O(C₃-C₆)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₄)-alkyl], (CH₂)ₙ—NH—C(CH₃)₂—CO—N[(C₁-C₄)-alkyl]₂, (CH₂)ₙ—NH—C(CH₃)₂—COOH, S(O)ₘ-R12, SO₂-R16, SO₂—N═CH—N(CH₃)₂, SO₂—NH—CO-R12, SO₂—NHR12, SO₂—N[(C₁-C₄)-alkyl]₂, SF₅, COOH, CO—NH₂, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂-R18, (CH₂)ₙ—C(═NH)—NHOH, (CH₂)ₙ—C(═NR13)NHR12, (CH₂)ₙ—C(═NR12)NR12R13, (CH₂)ₙ—C(═NSO₂-R12)NH₂,
where the alkyl and cycloalkyl radicals may be substituted by fluorine atoms, and where the aryl or heteroaryl radicals may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₄)-alkyl, and where the alkyl radicals may be substituted by fluorine atoms,
and where, when R3 is CN, NO₂ or halogen and R4 is CF₃ or halogen,
R30, R31 or R32 is a substituent other than hydrogen;
and the physiologically compatible salts thereof.

10. The compound of claim 9, wherein,
m is 0, 1, 2;
n is 0, 1, 2;
X is O, S(O)ₘ
A, D, E, G, L are each independently C or N, where the corresponding R1, R2, R3, R4, R5 substituent is absent when they are defined as N, or R4-G=L-R5 is defined as S;
R1, R2, R3, R4, R5 are each independently H, F, Cl, Br, I, CN, CF₃, (C₁-C₄)-alkyl, (CH₂)ₙ-aryl, —O—(CH₂)ₙ-aryl, OCF₃, O—(C₁-C₈)-alkyl, S(O)ₘ—(C₁-C₈)-alkyl, CO—O[(C₁-C₄)-alkyl], CO—(C₁-C₄)-alkyl, CO-Aryl, CH₂—CN;
where the alkyl radicals may be substituted by fluorine atoms;
R7, R8, R9, R10 are each H;
R30, R31, R32 are each independently H, (C₁-C₈)-alkyl, F, Cl, Br, —COOH, —COO(C₁-C₈)-alkyl, (CH₂)ₙ—CONH₂;
and where, when R3 is CN, NO₂ or halogen and R4 is CF₃ or halogen, one of the R30, R31 or R32 radicals is a substituent other than hydrogen;
and the physiologically compatible salts thereof.

11. A pharmaceutical composition comprising one or more compounds of claim 1.

12. A pharmaceutical composition comprising one or more compounds of claim 5.

13. A pharmaceutical composition comprising one or more compounds of claim 9.

14. The pharmaceutical composition of claim 11 and a pharmaceutically acceptable carrier.

15. The pharmaceutical composition of claim 12 and a pharmaceutically acceptable carrier.

16. The pharmaceutical composition of claim 13 and a pharmaceutically acceptable carrier.

17. The pharmaceutical composition of claim 14 and at least one further active ingredient.

18. The pharmaceutical composition of claim 15 and at least one further active ingredient.

19. The pharmaceutical composition of claim 16 and at least one further active ingredient.

20. The medicament as claimed in claim 17, which comprises, as a further active ingredient, one or more antidiabetics, active hypoglycemic ingredients, HMG-CoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbers, LDL receptor inducers, ACAT inhibitors, antioxidants, lipoprotein lipase inhibitors, ATP citrate lyase inhibitors, squalene synthetase inhibitors, lipoprotein(a) antagonists, HM74A receptor agonists, lipase inhibitors, insulins, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose 1,6-biphosphatase, modulators of glucose transporter 4, inhibitors of glutamine: fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, modulators of GPR40, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY antagonists, MC4 agonists, orexin antagonists, H3 antagonists, TNF agonists, CRF antagonists, CRF BP antagonists, urocortin agonists, β3 agonists, CB1 receptor antagonists, MSH (melanocyte-stimulating hormone) agonists, MCH antagonists, CCK agonists, serotonin reuptake inhibitors, mixed serotoninergic and noradrenergic compounds, 5HT modulators, bombesin agonists, galanin antagonists, growth hormones, growth hormone-releasing compounds, TRH agonists, decoupling protein 2 or 3 modulators, leptin agonists, DA agonists (bromocriptin, doprexin), lipase/amylase inhibitors, PPAR modulators, RXR modulators or TR-β-agonists or amphetamines.

21. The medicament as claimed in claim 18, which comprises, as a further active ingredient, one or more antidiabetics, active hypoglycemic ingredients, HMG-CoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbers, LDL receptor inducers, ACAT inhibitors, antioxidants, lipoprotein lipase inhibitors, ATP citrate lyase inhibitors, squalene synthetase inhibitors, lipoprotein(a) antagonists, HM74A receptor agonists, lipase inhibitors, insulins, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose 1,6-biphosphatase, modulators of glucose transporter 4, inhibitors of glutamine: fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, modulators of GPR40, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY antagonists, MC4 agonists, orexin antagonists, H3 antagonists, TNF agonists, CRF antagonists, CRF BP antagonists, urocortin agonists, β3 agonists, CB1 receptor antagonists, MSH (melanocyte-stimulating hormone) agonists, MCH antagonists, CCK agonists, serotonin reuptake inhibitors, mixed serotoninergic and noradrenergic compounds, 5HT modulators, bombesin agonists, galanin antagonists, growth hormones, growth hormone-releasing compounds, TRH agonists, decoupling protein 2 or 3 modulators, leptin agonists, DA agonists (bromocriptin, doprexin), lipase/amylase inhibitors, PPAR modulators, RXR modulators or TR-β-agonists or amphetamines.

22. The medicament as claimed in claim 19, which comprises, as a further active ingredient, one or more antidiabetics, active hypoglycemic ingredients, HMG-CoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbers, LDL receptor inducers, ACAT inhibitors, antioxidants, lipoprotein lipase inhibitors, ATP citrate lyase inhibitors, squalene synthetase inhibitors, lipoprotein(a) antagonists, HM74A receptor agonists, lipase inhibitors, insulins, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose 1,6-biphosphatase, modulators of glucose transporter 4, inhibitors of glutamine: fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, modulators of GPR40, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY antagonists, MC4 agonists, orexin antagonists, H3 antagonists, TNF agonists, CRF antagonists, CRF BP antagonists, urocortin agonists, β3 agonists, CB1 receptor antagonists, MSH (melanocyte-stimulating hormone) agonists, MCH antagonists, CCK agonists, serotonin reuptake inhibitors, mixed serotoninergic and noradrenergic compounds, 5HT modulators, bombesin agonists, galanin antagonists, growth hormones, growth hormone-releasing compounds, TRH agonists, decoupling protein 2 or 3 modulators, leptin agonists, DA agonists (bromocriptin, doprexin), lipase/amylase inhibitors, PPAR modulators, RXR modulators or TR-β-agonists or amphetamines.

23. A method of treating metabolic syndrome, diabetes, obesity, weight reduction, nicotine dependence, alcohol dependence, CNS disorders, schizophrenia and Alzheimer's comprising administering a pharmaceutically acceptable amount of the compound of claim 1 to a person in need thereof.

24. A method of treating metabolic syndrome, diabetes, obesity, weight reduction, nicotine dependence, alcohol dependence, CNS disorders, schizophrenia and Alzheimer's comprising administering a pharmaceutically acceptable amount of the compound of claim 5 to a person in need thereof.

25. A method of treating metabolic syndrome, diabetes, obesity, weight reduction, nicotine dependence, alcohol dependence, CNS disorders, schizophrenia and Alzheimer's comprising administering a pharmaceutically acceptable amount of the compound of claim 9 to a person in need thereof.

26. A process for producing a pharmaceutical composition comprising one or more of the compounds as claimed in claim 1, which comprises mixing the active ingredient with a pharmaceutically suitable carrier and bringing this mixture into a form suitable for administration.

27. A process for producing a pharmaceutical composition comprising one or more of the compounds as claimed in claim 1, which comprises mixing the active ingredient with a pharmaceutically suitable carrier and bringing this mixture into a form suitable for administration.

28. A process for producing a pharmaceutical composition comprising one or more of the compounds as claimed in claim 9, which comprises mixing the active ingredient with a pharmaceutically suitable carrier and bringing this mixture into a form suitable for administration.
